(19) United States
(12) Patent Application Publication
(10) Pub. No.: US 2024/0262830 A1
(43) Pub. Date: Aug. 8, 2024

(54) IRE1A MODULATORS AND USES THEREOF

(71) Applicant: Nimbus Iaso, Inc., Cambridge, MA (US)

(72) Inventors: Kevin KREUTTER, Arlington, MA (US); Lewis WHITEHEAD, Swampscott, MA (US); Donna L. ROMERO, Chesterfield, MO (US); Steven K. ALBANESE, Brooklyn, NY (US); Heidi KOLDSOE, New York, NY (US); Andreas VERRAS, New York, NY (US)

(21) Appl. No.: 17/797,379

(22) PCT Filed: Feb. 2, 2021
(86) PCT No.: PCT/US2021/016164

§ 371 Date: Aug. 3, 2022

Related U.S. Application Data
(60) Provisional application No. 62/969,435, filed on Feb. 3, 2020.

(51) Int. Cl. C07D 487/04; A61K 31/517; A61K 31/519; A61K 31/53; A61K 31/55; C07D 401/14; C07D 495/04

(52) U.S. Cl. CPC C07D 487/04; A61K 31/517; A61K 31/519; A61K 31/53; A61K 31/55; C07D 401/14; C07D 495/04

(57) ABSTRACT

The present invention provides compounds, compositions thereof, and methods of using the same for the modulation of IRE1α, and the treatment of IRE1α-mediated disorders.

### CROSS-REFERENCE TO RELATED APPLICATIONS

The present application claims priority to U.S. provisional patent application Ser. No. 62/969,435, filed Feb. 3, 2020, the entirety of which is incorporated herein by reference.

### TECHNICAL FIELD OF THE INVENTION

The present invention relates to compounds and methods useful for modulating inositol-requiring enzyme 1α (IRE1α). The invention also provides pharmaceutically acceptable compositions comprising compounds of the present invention and methods of using said compositions in the treatment of various disorders.

### BACKGROUND OF THE INVENTION

The kinase/endoribonuclease inositol requiring enzyme 1 (IRE1α) is a key player in the unfolded protein response (UPR) that is involved in regulating cellular responses to stress. IRE1α resides in the endoplasmic reticulum. It is a protein containing a luminal domain that binds to misfolded proteins, a transmembrane segment, and a cytoplasmic-facing portion consisting of a kinase moiety and a tandem endoribonuclease domain. IRE1α possesses intrinsic kinase activity and an endoribonuclease activity. Upon activation, IRE1α oligomerizes and carries out an unconventional RNA splicing activity, removing an intron from the X-box binding protein 1 (XBP1) mRNA, to allow it to become translated into a functional transcription factor, XBP1s. XBP1s upregulates ER chaperones and endoplasmic reticulum associated degradation (ERAD) genes that facilitate recovery from ER stress. IRE1α also mediates Regulated IRE1-dependent Decay of mRNA (RIDD), where IRE1α RNAse activity selectively degrades mRNAs and miRNAs to maintain cellular homeostasis or regulate cell fate (reviews Coelho & Domingos, Frontiers Genetics 2014; Maurel et al. Trends Biochem Sci 2014). Independent of its RNAse function, IRE1α has also been reported to modulate cell responses via association with signaling proteins (review Coelho & Domingos, Frontiers Genetics 2014).

While IRE1α and the UPR mediate cytoprotective and pro-survival functions of the UPR in response to acute cell stress, they can also lead to apoptosis and aberrant outcomes under conditions of chronic or irremediable stress that contribute to disease pathology. IRE1α activity has been implicated in autoimmune, neurodegenerative, fibrotic, metabolic disease, as well as in some tumor cells, and thus is considered a potential therapeutic target. For example, high levels of spliced XBP1 and protection from apoptosis have been observed in multiple myeloma (Harnoss et al. PNAS 2019) and breast tumor cells (Chen et al. Nature 2014; McGrath et al. Cancers 2018); hyperactivation of IRE1 in diabetes may contribute to apoptosis of 3 islet cells in diabetes (Ghosh et al. Molec Metab 2019) and upregulated ER stress and XBP1 target genes have been reported in fribrosis (Kim et al. Sci Rep 2016; Chen et al., Am J Respir Crit Care Med 2019; Kropski and Blackwell J Clin Invest 2018). Conversely, knockdown of IRE1 or XBP1 leads to reduced tumor growth, improved insulin secretion and attenuated fibrosis (Zhao et al. J Clin Invest 2018; Ghosh et al. Molec Metab 2019; Heindryckx et al. EMBO Molec Med 2016).

IRE1α activity is upregulated by ER stress that induces the oligomerization and subsequent transphosphorylation of IRE1α. The phosphorylation of specific activation loop residues in the IRE1 kinase domain increases IRE1-mediated XBP1 splicing and IRE1 RNAse activity (Prischi et al. Nature Comm 2014). Thus, inhibitors have been described that bind the ribonuclease domain or the kinase domain, that block the function of IRE1α (Concha et al. Molec Pharmacol 2015; Harrington et al. ACS Med Chem Lett 2015).

### SUMMARY OF THE INVENTION

It has now been found that compounds of this invention, and pharmaceutically acceptable compositions thereof, are effective as modulators of IRE1α. In certain embodiments, the invention provides for antagonists of IRE1α. In certain embodiments, the invention provides for agonists of IRE1α. In certain embodiments, the invention provides for compounds of the formulae presented herein.

Compounds of the present invention, and pharmaceutically acceptable compositions thereof, are useful for treating a variety of diseases, disorders or conditions, associated with regulation of signaling pathways implicating IRE1α. Such diseases, disorders, or conditions include those described herein.

### DETAILED DESCRIPTION OF CERTAIN EMBODIMENTS

### 1. General Description of Certain Embodiments of the Invention

In certain aspects, the present invention provides a compound of formula I′:

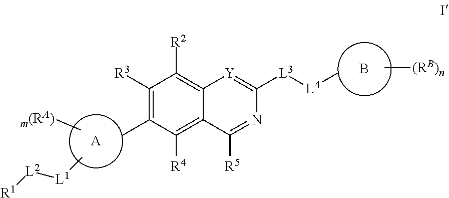

or a pharmaceutically acceptable salt thereof, wherein each of Ring A, Ring B, Y, L¹, L², L³, L⁴, R¹, R², R³, R⁴, R⁵, R^{A}, R^{B}, m, and n, is as defined below and described in embodiments herein, both singly and in combination.

In some embodiments, the present invention provides a pharmaceutical composition comprising a compound of formula I′, and a pharmaceutically acceptable carrier, adjuvant, or diluent.

In some embodiments, the present invention provides a method of treating a IRE1α-mediated disease, disorder, or condition comprising administering to a patient in need thereof, a a compound of formula I′ or a pharmaceutically acceptable salt thereof.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

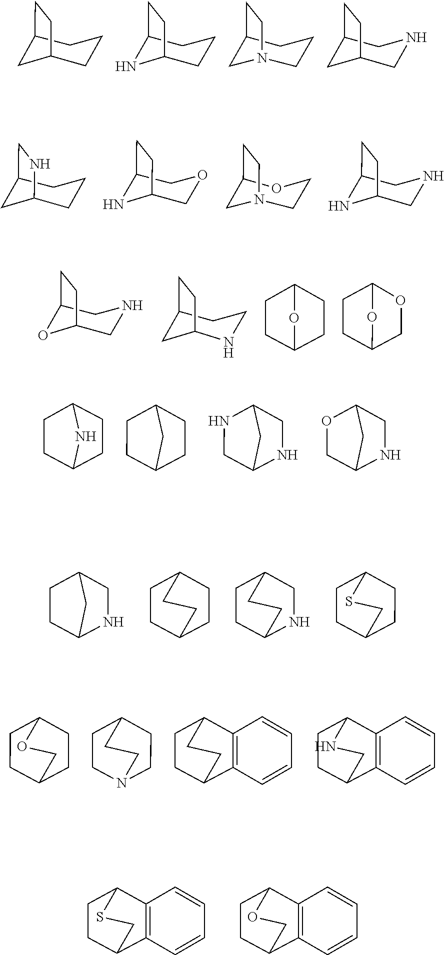

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —N(R^{∘})C(NR^{∘})N(R^{∘})₂; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms. In certain embodiments, Ring B of a provided compound may be substituted with one or more deuterium atoms.

In certain embodiments, the compounds of the invention include all N-oxide forms of the compounds.

As used herein, a “IRE1α antagonist” or a “IRE1α inhibitor” is a molecule that reduces, inhibits, or otherwise diminishes one or more of the biological activities of IRE1α. Antagonism using the IRE1α antagonist does not necessarily indicate a total elimination of the IRE1α activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of IRE1α compared to an appropriate control.

By “specific antagonist” is intended an agent that reduces, inhibits, or otherwise diminishes the activity of a defined target greater than that of an unrelated target. For example, a IRE1α specific antagonist reduces at least one biological activity of IRE1α by an amount that is statistically greater than the inhibitory effect of the antagonist on any other protein. In some embodiments, the IC₅₀ of the antagonist for the target is about 90%, 80%, 70%, 60%, 50%, 40%, 30%, 20%, 10%, 5%, 1%, 0.1%, 0.01%, 0.001% or less of the IC₅₀ of the antagonist for a non-target. The presently disclosed compounds may or may not be a specific IRE1α antagonist. A specific IRE1α antagonist reduces the biological activity of IRE1α by an amount that is statistically greater than the inhibitory effect of the antagonist on any other protein. In some of these embodiments, the IC₅₀ of the IRE1α antagonist for IRE1α is about 90%, 80%, 70%, 60%, 50%, 40%, 30%, 20%, 10%, 0.1%, 0.01%, or 0.001%.

As used herein, a “IRE1α agonist“or a” IRE1α enhancer” is a molecule that enhances, or increases one or more of the biological activities of IRE1α. The activity could increase by a statistically significant amount including, for example, an increase of at least about 5%, 10%, 15%, 20%, 25%, 30%, 35%, 40%, 45%, 50%, 55%, 60%, 65%, 70%, 75%, 80%, 85%, 95% or 100% of the activity of IRE1α compared to an appropriate control.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in IRE1α activity between a sample comprising a compound of the present invention, or composition thereof, and IRE1α, and an equivalent sample comprising IRE1α, in the absence of said compound, or composition thereof.

### 3. Description of Exemplary Embodiments

In one aspect, the present invention provides a compound of formula I′:

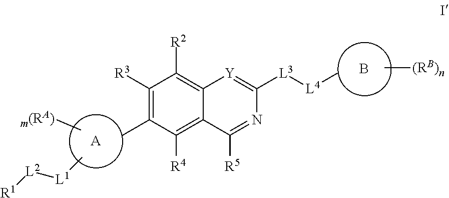

or a pharmaceutically acceptable salt thereof, wherein:
Ring A is a 7-12-membered fused bicyclic saturated, partially unsaturated, or unsaturated, carbocyclic, aryl, heterocyclic, or heteroaryl ring system, having 0-5 heteroatoms independently selected from nitrogen, oxygen, and sulfur;
L¹ is —O—, —S—, —NR—, —S(O)₂—, —S(O)₂NR—, —S(O)—, —S(O)NR—, —C(O)—, —C(O)O—, —C(O)NR—, —N(R)C(O)N(R)—, —C(O)N(R)O—, —OC(O)—, —OC(O)NR—, —N(R)C(O)O—, —N(R)C(O)—, —N(R)S(O)₂—; or L¹ is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
L² is a covalent bond or L² is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
R¹ is selected from C₁₋₆ aliphatic; phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; and a 3-7 membered saturated or partially unsaturated monocyclic heterocyclic ring having 1-2 heteroatoms independently selected from nitrogen, oxygen, and sulfur; each of which is substituted with q instances of R^{C};
each instance of R^{A} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{A} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D};
Y is N or CR;
each of R², R³, R⁴, and R⁵, is independently selected from hydrogen, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, halogen, an optionally substituted C₁₋₆ aliphatic; optionally substituted phenyl; optionally substituted naphthalenyl; an optionally substituted 3-7 membered saturated or partially unsaturated monocyclic carbocyclic ring; an optionally substituted 3-7 membered saturated or partially unsaturated monocyclic heterocyclic ring having 1-2 heteroatoms independently selected from nitrogen, oxygen, phosphorous, silicon and sulfur; and an optionally substituted 5-6 membered monocyclic heteroaryl ring having 1-4 heteroatoms independently selected from nitrogen, oxygen, and sulfur; each of which is substituted with r instances of R and s instances of R^{D};
Ring B is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 6-10 membered saturated or partially unsaturated spirocyclic, fused, or bridged ring having 0-3 heteroatoms independently selected from nitrogen, oxygen, and sulfur; each of which is substituted with q instances of R^{C};
L³ is —O—, —S—, —NR—, —S(O)₂—, —S(O)₂NR—, —S(O)—, —S(O)NR—, —C(O)—, —C(O)O—, —C(O)NR—, —C(O)N(R)O—, —OC(O)—, —OC(O)NR—, —N(R)C(O)O—, —N(R)C(O)N(R)—, —N(R)C(O)—, —N(R)S(O)₂—; or L³ is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
L⁴ is a covalent bond or L⁴ is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
each instance of R^{B} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{B} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D};
each instance of R^{C} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{C} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D};
each instance of R^{D} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R²;
each R is independently hydrogen, —CN, halogen, or an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur;
m is 0, 1, 2, 3, or 4;
n is 0, 1, 2, 3, or 4;
each q is independently 0, 1, 2, 3, or 4;
each r is independently 0, 1, 2, 3, or 4; and
each s is independently 0, 1, 2, 3, or 4.

In one aspect, the present invention provides a compound of formula I:

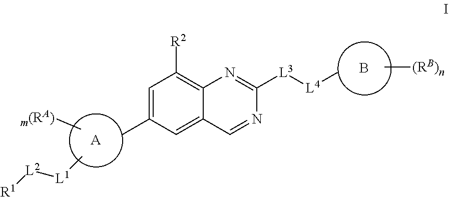

or a pharmaceutically acceptable salt thereof, wherein:
Ring A is a 7-12-membered fused bicyclic saturated, partially unsaturated, or unsaturated, carbocyclic, aryl, heterocyclic, or heteroaryl ring system, having 0-5 heteroatoms independently selected from nitrogen, oxygen, and sulfur;
L¹ is —O—, —S—, —NR—, —S(O)₂—, —S(O)₂NR—, —S(O)—, —S(O)NR—, —C(O)—, —C(O)O—, —C(O)NR—, —N(R)C(O)N(R)—, —C(O)N(R)O—, —OC(O)—, —OC(O)NR—, —N(R)C(O)O—, —N(R)C(O)—, —N(R)S(O)₂—; or L¹ is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
L² is a covalent bond or L² is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
R¹ is selected from C₁₋₆ aliphatic; phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; and a 3-7 membered saturated or partially unsaturated monocyclic heterocyclic ring having 1-2 heteroatoms independently selected from nitrogen, oxygen, and sulfur; each of which is substituted with q instances of R^{C};
each instance of R^{A} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{A} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D};
R² is H, C₁₋₆ aliphatic, or an optionally substituted 3-7 membered saturated or partially unsaturated monocyclic carbocyclic ring;
Ring B is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 6-10 membered saturated or partially unsaturated spirocyclic, fused, or bridged ring having 0-3 heteroatoms independently selected from nitrogen, oxygen, and sulfur; each of which is substituted with q instances of R^{C};
L³ is —O—, —S—, —NR—, —S(O)₂—, —S(O)₂NR—, —S(O)—, —S(O)NR—, —C(O)—, —C(O)O—, —C(O)NR—, —C(O)N(R)O—, —OC(O)—, —OC(O)NR—, —N(R)C(O)O—, —N(R)C(O)N(R)—, —N(R)C(O)—, —N(R)S(O)₂—; or L³ is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
L⁴ is a covalent bond or L⁴ is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
each instance of R^{B} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{B} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D};
each instance of R^{C} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{C} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D};
each instance of R^{D} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R²;
each R is independently hydrogen, —CN, halogen, or an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur;
m is 0, 1, 2, 3, or 4;
n is 0, 1, 2, 3, or 4;
each q is independently 0, 1, 2, 3, or 4;
each r is independently 0, 1, 2, 3, or 4; and
each s is independently 0, 1, 2, 3, or 4.

In some embodiments, Ring A is a 8-10-membered fused bicyclic saturated, partially unsaturated, or unsaturated, heterocyclic, or heteroaryl ring system, having 1-3 heteroatoms independently selected from nitrogen, oxygen, and sulfur.

In some embodiments, Ring A is a 9-membered fused bicyclic saturated, partially unsaturated, or unsaturated, heterocyclic, or heteroaryl ring system, having 1-3 heteroatoms independently selected from nitrogen, oxygen, and sulfur.

In some embodiments, Ring A is

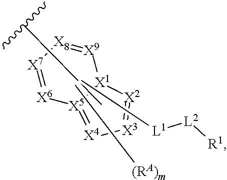

wherein each of X¹, X², X³, X⁴, X⁵, X⁶, X⁷, X⁸, and X⁹, is independently C, CR, N, or S, as allowed by valance.

In some embodiments, Ring A

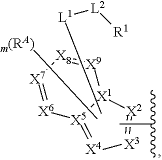

wherein each of X¹, X², X³, X⁴, X⁵, X⁶, X⁷, X⁸, and X⁹, is independently C, CR, N, or S, as allowed by valance.

In some embodiments, Ring A is

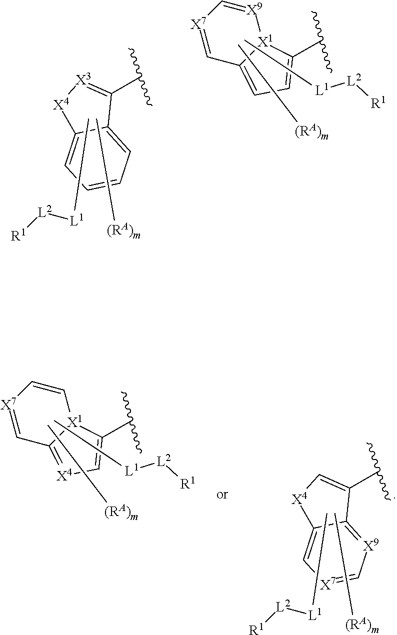

In some embodiments, Ring A is

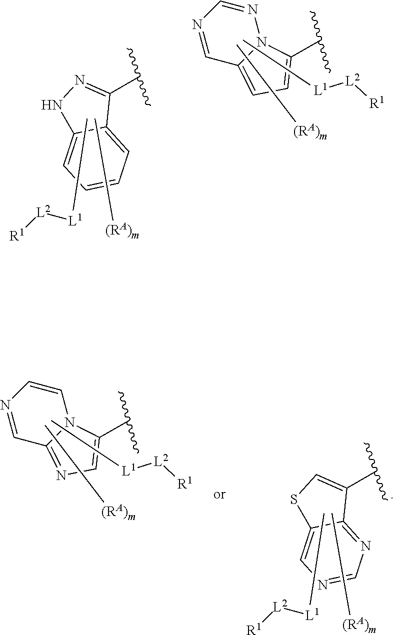

In some embodiments, Ring A is selected from those depicted in Table 1, below.

In some embodiments, L¹ is —S—, —NR—, —S(O)₂—, —S(O)₂NR—, —S(O)—, —S(O)NR—, —C(O)—, —C(O)O—, —C(O)NR—, —C(O)N(R)O—, —N(R)C(O)N(R)—, —N(R)C(O)O—, —N(R)C(O)—, —N(R)S(O)₂—; or L¹ is a C₁₋₄ bivalent saturated or unsaturated, straight or branched hydrocarbon chain wherein one or two methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—.

In some embodiments, L¹ is —S—, —NR—, —S(O)₂—, —S(O)₂NR—, —S(O)—, —S(O)NR—, —C(O)—, —C(O)O—, —C(O)NR—, —C(O)N(R)O—, —N(R)C(O)N(R)—, —N(R)C(O)O—, —N(R)C(O)—, or —N(R)S(O)₂—.

In some embodiments, L¹ is —NR—, —N(R)C(O)N(R)—, or —N(R)S(O)₂—.

In some embodiments, L¹ is selected from those depicted in Table 1, below.

In some embodiments, L² is a covalent bond or L² is a C₁₋₄ bivalent saturated or unsaturated, straight or branched hydrocarbon chain wherein one or two methylene units of the chain are optionally and independently replaced by —C(R)₂—, —N(R)—, —N(R)C(O)—, —C(O)N(R)—, or —N(R)S(O)₂—.

In some embodiments, L² is a covalent bond or L² is a C₁₋₄ bivalent saturated or unsaturated, straight or branched hydrocarbon chain.

In some embodiments, L² is a covalent bond or L² is a C₁₋₄ bivalent saturated straight hydrocarbon chain wherein one or two methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—.

In some embodiments, L² is selected from those depicted in Table 1, below.

In some embodiments, R¹ is selected from C₂₋₆ aliphatic; phenyl; a 3-6 membered saturated or partially unsaturated monocyclic carbocyclic ring; a 5-6 membered monocyclic heteroaryl ring having 1-4 heteroatoms independently selected from nitrogen, oxygen, and sulfur; and a 5-7 membered saturated or partially unsaturated monocyclic heterocyclic ring having 1-2 heteroatoms independently selected from nitrogen, oxygen, and sulfur; each of which is substituted with q instances of R^{C}.

In some embodiments, R¹ is selected from ethyl, propyl, i-Pr, n-Bu, s-Bu, t-Bu, straight chain or branched pentyl, straight chain or branched hexyl, cyclopropyl, cyclobutyl, cyclopentyl, cyclohexyl, cycloheptyl, phenyl, furanyl, furazanyl, imidazolidinyl, imidazolinyl, imidazolyl, isothiazolyl, isoxazolyl, morpholinyl, oxadiazolyl, 1,2,3-oxadiazolyl, 1,2,4-oxadiazolyl; 1,2,5-oxadiazolyl, 1,3,4-oxadiazolyl, oxazolidinyl, oxazolyl, oxazolidinyl, oxetanyl, pyrimidinyl, piperazinyl, piperidinyl, pyranyl, pyrazinyl, pyrazolidinyl, pyrazolinyl, pyrazolyl, pyridazinyl, pyridinyl, pyridyl, pyrimidinyl, pyrrolidinyl, pyrrolinyl, 2H-pyrrolyl, pyrrolyl, tetrahydrofuranyl, tetrahydropyranyl, thiazolyl, thienyl, triazinyl, 1,2,3-triazolyl, 1,2,4-triazolyl, 1,2,5-triazolyl, 1,3,4-triazolyl, oxetanyl, azetidinyl, or xanthenyl; each of which is substituted by q instances of R^{C}.

In some embodiments, R¹ is selected from ethyl, propyl, i-Pr, n-Bu, cyclopropyl, cyclobutyl, cyclopentyl, cyclohexyl, cycloheptyl, phenyl, furanyl, imidazolidinyl, imidazolinyl, imidazolyl, isothiazolyl, isoxazolyl, morpholinyl, oxadiazolyl, oxazolidinyl, oxazolyl, oxazolidinyl, oxetanyl, pyrimidinyl, piperazinyl, piperidinyl, pyranyl, pyrazinyl, pyrazolidinyl, pyrazolinyl, pyrazolyl, pyridazinyl, pyridinyl, pyridyl, pyrimidinyl, pyrrolidinyl, pyrrolinyl, 2H-pyrrolyl, pyrrolyl, thiazolyl, thienyl, triazinyl, oxetanyl, azetidinyl, or xanthenyl; each of which is substituted by q instances of R^{C}.

In some embodiments, R¹ is selected from ethyl, propyl, i-Pr, n-Bu, cyclopropyl, cyclobutyl, cyclopentyl, cyclohexyl, cycloheptyl, phenyl, pyrimidinyl, piperazinyl, piperidinyl, pyranyl, pyrazinyl, pyrazolidinyl, pyrazolinyl, pyrazolyl, pyridazinyl, pyridinyl, pyridyl, pyrimidinyl, pyrrolidinyl, pyrrolinyl, 2H-pyrrolyl, or pyrrolyl; each of which is substituted by q instances of R^{C}.

In some embodiments, R¹ is selected from -Et, —Pr, —CH₂CF₃, —CF₃, —OCF₃, —CHF₂, —CF₂CH₃, —CF₂CF₃, —SF5, CN,

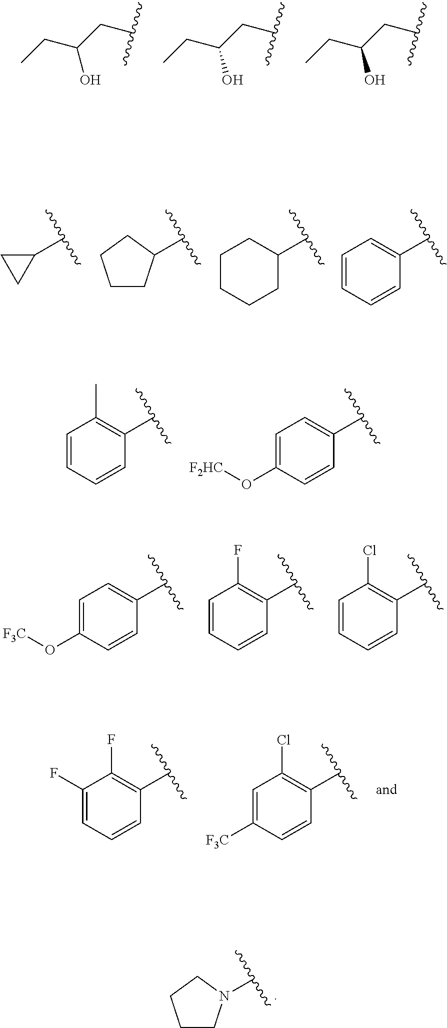

In some embodiments, R¹ is selected from those depicted in Table 1, below.

In certain embodiments, each R^{A} is independently halogen, —CN, —NO₂, —OR, —NR₂, —S(O)₂R, —S(O)₂NR₂, —S(O)R, —S(O)NR₂, —C(O)R, —C(O)OR, —C(O)NR₂, —OC(O)R, —OC(O)NR₂, —N(R)C(O)R, —N(R)S(O)₂R, or —N(R)S(O)R; or each instance of R^{A} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D}.

In certain embodiments, each R^{A} is independently halogen, —CN, —NO₂, —OR, —NR₂, —S(O)₂R, —S(O)₂NR₂, —S(O)R, —S(O)NR₂, —C(O)R, —C(O)OR, —C(O)NR₂, —OC(O)R, —OC(O)NR₂, —N(R)C(O)R, —N(R)S(O)₂R, or —N(R)S(O)R; or each instance of R^{A} is independently an optionally substituted group selected from C₁₋₆ aliphatic; phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; each of which is substituted with r instances of R and s instances of R^{D}.

In certain embodiments, each R^{A} is independently halogen, —OR, —NR₂, —C(O)R, —C(O)OR, —C(O)NR₂, —OC(O)R, —OC(O)NR₂, —N(R)C(O)R, —N(R)S(O)₂R, or —N(R)S(O)R; or each instance of R^{A} is independently an optionally substituted group selected from C₁₋₆ aliphatic; each of which is substituted with r instances of R and s instances of R^{D}.

In certain embodiments, each R^{A} is independently —F, —Cl, —Br, —I, methyl, ethyl, propyl, i-propyl, n-butyl, s-butyl, t-butyl, straight chain or branched pentyl, or straight chain or branched hexyl; each of which is substituted with r instances of R and s instances of R^{D}.

In some embodiments, each R^{A} is independently selected from -Et, —Pr, —CH₂CF₃, —CF₃, —OCF₃, —CHF₂, —CF₂CH₃, —CF₂CF₃, —SF5, and CN.

In certain embodiments, each R^{A} is independently —F.

In some embodiments, each R^{A} is independently selected from those depicted in Table 1, below.

In certain embodiments, Ring A, L¹, L², R¹, and R^{A}, together, are selected from

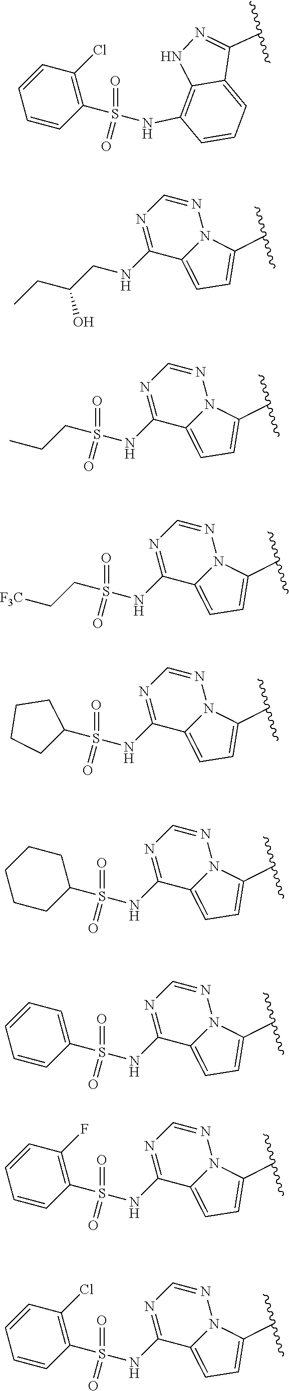

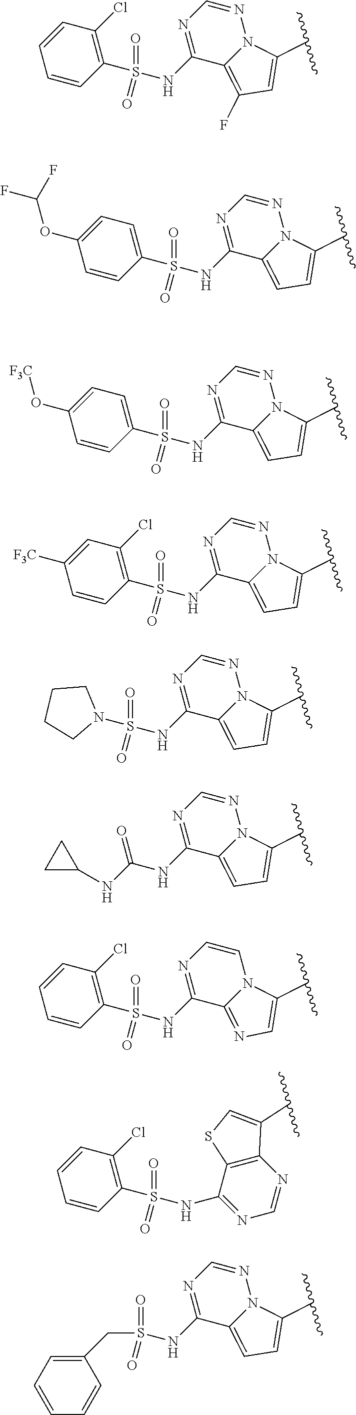

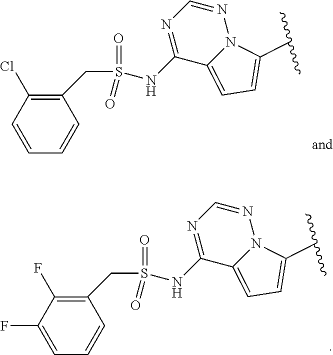 [SMILES: *c1ccc2c(NS(=O)(=O)Cc3ccccc3Cl)ncnn12 ; *c1ccc2c(NS(=O)(=O)Cc3cccc(F)c3F)ncnn12]

In certain embodiments, R² is H or C₁₋₆ aliphatic.

In certain embodiments, R² is H, methyl, ethyl, propyl, i-propyl, n-butyl, s-butyl, t-butyl, straight chain or branched pentyl, or straight chain or branched hexyl; or R² is —CH₂CF₃, —CF₃, —OCF₃, —CHF₂, —CF₂CH₃, —CF₂CF₃, —SF5, or CN.

In certain embodiments, R² is H.

In certain embodiments, R² is methyl or ethyl. In certain embodiments, R² is ethyl.

In certain embodiments, R² is an optionally substituted 3-7 membered saturated or partially unsaturated monocyclic carbocyclic ring. In certain embodiments, R² is cyclopropyl, cyclobutyl, cyclopentyl, cyclohexyl, or cyclopentyl; each of which is optionally substituted.

In some embodiments, R² is selected from those depicted in Table 1, below.

In certain embodiments, each of R³, R⁴, and R⁵, is independently selected from H, methyl, ethyl, propyl, i-propyl, n-butyl, s-butyl, t-butyl, straight chain or branched pentyl, and straight chain or branched hexyl; or each of R³, R⁴, and R⁵, is independently selected from —CH₂CF₃, —CF₃, —OCF₃, —CHF₂, —CF₂CH₃, —CF₂CF₃, —SF5, and CN.

In some embodiments, L³ is —O—, —NR—, —S(O)₂—, —S(O)₂NR—, —S(O)—, —S(O)NR—, —C(O)—, —C(O)O—, —C(O)NR—, —C(O)N(R)O—, —OC(O)—, —OC(O)NR—, —N(R)C(O)O—, —N(R)C(O)—, —N(R)S(O)₂—; or L³ is a C₁₋₄ bivalent saturated or unsaturated, straight or branched hydrocarbon chain wherein one or two methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, or —C(O)O—.

In some embodiments, L³ is —O—, —NR—, —C(O)—, —C(O)O—, —C(O)NR—, —C(O)N(R)O—, —OC(O)—, —OC(O)NR—, —N(R)C(O)O—, —N(R)C(O)—, —N(R)S(O)₂—; or L³ is a C₁₋₄ bivalent saturated or unsaturated, straight or branched hydrocarbon chain.

In some embodiments, L³ is —NR—, —C(O)—, —C(O)O—, —C(O)NR—, —N(R)C(O)—, —N(R)S(O)₂—; or L³ is a C₁₋₄ bivalent saturated or unsaturated, straight or branched hydrocarbon chain.

In some embodiments, L³ is —NR—. In some embodiments, L³ is —NH—.

In some embodiments, L³ is selected from those depicted in Table 1, below.

In some embodiments, L⁴ is a covalent bond or L⁴ is a C₁₋₂ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—.

In some embodiments, L⁴ is a covalent bond or L⁴ is C₁ bivalent saturated hydrocarbon chain.

In some embodiments, L⁴ is a covalent bond. In some embodiments, L⁴ is —C(R)₂—. In some embodiments, L⁴ is —CH₂—.

In some embodiments, L⁴ is selected from those depicted in Table 1, below.

In some embodiments, Ring B is selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur; each of which is substituted with q instances of R^{C}.

In some embodiments, Ring B is selected from a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; and a 6-10 membered saturated or partially unsaturated spirocyclic ring having 0-3 heteroatoms independently selected from nitrogen, oxygen, and sulfur.

In some embodiments, Ring B is selected from

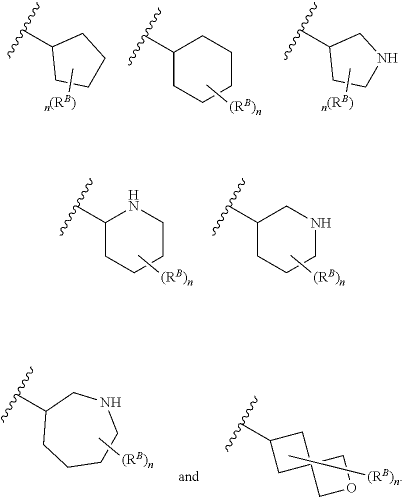

In some embodiments, Ring B is selected from those depicted in Table 1, below.

In certain embodiments, each instance of R^{B} is independently halogen, —CN, —OR, —NR₂, —C(O)R, —C(O)OR, —C(O)NR₂, —OC(O)R, —OC(O)NR₂, —N(R)C(O)OR, —N(R)C(O)R, —N(R)C(O)NR₂, —N(R)S(O)₂R, —N(R)S(O)R; or each instance of R^{B} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; each of which is substituted with r instances of R and s instances of R^{D}.

In certain embodiments, each instance of R^{B} is independently halogen, —CN, —OR, —NR₂, —C(O)R, —C(O)OR, —C(O)NR₂, —OC(O)R, —OC(O)NR₂, —N(R)C(O)OR, —N(R)C(O)R, —N(R)C(O)NR₂, —N(R)S(O)₂R, —N(R)S(O)R; or each instance of R^{B} is independently an optionally substituted C₁₋₆ aliphatic; which is substituted with r instances of R and s instances of R^{D}.

In certain embodiments, each instance of R^{B} is independently —OR, or —NR₂.

In certain embodiments, L³, L⁴, Ring B, and R^{B} is

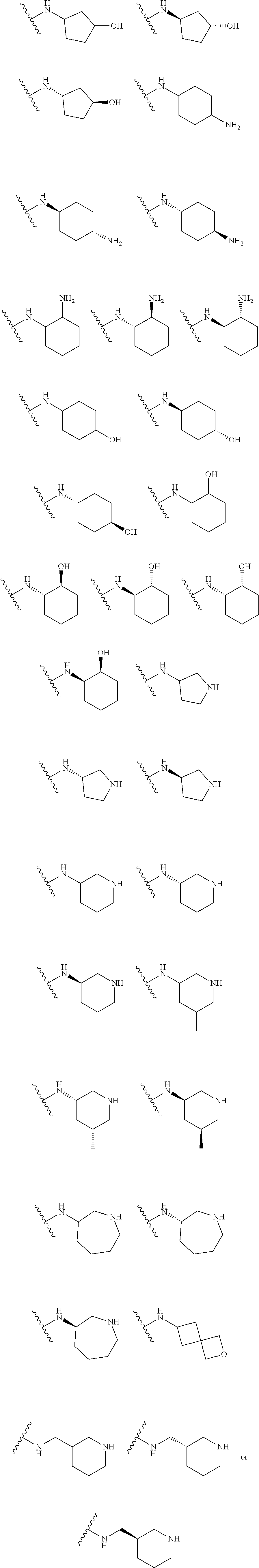

In certain embodiments, each instance of R^{B} is independently selected from —CH₂CF₃, —CF₃, —OCF₃, —CHF₂, —CF₂CH₃, —CF₂CF₃, —SF5, and CN.

In some embodiments, each instance of R^{C} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{C} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur.

In some embodiments, each instance of R^{C} is oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂.

In some embodiments, each instance of R^{C} is an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur.

In some embodiments, each instance of R^{C} is selected from those depicted in Table 1, below.

As defined generally above, each R is independently hydrogen, or an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur.

In some embodiments, R is selected from those depicted in Table 1, below.

As defined generally above, each hydrogen bound to carbon can be optionally and independently replaced by deuterium.

In some embodiments, a hydrogen bound to carbon is replaced by deuterium.

As defined generally above, m is 0, 1, 2, 3, or 4. In some embodiments, m is 0. In some embodiments, m is 1, 2, 3, or 4. In some embodiments, m is 1. In some embodiments, m is 2. In some embodiments, m is 3. In some embodiments, m is 4. In some embodiments, m is selected from those depicted in Table 1, below.

As defined generally above, n is 0, 1, 2, 3, or 4. In some embodiments, n is 0. In some embodiments, n is 1, 2, 3, or 4. In some embodiments, n is 1. In some embodiments, n is 2. In some embodiments, n is 3. In some embodiments, n is 4. In some embodiments, n is selected from those depicted in Table 1, below.

As defined generally above, each q is independently 0, 1, 2, 3, or 4. In some embodiments, each q is independently 0. In some embodiments, each q is independently 1, 2, 3, or 4. In some embodiments, each q is independently 1. In some embodiments, each q is independently 2. In some embodiments, each q is independently 3. In some embodiments, each q is independently 4. In some embodiments, each q is independently selected from those depicted in Table 1, below.

As defined generally above, each r is independently 0, 1, 2, 3, or 4. In some embodiments, each r is independently 0. In some embodiments, each r is independently 1, 2, 3, or 4. In some embodiments, each r is independently 1. In some embodiments, each r is independently 2. In some embodiments, each r is independently 3. In some embodiments, each r is independently 4. In some embodiments, each r is independently selected from those depicted in Table 1, below.

As defined generally above, each s is independently 0, 1, 2, 3, or 4. In some embodiments, each s is independently 0. In some embodiments, each s is independently 1, 2, 3, or 4. In some embodiments, each s is independently 1. In some embodiments, each s is independently 2. In some embodiments, each s is independently 3. In some embodiments, each s is independently 4. In some embodiments, s is selected from those depicted in Table 1, below.

In some embodiments, the present invention provides a compound of formula I-a:

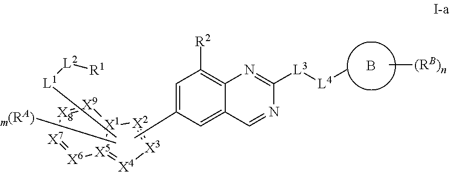

or a pharmaceutically acceptable salt thereof, wherein each of Ring B, L¹, L², L³, L⁴, R¹, R², R^{A}, R^{B}, X¹, X², X³, X⁴, X⁵, X⁶, X⁷, X⁸, X⁹, m, and n, is as defined above and described in embodiments herein, both singly and in combination.

In some embodiments, the present invention provides a compound of formula II:

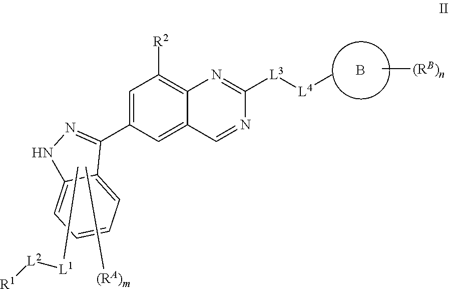

or a pharmaceutically acceptable salt thereof, wherein each of Ring B, L¹, L², L³, L⁴, R¹, R², R^{A}, R^{B}, m, and n, is as defined above and described in embodiments herein, both singly and in combination.

In some embodiments, the present invention provides a compound of formula III:

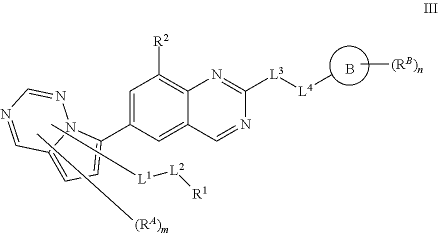

or a pharmaceutically acceptable salt thereof, wherein each of Ring B, L¹, L², L³, L⁴, R¹, R², R^{A}, R^{B}, m, and n, is as defined above and described in embodiments herein, both singly and in combination.

In some embodiments, the present invention provides a compound of formula IV:

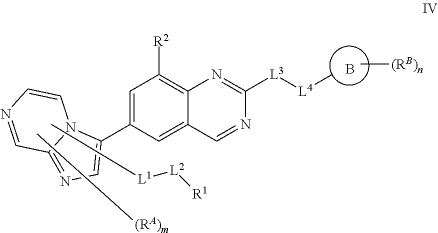

or a pharmaceutically acceptable salt thereof, wherein each of Ring B, L¹, L², L³, L⁴, R¹, R², R^{A}, R^{B}, m, and n, is as defined above and described in embodiments herein, both singly and in combination.

In some embodiments, the present invention provides a compound of formula IV-a:

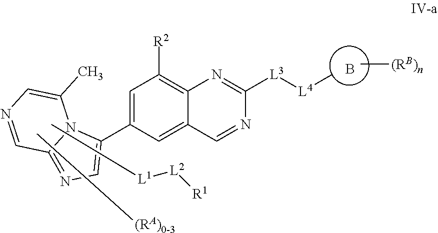

or a pharmaceutically acceptable salt thereof, wherein each of Ring B, L¹, L², L³, L⁴, R¹, R², R^{A}, R^{B}, and n, is as defined above and described in embodiments herein, both singly and in combination.

In some embodiments, the present invention provides a compound of formula V:

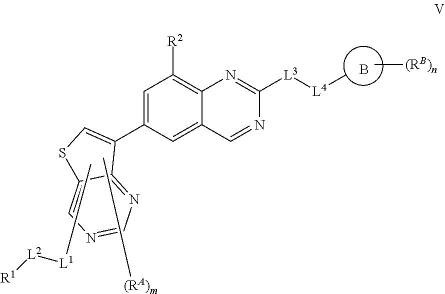

or a pharmaceutically acceptable salt thereof, wherein each of Ring B, L¹, L², L³, L⁴, R¹, R², R^{A}, R^{B}, m, and n, is as defined above and described in embodiments herein, both singly and in combination.

In some embodiments, the present invention provides a compound of formula VI:

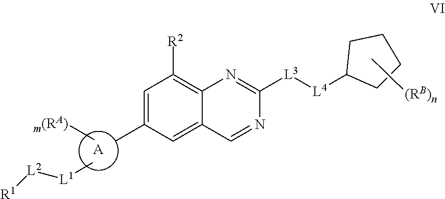

or a pharmaceutically acceptable salt thereof, wherein each of Ring A, L¹, L², L³, L⁴, R¹, R², R^{A}, R^{B}, m, and n, is as defined above and described in embodiments herein, both singly and in combination.

In some embodiments, the present invention provides a compound of formula VII:

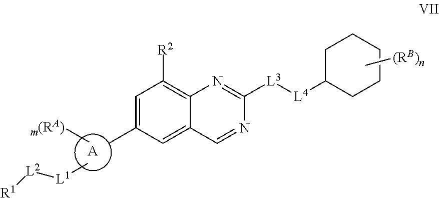

or a pharmaceutically acceptable salt thereof, wherein each of Ring A, L¹, L², L³, L⁴, R¹, R², R^{A}, R^{B}, m, and n, is as defined above and described in embodiments herein, both singly and in combination.

In some embodiments, the present invention provides a compound of formula VIII:

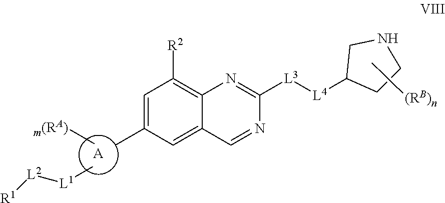

or a pharmaceutically acceptable salt thereof, wherein each of Ring A, L¹, L², L³, L⁴, R¹, R², R^{A}, R^{B}, m, and n, is as defined above and described in embodiments herein, both singly and in combination.

In some embodiments, the present invention provides a compound of formula IX:

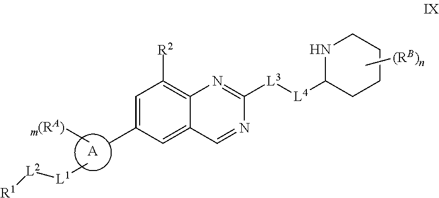

or a pharmaceutically acceptable salt thereof, wherein each of Ring A, L¹, L², L³, L⁴, R¹, R², R^{A}, R^{B}, m, and n, is as defined above and described in embodiments herein, both singly and in combination.

In some embodiments, the present invention provides a compound of formula X:

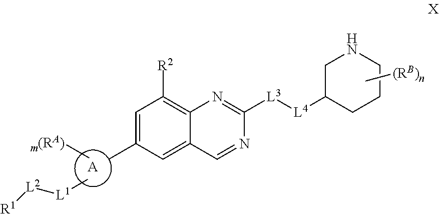

or a pharmaceutically acceptable salt thereof, wherein each of Ring A, L¹, L², L³, L⁴, R¹, R², R^{A}, R^{B}, m, and n, is as defined above and described in embodiments herein, both singly and in combination.

In some embodiments, the present invention provides a compound of formula XI:

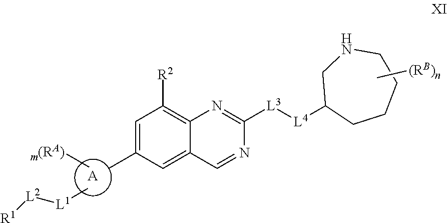

or a pharmaceutically acceptable salt thereof, wherein each of Ring A, L¹, L², L³, L⁴, R¹, R², R^{A}, R^{B}, m, and n, is as defined above and described in embodiments herein, both singly and in combination.

In some embodiments, the present invention provides a compound of formula XII:

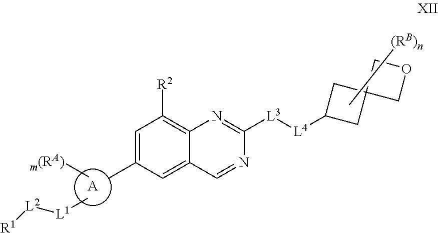

or a pharmaceutically acceptable salt thereof, wherein each of Ring A, L¹, L², L³, L⁴, R¹, R², R^{A}, R^{B}, m, and n, is as defined above and described in embodiments herein, both singly and in combination.

Exemplary compounds of the invention are set forth in Table 1, below.

TABLE 1
Selected Compounds

| Compound | Structure |
|---|---|
| I-1 | 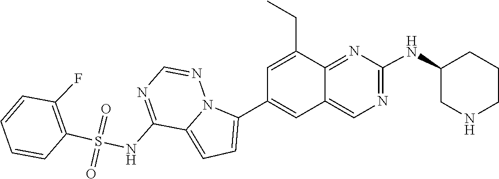 [SMILES: CCc1cc(-c2ccc3c(NS(=O)(=O)c4ccccc4F)ncnn23)cc2cnc(N[C@H]3CCCNC3)nc12] |
| I-2 | 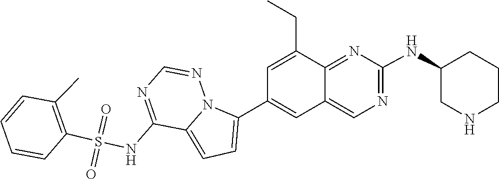 [SMILES: CCc1cc(-c2ccc3c(NS(=O)(=O)c4ccccc4C)ncnn23)cc2cnc(N[C@H]3CCCNC3)nc12] |
| I-3 | 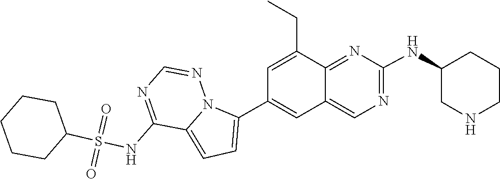 [SMILES: CCc1cc(-c2ccc3c(NS(=O)(=O)C4CCCCC4)ncnn23)cc2cnc(N[C@H]3CCCNC3)nc12] |
| I-4 | 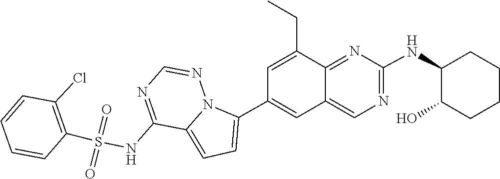 [SMILES: CCc1cc(-c2ccc3c(NS(=O)(=O)c4ccccc4Cl)ncnn23)cc2cnc(N[C@H]3CCCC[C@@H]3O)nc12] |
| I-5 | 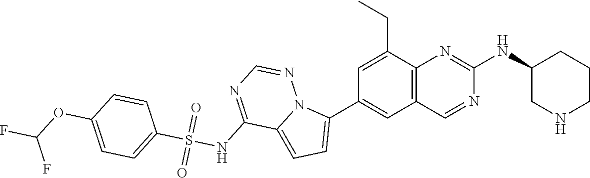 [SMILES: CCc1cc(-c2ccc3c(NS(=O)(=O)c4ccc(OC(F)F)cc4)ncnn23)cc2cnc(N[C@H]3CCCNC3)nc12] |
| I-6 | 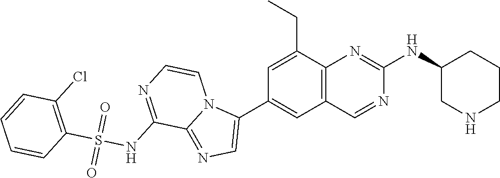 [SMILES: CCc1cc(-c2cnc3c(NS(=O)(=O)c4ccccc4Cl)nccn23)cc2cnc(N[C@H]3CCCNC3)nc12] |
| I-7 | 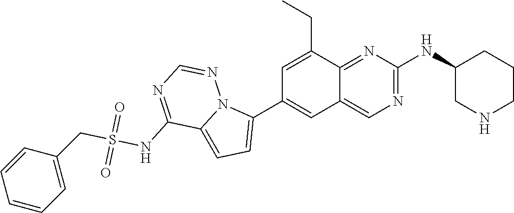 [SMILES: CCc1cc(-c2ccc3c(NS(=O)(=O)Cc4ccccc4)ncnn23)cc2cnc(N[C@H]3CCCNC3)nc12] |
| I-8 | 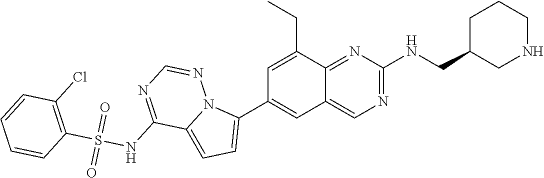 [SMILES: CCc1cc(-c2ccc3c(NS(=O)(=O)c4ccccc4Cl)ncnn23)cc2cnc(NC[C@H]3CCCNC3)nc12] |
| I-9 | 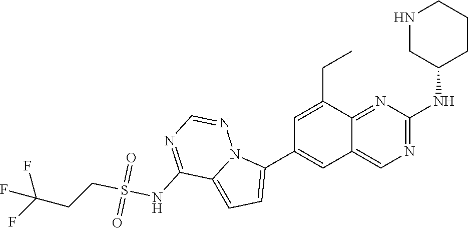 [SMILES: CCc1cc(-c2ccc3c(NS(=O)(=O)CCC(F)(F)F)ncnn23)cc2cnc(N[C@H]3CCCNC3)nc12] |
| I-10 | 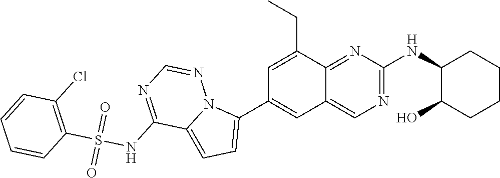 [SMILES: CCc1cc(-c2ccc3c(NS(=O)(=O)c4ccccc4Cl)ncnn23)cc2cnc(N[C@H]3CCCC[C@H]3O)nc12] |
| I-11 | 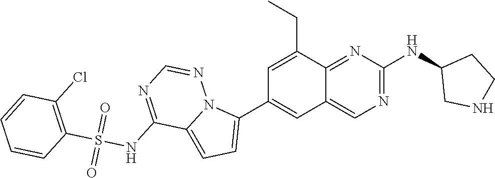 [SMILES: CCc1cc(-c2ccc3c(NS(=O)(=O)c4ccccc4Cl)ncnn23)cc2cnc(N[C@H]3CCNC3)nc12] |
| I-12 | 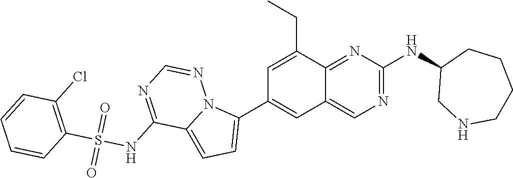 [SMILES: CCc1cc(-c2ccc3c(NS(=O)(=O)c4ccccc4Cl)ncnn23)cc2cnc(N[C@H]3CCCCNC3)nc12] |
| I-13 | 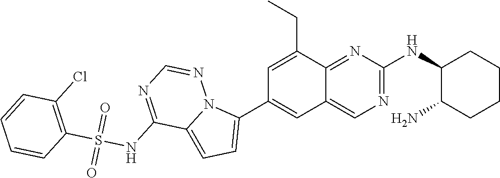 [SMILES: CCc1cc(-c2ccc3c(NS(=O)(=O)c4ccccc4Cl)ncnn23)cc2cnc(N[C@H]3CCCC[C@@H]3N)nc12] |
| I-14 | 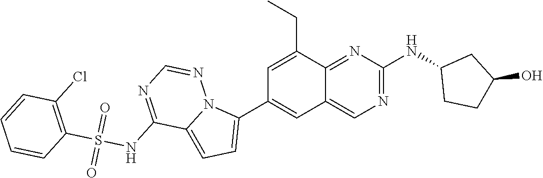 [SMILES: CCc1cc(-c2ccc3c(NS(=O)(=O)c4ccccc4Cl)ncnn23)cc2cnc(N[C@H]3CC[C@H](O)C3)nc12] |
| I-15 | 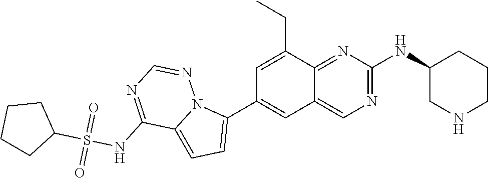 [SMILES: CCc1cc(-c2ccc3c(NS(=O)(=O)C4CCCC4)ncnn23)cc2cnc(N[C@H]3CCCNC3)nc12] |
| I-16 | 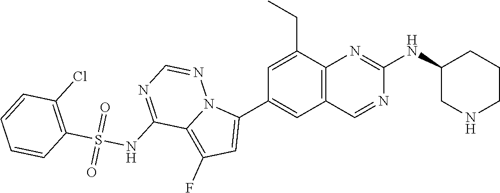 [SMILES: CCc1cc(-c2cc(F)c3c(NS(=O)(=O)c4ccccc4Cl)ncnn23)cc2cnc(N[C@H]3CCCNC3)nc12] |
| I-17 | 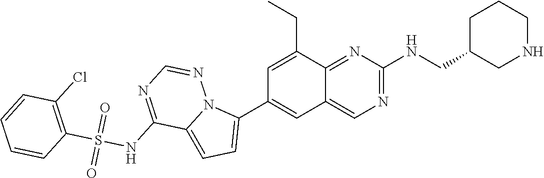 [SMILES: CCc1cc(-c2ccc3c(NS(=O)(=O)c4ccccc4Cl)ncnn23)cc2cnc(NC[C@@H]3CCCNC3)nc12] |
| I-18 | 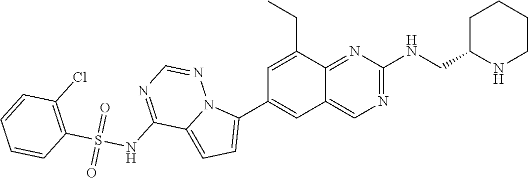 [SMILES: CCc1cc(-c2ccc3c(NS(=O)(=O)c4ccccc4Cl)ncnn23)cc2cnc(NC[C@@H]3CCCCN3)nc12] |
| I-19 | 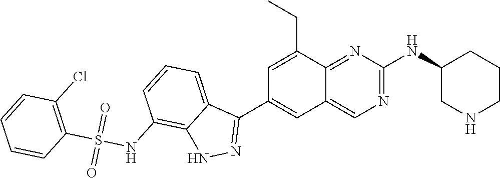 [SMILES: CCc1cc(-c2n[nH]c3c(NS(=O)(=O)c4ccccc4Cl)cccc23)cc2cnc(N[C@H]3CCCNC3)nc12] |
| I-20 | 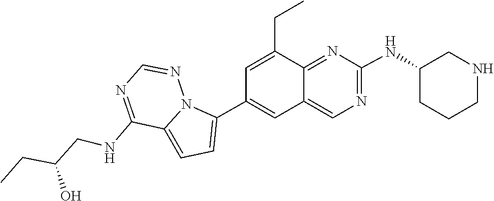 |
| I-21 | 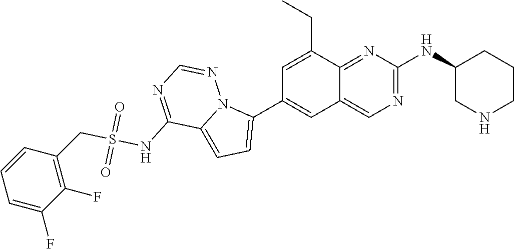 [SMILES: CCc1cc(-c2ccc3c(NS(=O)(=O)Cc4cccc(F)c4F)ncnn23)cc2cnc(N[C@H]3CCCNC3)nc12] |
| I-22 | 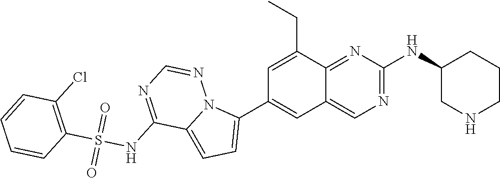 [SMILES: CCc1cc(-c2ccc3c(NS(=O)(=O)c4ccccc4Cl)ncnn23)cc2cnc(N[C@H]3CCCNC3)nc12] |
| I-23 | 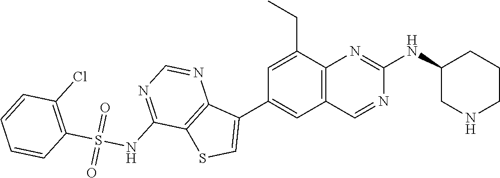 [SMILES: CCc1cc(-c2csc3c(NS(=O)(=O)c4ccccc4Cl)ncnc23)cc2cnc(N[C@H]3CCCNC3)nc12] |
| I-24 | 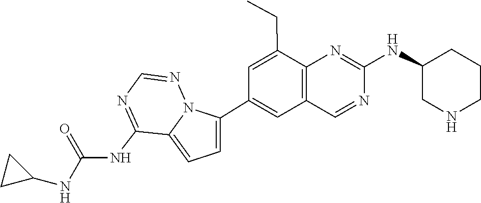 [SMILES: CCc1cc(-c2ccc3c(NC(=O)NC4CC4)ncnn23)cc2cnc(N[C@H]3CCCNC3)nc12] |
| I-25 | 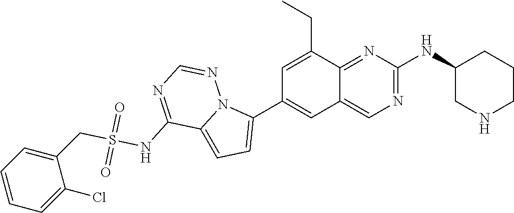 [SMILES: CCc1cc(-c2ccc3c(NS(=O)(=O)Cc4ccccc4Cl)ncnn23)cc2cnc(N[C@H]3CCCNC3)nc12] |
| I-26 | 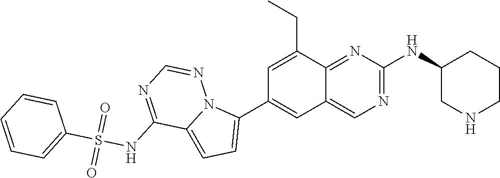 [SMILES: CCc1cc(-c2ccc3c(NS(=O)(=O)c4ccccc4)ncnn23)cc2cnc(N[C@H]3CCCNC3)nc12] |
| I-27 | 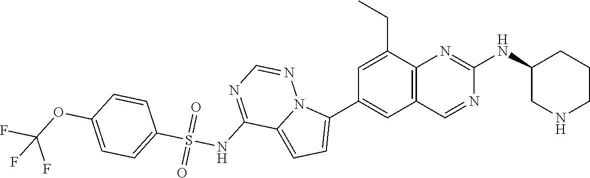 [SMILES: CCc1cc(-c2ccc3c(NS(=O)(=O)c4ccc(OC(F)(F)F)cc4)ncnn23)cc2cnc(N[C@H]3CCCNC3)nc12] |
| I-28 | 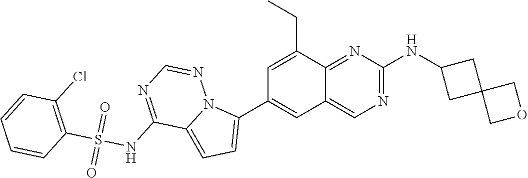 [SMILES: CCc1cc(-c2ccc3c(NS(=O)(=O)c4ccccc4Cl)ncnn23)cc2cnc(NC3CC4(COC4)C3)nc12] |
| I-29 | 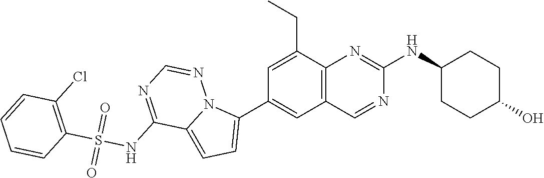 [SMILES: CCc1cc(-c2ccc3c(NS(=O)(=O)c4ccccc4Cl)ncnn23)cc2cnc(N[C@H]3CC[C@H](O)CC3)nc12] |
| I-30 | 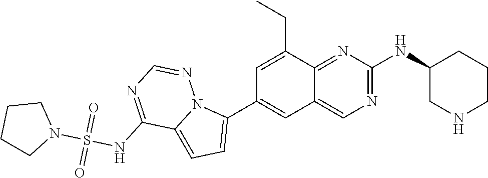 [SMILES: CCc1cc(-c2ccc3c(NS(=O)(=O)N4CCCC4)ncnn23)cc2cnc(N[C@H]3CCCNC3)nc12] |
| I-31 | 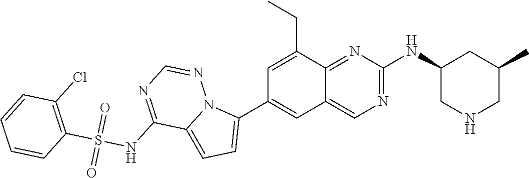 [SMILES: CCc1cc(-c2ccc3c(NS(=O)(=O)c4ccccc4Cl)ncnn23)cc2cnc(N[C@@H]3CNC[C@H](C)C3)nc12] |
| I-32 | 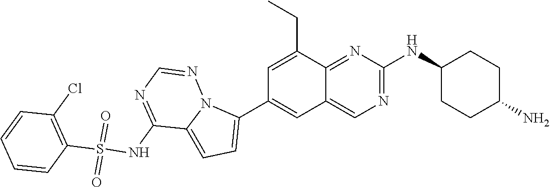 |
| I-33 | 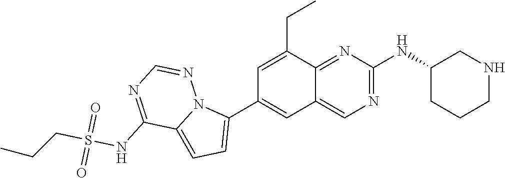 [SMILES: CCCS(=O)(=O)Nc1ncnn2c(-c3cc(CC)c4nc(N[C@H]5CCCNC5)ncc4c3)ccc12] |
| I-34 | 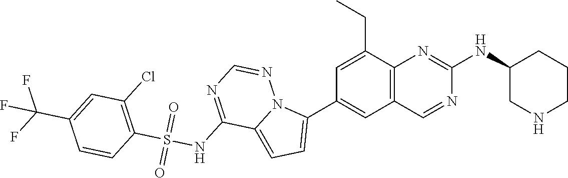 [SMILES: CCc1cc(-c2ccc3c(NS(=O)(=O)c4ccc(C(F)(F)F)cc4Cl)ncnn23)cc2cnc(N[C@H]3CCCNC3)nc12] |

In some embodiments, the present invention provides a compound set forth in Table 1, above, or a pharmaceutically acceptable salt thereof. In some embodiments, the present invention provides a compound set forth in Table 1, above. In some embodiments, the present invention provides a pharmaceutical composition comprising a compound set forth in Table 1 above, or a pharmaceutically acceptable salt thereof, together with a pharmaceutically acceptable carrier, excipient, or diluent.

In some embodiments, the present invention provides a compound as defined above, or a pharmaceutically acceptable salt thereof, or a pharmaceutical composition comprising a compound as defined above, or a pharmaceutically acceptable salt thereof, and a pharmaceutically acceptable carrier, adjuvant, or vehicle for use as a medicament.

In some embodiments, the invention also provides compounds described herein or pharmaceutical compositions described herein for use in a method for modulating IRE1α as described herein, in a method for enhancing an immune response in a subject in need thereof as described herein and/or in a method for treating a IRE1α-dependent disorder as described herein.

In some embodiments, the invention also provides compounds of described herein or pharmaceutical compositions described herein for use in a method for modulating IRE1α as described herein.

In some embodiments, the invention also provides compounds described herein or pharmaceutical compositions described herein for use in a method for inhibiting IRE1α as described herein, in a method for enhancing an immune response in a subject in need thereof as described herein and/or in a method for treating a IRE1α-dependent disorder as described herein.

In some embodiments, the invention also provides compounds of described herein or pharmaceutical compositions described herein for use in a method for inhibiting IRE1α as described herein.

In some embodiments, the invention also provides compounds as described herein or pharmaceutical compositions described herein for use in a method for enhancing IRE1α as described herein, in a method for enhancing an immune response in a subject in need thereof as described herein and/or in a method for treating a IRE1α-dependent disorder as described herein.

In some embodiments, the invention also provides compounds of described herein or pharmaceutical compositions described herein for use in a method for enhancing IRE1α as described herein.

In some embodiments, the invention also provides compounds as described herein or pharmaceutical compositions described herein for use in a method for enhancing an immune response in a subject in need thereof as described herein.

In some embodiments, the invention also provides compounds as described herein or pharmaceutical compositions described herein for use in a method for treating a IRE1α-dependent disorder as described herein.

In some embodiments, the invention also provides the use of a compound as described herein or a pharmaceutical composition described herein for the manufacture of a medicament for modulating IRE1α, a medicament for enhancing an immune response in a subject in need thereof and/or a medicament for treating a IRE1α-dependent disorder.

In some embodiments, the invention also provides the use of a compound as described herein or a pharmaceutical composition described herein for the manufacture of a medicament for inhibiting IRE1α.

In some embodiments, the invention also provides the use of a compound as described herein or a pharmaceutical composition described herein for the manufacture of a medicament for enhancing IRE1α.

In some embodiments, the invention also provides the use of a compound as described herein or a pharmaceutical composition described herein for the manufacture of a medicament for enhancing an immune response in a subject in need thereof.

In some embodiments, the invention also provides the use of a compound as described herein or a pharmaceutical composition described herein for the manufacture of a medicament treating a IRE1α-dependent disorder.

In some embodiments, the invention also provides the use of compounds as described herein or pharmaceutical compositions described herein in a method for inhibiting IRE1α as described herein, in a method for enhancing an immune response in a subject in need thereof as described herein and/or in a method for treating a IRE1α-dependent disorder as described herein.

In some embodiments, the invention also provides the use of compounds as described herein or pharmaceutical compositions described herein in a method for inhibiting IRE1α as described herein.

In some embodiments, the invention also provides the use of compounds as described herein or pharmaceutical compositions described herein in a method for enhancing IRE1α as described herein, in a method for enhancing an immune response in a subject in need thereof as described herein and/or in a method for treating a IRE1α-dependent disorder as described herein.

In some embodiments, the invention also provides the use of compounds as described herein or pharmaceutical compositions described herein in a method for enhancing IRE1α as described herein.

In some embodiments, the invention also provides the use of compounds as described herein or pharmaceutical compositions described herein in a method for enhancing an immune response in a subject in need thereof as described herein.

In some embodiments, the invention also provides the use of compounds as described herein or pharmaceutical compositions described herein in a method for treating a IRE1α-dependent disorder as described herein.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably modulate IRE1α, or a mutant thereof, in a biological sample or in a patient. In certain embodiments, the amount of compound in compositions of this invention is such that is effective to measurably modulate IRE1α, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or a modulatory active metabolite or residue thereof.

As used herein, the term “modulatory active metabolite or residue thereof” means that a metabolite or residue thereof is also a modulator of IRE1α, or a mutant thereof.

The subject matter disclosed herein includes prodrugs, metabolites, derivatives, and pharmaceutically acceptable salts of compounds of the invention. Metabolites include compounds produced by a process comprising contacting a compound of the invention with a mammal for a period of time sufficient to yield a metabolic product thereof. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

A compound of the invention can be in the form of a “prodrug,” which includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the invention are prodrugs of any of the formulae herein.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Activation of IRE1α's RNase is mediated by transautophosphorylation of the kinase domain (Tirasophon, W. et al. Genes Dev 12, 1812-1824 (1998)). An allosteric relationship between these two domains exists, which allows nucleotides (ADP and ATP) and small molecule inhibitors that stabilize an active ATP-binding site conformation to directly activate the RNase without autophosphorylation (Papa, F. R. et al. Science 302, 1533-1537 (2003); Han, D. et al. Biochemical and biophysical research communications 365, 777-783, (2008); Korennykh, A. V. et al. BMC biology 9, 48, (2011)). Conversely, type II kinase inhibitors stabilize an inactive ATP-binding site conformation of IRE1α and potently inhibit the RNase activity by breaking high-order oligomerization state (Wang, L. et al. Nature chemical biology 8, 982-989, (2012)). These compounds are kinase-inhibiting RNase-attenuators (KIRAs).

Distinct classes of ATP-competitive kinase inhibitors divergently modulate the RNase activity of IRE1α. A co-crystal structure of yeast IRE1 bound with APY29, a predicted type I kinase inhibitor, shows that the kinase catalytic domain is in an active conformation, which is a conformation typically adopted by protein kinases when bound to ATP and other type I inhibitors (Korennykh, A. V. et al. Nature 457, 687-693 (2009); Korennykh, A. V. et al. BMC Biol. 9, 48 (2011)). Moreover, two additional co-crystal structures of yeast IRE1 and human IRE1α bound with ADP show that the kinase domain is similarly in an active conformation (Ali, M. M. et al. EMBO J. 30, 894-905 (2011); Lee, K. P. et al. Cell 132, 89-100 (2008)). By stabilizing IRE1α 's kinase in the active conformation, these type I inhibitors act as ligands that allosterically activate its adjacent RNase domain. It might be possible to stabilize IRE1α 's kinase domain in an alternative conformation, and in so doing, disable its RNase activity. Use of a class of small molecule kinase inhibitors that have been described to selectively stabilize the inactive conformation of the ATP-binding site (type II inhibitors) for a variety of kinases; examples include the clinically-approved drugs imatinib and sorafenib (Liu, Y. & Gray, N. S. Nat. Chem. Biol. 2, 358-364 (2006); Wan, P. T. et al. Cell 116, 855-867 (2004); Schindler, T. et al. Science 289, 1938-1942 (2000)), provides support for this approach. The inactive ATP-binding site conformation stabilized by type II inhibitors is characterized by outward movement of the catalytically-important Asp-Phe-Gly (DFG) motif, and is therefore called the DFG-out conformation (Liu, Y. & Gray, N. S. Nat. Chem. Biol. 2, 358-364 (2006); Ranjitkar, P. et al. Chem. Biol. 17, 195-206 (2010)). In contrast, in all three co-crystal structures of IRE1 in an active conformation mentioned previously, the kinase domain adopts the DFG-in conformation (Korennykh, A. V. et al. Nature 457, 687-693 (2009); Ali, M. M. et al. EMBOJ. 30, 894-905 (2011); Lee, K. P. et al. Cell 132, 89-100 (2008)).

Under high endoplasmic reticulum (ER) stress, hyperactivation of intracellular signaling pathways, termed the unfolded protein response (UPR), triggers cell death. Signature events of this terminal UPR are controlled by IRE1α, an ER bifunctional kinase/endoribonuclease (RNase), which, when oligomerized, endonucleolytically degrades ER-localized mRNAs and repressive micro-RNA precursors to trigger apoptosis. IRE1α somatic mutations found in human cancers disable oligomerization and apoptotic function of its RNase. Using these instructive results from human biology, ATP-competitive KIRAs were developed that allosterically reduce IRE1α oligomerization and RNase activity. One such kinase inhibitor, KIRA6, inhibits all IRE1α outputs, and preserves cell viability and function under ER stress. In rat models of retinal degeneration caused by ER stress, intravitreal KIRA6 prevents photoreceptor loss.

In some embodiments, a compound disclosed herein selectively binds to an isoform of IRE1. In some embodiments, the IRE1 protein is human IRE1α.

In some embodiments, a compound disclosed herein selectively binds to an IRE1 isoform comprising a kinase domain and/or an RNase domain. In some embodiments, the kinase domain is a trans-autophosphorylation kinase domain. In some embodiments, the IRE1 family protein is IRE1α.

In some embodiments, a compound disclosed herein selectively binds to an ATP-binding pocket within a trans-autophosphorylation kinase domain region of IRE1α.

In some embodiments, a compound disclosed herein selectively binds to an activation loop within a trans-autophosphorylation kinase domain region of IRE1α.

In some embodiments, a compound disclosed herein selectively binds to an RNase domain region of IRE1α.

In some embodiments, a compound disclosed herein selectively binds to a kinase domain dimer interface amino acid residue.

In some embodiments, a compound disclosed herein selectively binds to a first IRE1α and blocks dimerization between kinase domain dimer interface amino acid residues of the first IRE1α and a second IRE1α.

In some embodiments, a compound disclosed herein selectively binds to a kinase-extension nuclease (KEN) domain dimer interface amino acid residue of IRE1α.

In some embodiments, a compound disclosed herein selectively binds to an RNase domain and a trans-autophosphorylation kinase domain region of IRE1α. In some embodiments, a compound disclosed herein selectively binds to an RNase domain and an ATP-binding pocket within a trans-autophosphorylation kinase domain region of IRE1α. In some embodiments, a compound disclosed herein selectively binds to an RNase domain and an activation loop within a trans autophosphorylation kinase domain region of IRE1α.

In some embodiments, a compound disclosed herein selectively binds to IRE1α at two sites located in an RNase domain, trans-autophosphorylation kinase domain region, ATP-binding pocket, activation loop, or any combination thereof. In some embodiments, a compound disclosed herein selectively binds to IRE1α at two or more sites. In some embodiments, a compound disclosed herein selectively binds to IRE1α at two or more sites located in an RNase domain, trans-autophosphorylation kinase domain region, ATP-binding pocket, activation loop, or any combination thereof. In some embodiments, a compound disclosed herein selectively binds to IRE1α at three sites located in an RNase domain, trans-autophosphorylation kinase domain region, ATP-binding pocket, activation loop, or any combination thereof.

In some embodiments, a compound disclosed herein selectively binds to IRE1α at a first site located in an RNase domain, trans-autophosphorylation kinase domain region, ATP-binding pocket, or activation loop. In some embodiments, a compound disclosed herein selectively binds to IRE1α at a second site located in an RNase domain, trans-autophosphorylation kinase domain region, ATP-binding pocket, or activation loop. In some examples, the first site is located within the same domain or region as the second site. In some examples, the first site is located within a different domain or region as the second site.

In some embodiments, a compound disclosed herein selectively binds to first IRE1α, thereby blocking dimerization of the first IRE1α to a second IRE1α. In some embodiments, a compound disclosed herein selectively binds to first IRE1α, thereby blocking auto-transphosphorylation of the first IRE1α or a second IRE1α to which the first IRE1α is dimerized.

In some embodiments, a compound disclosed herein selectively binds to a first IRE1α, thereby blocking activation of the first IRE1α or a second IRE1α to which the first IRE1α is dimerized.

In some embodiments, a compound disclosed herein selectively binds to a first IRE1α, thereby blocking kinase activity of the first IRE1α or a second IRE1α to which the first IRE1α is dimerized. In some embodiments, a compound disclosed herein selectively binds to a first IRE1α, thereby blocking RNase activity of the first IRE1α or a second IRE1α to which the first IRE1α is dimerized.

In some embodiments, a compound disclosed herein selectively binds to IRE1α when in a homo-dimerized conformation. In some embodiments, a compound disclosed herein selectively binds to IRE1α when in an oligomerized conformation. In some embodiments, a compound disclosed herein selectively binds to IRE1α when in a non-oligomerized or non-dimerized conformation. In some embodiments, a compound disclosed herein selectively binds to IRE1α when in an ATP-bound state. In some embodiments, a compound disclosed herein selectively binds to a IRE1 family protein when in a non-ATP-bound state. In some embodiments, the compound is a pharmaceutically acceptable salt, or solvate thereof.

### IRE1 Signaling Pathway

In some embodiments, a compound disclosed herein selectively binds to an IRE1 protein and alters a downstream signaling pathway. In some embodiments, a compound disclosed herein selectively binds to an IRE1 protein and alters signaling of immunoglobulin heavy-chain binding protein (BIP), protein kinase R (PKR)-like endoplasmic reticulum kinase (PERK), glucose regulate protein 78 (Grp78), eukaryotic translation initiation factor 2α (eIF2α), X-box binding protein 1 (XBP1), activating transcription factor 6α (ATF6α), C/EBP homologous protein (CHOP), growth arrest and DNA damage-inducible protein 34 (GADD34), tumor necrosis factor receptor-associated factor 2 (TRAF2), JUN N-terminal kinase (INK), regulated IRE1-dependent decay (RIDD), transcriptionally active XBP1 (XBP1s), or unspliced XBP1 (XBP1u). In some embodiments, a compound disclosed herein selectively binds to an IRE1 protein and alters a downstream cellular process.

In some embodiments, a compound disclosed herein selectively binds to an IRE1 protein and decreases or blocks a downstream signaling pathway. In some embodiments, a compound disclosed herein selectively binds to an IRE1 protein and decreases or blocks activity or signaling of TXNIP, Caspase 1, Interleukin 1-β, INK, Bim, cytochrome C, Caspase 3, Caspase 8, mRNA degradation, miRNA degradation, apoptotosis-inducing proteins, or inflammation-inducing proteins. In some embodiments, a compound disclosed herein selectively binds to an IRE1 protein and decreases XBP1 mRNA levels. In some embodiments, a compound disclosed herein selectively binds to an IRE1 protein and decreases transcriptionally active XBP1 (XBP1s) mRNA levels. In some embodiments, a compound disclosed herein selectively binds to an IRE1 protein and decreases spliced XBP1 mRNA levels. In some embodiments, an IRE1 protein is IRE1, IRE1α or ERN1.

In some embodiments, a compound disclosed herein selectively binds to an IRE1 protein and increases, activates, or removes a block of a downstream signaling pathway. In some embodiments, a compound disclosed herein selectively binds to an IRE1 protein and increases, activates, or removes a block of activity or signaling of Bcl2, Bcl-XL, Mcl-1, Bax, Bak, other anti-apoptotic proteins, or an mRNA translocon proteins.

In some embodiments, a compound disclosed herein selectively binds to an IRE1 protein and disrupts binding with an effector protein. In some cases, the effector protein binds to the IRE1 protein when in a dimerized or oligomerized state. In some cases, the effector protein binds to the IRE1 protein when in a non-dimerized or non-oligomerized state. In some cases, the effector protein is immunoglobulin heavy-chain binding protein (BIP), protein kinase R (PKR)-like endoplasmic reticulum kinase (PERK), glucose regulate protein 78 (Grp78), tumor necrosis factor receptor-associated factor 2 (TRAF2), JUN N-terminal kinase (INK), transcriptionally active XBP1 (XBP1s), unspliced XBP1 (XBP1u), regulated IRE1-dependent decay (RIDD), Heat shock protein 90 kDa alpha (HSP 90-alpha), or misfolded protein. In some embodiments, an IRE1 protein is IRE1α or IRE1β.

In some embodiments, a compound disclosed herein selectively binds to an IRE1 protein and alters activity of a cellular process or cellular function, such as regulated IRE1-dependent decay (RIDD), RNA decay, translation, autophagy, cell survival, ER protein folding, ERAD, reactive oxygen species generation, transport, ER-associated protein degradation (ERAD), protein synthesis, or apoptosis. In some embodiments, where an altered or lack of a cellular process or cellular function is associated with a disease state, selective binding of a compound disclosed herein results in inhibiting or alleviating the disease state, or inhibiting a deleterious activity associated with the disease state. In some embodiments, an IRE1 protein is IRE1α or IRE1β.

### Methods of Treatment

In one aspect is provided a method of modulating the activity of an IRE1 (e.g. IRE1α) protein, the method including contacting the IRE1α protein with an effective amount of a compound described herein, or a pharmaceutically acceptable salt thereof.

In certain embodiments, the modulating is inhibiting. In some embodiments, the activity is kinase activity. In some embodiments, the kinase activity is autophosphorylation activity. In various embodiments, the kinase activity is trans-autophosphorylation activity. In various embodiments, the activity is oligomerization activity. In certain embodiments, the oligomerization activity is dimerization activity. In some embodiments, the activity is RNase activity. In various embodiments, the activity is miR cleavage. In various embodiments, the activity is miR-17 cleavage. In certain embodiments, the activity is miR-34a cleavage. In some embodiments, the activity is miR-96 cleavage. In some embodiments, the activity is miR-125b cleavage. In various embodiments, the activity is XBP1 mRNA splicing. In some embodiments, the activity is UPR activation. In various embodiments, the activity is terminal UPR activation. In some embodiments, a cell includes the IRE1α protein. In various embodiments, the activity of the IRE1α protein is increasing apoptosis of the cell. In some embodiments, an organ includes the cell. In certain embodiments, an organism includes the cell. In some embodiments, an organism has a disease associated with the IRE1α protein activity.

In certain embodiments, the modulating is activating. In certain embodiments, the modulating is inhibiting.

In one aspect, the invention provides for a method of treating a patent suffering from an IRE1-related disease or disorder, comprising administering to a patient a therapeutically effective amount of a compound of the invention, or pharmaceutically acceptable salt thereof. In another aspect, the method comprises administering to a patient with an IRE1-related disease or condition, an effective amount of a pharmaceutical composition comprising a compound of the invention, or pharmaceutically acceptable salt thereof, and a pharmaceutically acceptable carrier, glidant, diluent, or excipient. In some embodiments, the patient is a human patient.

In some embodiments, a compound disclosed herein is used to treat or ameliorate a disease associated with altered IRE1α pathway signaling when administered to a subject in need thereof. In some cases, a compound disclosed herein is used to treat or ameliorate the effects of a disease associated with altered IRE1α pathway signaling when administered to a subject in need thereof.

In one embodiment, the disease or disorder arises from abnormal cell growth, function or behavior associated with the UPR pathway such as cancer, an immune disorder, cardiovascular disease, viral infection, inflammation, a metabolism/endocrine disorder or a neurological disorder. In certain embodiments, the disease or disorder is selected from neurodegenerative diseases, inflammation, metabolic disorders, liver dysfunction, brain ischemia, heart ischemia, autoimmune diseases, and cancer.

In one embodiment, the disease is a neurodegenerative disease, demyelinating disease, eye disease, fibrotic disease, or diabetes.

In various embodiments, the disease is a neurodegenerative disease. In embodiments, the neurodegenerative disease is retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru. In some embodiments, the disease is amyotrophic lateral sclerosis. In some embodiments, the disease is retinal degeneration. In certain embodiments, the disease is retinitis pigmentosa.

In certain embodiments, the disease is a demyelinating disease. In some embodiments, the demyelinating disease is Wolfram Syndrome, Pelizaeus-Merzbacher Disease, Transverse Myelitis, Charcot-Marie-Tooth Disease, or Multiple Sclerosis. In various embodiments, the disease is Multiple Sclerosis.

In certain embodiments, the disease is diabetes. In various embodiments, the diabetes is type I diabetes. In some embodiments, the diabetes is type II diabetes.

In some embodiments, the disease is an eye disease. In certain embodiments, the eye disease is retinitis pigmentosa. In various embodiments, the eye disease is retinal degeneration. In some embodiments, the eye disease is macular degeneration. In various embodiments, the eye disease is Wolfram Syndrome. In certain embodiments, the disease is idiopathic pulmonary fibrosis (IPF).

In certain embodiments, the disease is a fibrotic disease. In some embodiments, the fibrotic disease is idiopathic pulmonary fibrosis (IPF), myocardial infarction, cardiac hypertrophy, heart failure, kidney fibrosis, cirrhosis, acetominophen liver toxicity, hepatitis C liver disease, hepatosteatosis (fatty liver disease), or hepatic fibrosis. In some embodiments, the disease is interstitial lung disease (ILD). In some embodiments, the disease is myocardial infarction. In some embodiments, the disease is cardiac hypertrophy. In some embodiments, the disease is heart failure. In some embodiments, the disease is cirrhosis. In certain embodiments, the disease is acetominophen liver toxicity. In certain embodiments, the disease is hepatitis C liver disease. In some embodiments, the disease is hepatosteatosis (fatty liver disease). In some embodiments, the disease is hepatic fibrosis.

In one aspect, the invention provides a method of treating a cell proliferation disorder in a subject, comprising administering a therapeutically effective amount of a compound of the invention, or a pharmaceutically acceptable salt, hydrate, solvate, or prodrug thereof, to the subject.

In one aspect, the invention provides for a method of treating a patent suffering from cancer, the method comprising administration of a compound of the present invention. In certain embodiments, the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, pancreatic, myeloid disorders, lymphoma, hairy cells, buccal cavity, naso-pharyngeal, pharynx, lip, tongue, mouth, small intestine, colon-rectum, large intestine, rectum, brain and central nervous system, Hodgkin's, leukemia, bronchus, thyroid, liver and intrahepatic bile duct, hepatocellular, gastric, glioma/glioblastoma, endometrial, melanoma, kidney and renal pelvis, urinary bladder, uterine corpus, uterine cervix, multiple myeloma, acute myelogenous leukemia, chronic myelogenous leukemia, lymphocytic leukemia, chronic lymphoid leukemia (CLL), myeloid leukemia, oral cavity and pharynx, non-Hodgkin lymphoma, melanoma, and villous colon adenoma.

In some embodiments, the disease or disorder is cancer selected from the group consisting of squamous cell cancer, small-cell lung cancer, non-small cell lung cancer (“NSCLC”), adenocarcinoma of the lung and squamous carcinoma of the lung, cancer of the peritoneum, hepatocellular cancer, stomach cancer, gastrointestinal cancer, esophageal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

In some embodiments, the cancer is multiple myeloma. In some embodiments, the cancer is a breast cancer (e.g., a triple-negative breast cancer (TNBC)).

In certain embodiments, the cancer comprises at least one cancer selected from the group consisting of colorectal cancer, melanoma, non-small cell lung cancer, ovarian cancer, breast cancer, pancreatic cancer, a hematological malignancy, and a renal cell carcinoma.

In certain aspects, the invention provides a method of treating cell proliferation disorders, including cancers, benign papillomatosis, gestational trophoblastic diseases, and benign neoplastic diseases, such as skin papilloma (warts) and genital papilloma.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In certain embodiments, the cancer is brain cancer, leukemia, skin cancer, prostate cancer, thyroid cancer, colon cancer, lung cancer or sarcoma. In another embodiment the cancer is selected from the group consisting of glioma, glioblastoma multiforme, paraganglioma, suprantentorial primordial neuroectodermal tumors, acute myeloid leukemia, myelodysplastic syndrome, chronic myelogenous leukemia, melanoma, breast, prostate, thyroid, colon, lung, central chondrosarcoma, central and periosteal chondroma tumors, fibrosarcoma, and cholangiocarcinoma.

In a further embodiment, the cancer is selected from brain and spinal cancers. In particular embodiments, the cancer is selected from the group consisting of anaplastic astrocytomas, glioblastomas, astrocytomas, and estheosioneuroblastomas (olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET).

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CMIL), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers.

In specific embodiments, the cancer is selected from cancers of the reproductive system. In particular embodiments, the cancer is selected from the group consisting of breast cancers, cervical cancers, vaginal cancers, ovarian cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the cancer is selected from liver and bile duct cancers. In particular embodiments, the cancer is liver cancer (hepatocellular carcinoma). In particular embodiments, the cancer is bile duct cancer (cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urethelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, and chordoma.

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, and primary central nervous system lymphomas.

In specific embodiments, the cancer is selected from glandular cancers. In particular embodiments, the cancer is selected from the group consisting of adrenocortical cancer, pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosacroma, angiosarcoma, chondrosarcoma, infantile fibrosarcoma, and synovial sarcoma.

In specific embodiments, the cell-proliferation disorder is selected from benign papillomatosis, benign neoplastic diseases and gestational trophoblastic diseases. In particular embodiments, the benign neoplastic disease is selected from skin papilloma (warts) and genital papilloma. In particular embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

In some embodiments, the subject has melanoma. The melanoma may be at early stage or at late stage. In some embodiments, the subject has colorectal cancer. The colorectal cancer may be at early stage or at late stage. In some embodiments, the subject has non-small cell lung cancer. The non-small cell lung cancer may be at early stage or at late stage. In some embodiments, the subject has pancreatic cancer. The pancreatic cancer may be at early stage or late state. In some embodiments, the subject has a hematological malignancy. The hematological malignancy may be at early stage or late stage. In some embodiments, the subject has ovarian cancer. The ovarian cancer may be at early stage or at late stage. In some embodiments, the subject has breast cancer. The breast cancer may be at early stage or at late stage. In some embodiments, the subject has renal cell carcinoma. The renal cell carcinoma may be at early stage or at late stage. In some embodiments, the cancer has elevated levels of T-cell infiltration.

In some embodiments, cancers treatable with compounds of the present disclosure include melanoma (e.g., metastatic malignant melanoma), renal cancer (e.g. clear cell carcinoma), prostate cancer (e.g. hormone refractory prostate adenocarcinoma), breast cancer, triple-negative breast cancer, colon cancer and lung cancer (e.g. non-small cell lung cancer and small cell lung cancer). Additionally, the disclosure includes refractory or recurrent malignancies whose growth may be inhibited using the compounds of the disclosure.

In some embodiments, cancers treatable with compounds of the present disclosure include hematological cancers including lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET)), myelodysplasia syndrome (MDS), T-cell acute lymphoblastic lymphoma (T-ALL), multiple myeloma, cutaneous T-cell lymphoma, Waldenstrom's Macroglubulinemia, hairy cell lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers.

In some embodiments, the cancer is ovarian cancer, breast cancer, or triple negative breast cancer (TNBC).

In some embodiments, the cancer includes tumors with high levels of MYC that drives upregulated unfolded protein response.

In some instances, a compound disclosed herein is used to reinforce anti-tumor mechanisms. In some cases, an anti-tumor mechanism comprises direct inhibition of tumor growth. In some cases, an anti-tumor mechanism comprises induction of anti-tumor immunity. In some cases, anti-tumor mechanisms comprise direct inhibition of tumor growth and simultaneous induction of anti-tumor immunity. In some cases, a compound disclosed herein can prevent lipid accumulation in myeloid cells exposed to ovarian cancer-derived ascites supernatants. In some cases, a compound disclosed herein can block myeloid cell immunosuppression mediated by tumor-associated factors. In some cases, a compound disclosed herein can be employed as therapeutic compound that enhances dendritic cell and T cell anti-tumor activity in mammals. For example, the compounds disclosed herein can be used to treat murine and human ovarian cancers.

Also provided herein is a method of treating a disease caused by abnormal levels of IRE1 activity in a human or animal patient in need of such treatment with a compound of the invention. The disease may be caused by an amount of IRE1 activity that is too low or too high. For example, the disease may be caused by a deficiency in IRE1 activity or by abnormally high IRE1 activity (e.g., hyperactivity of IRE1). The method includes administering to the patient a therapeutically effective amount of an IRE1 modulator disclosed herein.

IRE1 deficiency is a decreased amount of IRE1 activity compared to normal levels of IRE1 activity in a particular subject or a population of healthy subjects. The decreased amount of IRE1 activity results in excessive amounts of misfolded protein accumulation thereby causing the disease state. In some embodiments, the disease is associated with IRE1 deficiency. Such diseases include, but are not limited to, cystic fibrosis, retinitis pigmentosa, diabetes, or a neurodegenerative disease. The neurodegenerative disease may include Alexander's disease, Alper's disease, Alzheimer's disease, Amyotrophic lateral sclerosis, Ataxia telangiectasia, Batten disease (also known as Spielmeyer-Vogt-Sjogren-Batten disease). Bovine spongiform encephalopathy (BSF), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

IRE1 hyperactivity is an increased amount of IRE1 activity compared to normal levels of IRE1 activity in a particular subject or a population of healthy subjects. The increased amount of IRE1 activity may result in, for example, excessive amounts of cell proliferation thereby causing the disease state.

In other embodiments, the disease is associated with abnormally high IRE1. Such diseases include, but are not limited, to cancers, inflammatory diseases, and autoimmune diseases. Exemplary cancers include, but am not limited to, breast cancer and multiple myeloma. In one embodiment, the disease is multiple myeloma. In one embodiment, the disease is a triple-negative breast cancer. Exemplary inflammatory diseases include, but are not limited to, asthma, chronic inflammation, chronic prostatitis, glomerulonephritis, hypersensitivities, inflammatory bowel diseases, pelvic inflammatory disease; reperfusion injury, rheumatoid arthritis, transplant rejection, and vasculitis. Exemplary autoimmune diseases include, but are not limited to, XBP1-linked Crohn's disease, Coeliac disease, diabetes mellitus type 1 (IDDM), systemic lupus erythematosus (SLE), Sjogren's syndrome, Churg-Strauss Syndrome, Hashimoto's thyroiditis, Graves' disease, idiopathic thrombocytopenic purpura, and rheumatoid arthritis. In one embodiment, the disease is XBP1-linked Crohn's disease.

In some embodiments, the invention provides a pharmaceutical composition comprising an effective amount of a compound of the invention, or a pharmaceutically acceptable salt, hydrate, solvate, or prodrug thereof, and a pharmaceutically acceptable carrier.

The presently disclosed compounds may be administered in any suitable manner known in the art. In some embodiments, the compound of the invention or a pharmaceutically acceptable salt, prodrug, metabolite, or derivative thereof is administered intravenously, intramuscularly, subcutaneously, topically, orally, transdermally, intraperitoneally, intraorbitally, by implantation, by inhalation, intrathecally, intraventricularly, intratumorally, or intranasally.

In some embodiments, the compound of the invention is administered continuously. In other embodiments, the compound of the invention is administered intermittently. Moreover, treatment of a subject with an effective amount of a compound of the invention can include a single treatment or can include a series of treatments.

It is understood that appropriate doses of the active compound depends upon a number of factors within the knowledge of the ordinarily skilled physician or veterinarian. The dose(s) of the active compound will vary, for example, depending upon the age, body weight, general health, gender, and diet of the subject, the time of administration, the route of administration, the rate of excretion, and any drug combination.

It will also be appreciated that the effective dosage of a compound of the invention or a pharmaceutically acceptable salt, prodrug, metabolite, or derivative thereof used for treatment may increase or decrease over the course of a particular treatment. Changes in dosage may result and become apparent from the results of diagnostic assays.

In some embodiments, the compound of the invention is administered to the subject at a dose of between about 0.001 μg/kg and about 1000 mg/kg, including but not limited to about 0.001 μg/kg, 0.01 μg/kg, 0.05 μg/kg, 0.1 μg/kg, 0.5 μg/kg, 1 μg/kg, 10 μg/kg, 25 μg/kg, 50 μg/kg, 100 μg/kg, 250 μg/kg, 500 μg/kg, 1 mg/kg, 5 mg/kg, 10 mg/kg, 25 mg/kg, 50 mg/kg, 100 mg/kg, and 200 mg/kg.

In the methods described herein, the method can further comprise administering a chemotherapeutic agent to the subject. In certain aspects of this embodiment, the chemotherapeutic agent is administered to the subject simultaneously with the compound or the composition. In certain aspects of this embodiment, the chemotherapeutic agent is administered to the subject prior to administration of the compound or the composition. In certain aspects of this embodiment, the chemotherapeutic agent is administered to the subject after administration of the compound or the composition.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “subject” refers to animals such as mammals, including, but not limited to, primates (e.g., humans), cows, sheep, goats, horses, dogs, cats, rabbits, rats, mice and the like. In certain embodiments, the subject is a human.

The term “patient” refers to animals such as mammals, including, but not limited to, primates (e.g., humans), cows, sheep, goats, horses, dogs, cats, rabbits, rats, mice and the like. In certain embodiments, the patient is a human.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of formula I and one or more additional therapeutic agents. The therapeutic agent may be administered together with a compound of formula I, or may be administered prior to or following administration of a compound of formula I. Suitable therapeutic agents are described in further detail below. In certain embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a compound of formula I and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating rheumatoid arthritis comprising administering to a patient in need thereof a compound of formula I and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a compound of formula I and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating cutaneous lupus erythematosus or systemic lupus erythematosus comprising administering to a patient in need thereof a compound of formula I and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating Crohn's disease, ulcerative colitis, or inflammatory bowel disease comprising administering to a patient in need thereof a compound of formula I and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a compound of formula I and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a compound of formula I and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In another embodiment, the present invention provides a method of treating a hematological malignancy comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a solid tumor comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a hematological malignancy comprising administering to a patient in need thereof a compound of formula I and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a compound of formula I and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating or lessening the severity of a disease comprising administering to a patient in need thereof a compound of formula I and a BTK inhibitor, wherein the disease is selected from inflammatory bowel disease, arthritis, cutaneous lupus erythematosus, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, cutaneous lupus erythematosus, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a compound of formula I and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a compound of formula I and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments the present invention provides a method of treating or lessening the severity of a disease comprising administering to a patient in need thereof a compound of formula I and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukemia, diffuse large B-cell lymphoma, Hodgkin's disease, small-cell lung cancer, non-small-cell lung cancer, myelodysplastic syndrome, lymphoma, a hematological neoplasm, or solid tumor.

In some embodiments, the disease is an autoimmune disorder, an inflammatory disorder, a proliferative disorder, an endocrine disorder, a neurological disorder, or a disorder associated with transplantation. In some embodiments the JH2 binding compound is a compound of formula I. Other suitable JH2 domain binding compounds include those described in WO2014074660A1, WO2014074661A1, WO2015089143A1, the entirety of each of which is incorporated herein by reference. Suitable JH1 domain binding compounds include those described in WO2015131080A1, the entirety of which is incorporated herein by reference.

The compounds and compositions, according to the method of the present invention, may be administered using any amount and any route of administration effective for treating or lessening the severity of an autoimmune disorder, an inflammatory disorder, a proliferative disorder, an endocrine disorder, a neurological disorder, or a disorder associated with transplantation. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity in a biological sample comprising the step of contacting said biological sample with a compound of this invention, or a composition comprising said compound.

According to another embodiment, the invention relates to a method of inhibiting HPK1, or a mutant thereof, activity in a biological sample comprising the step of contacting said biological sample with a compound of this invention, or a composition comprising said compound. In certain embodiments, the invention relates to a method of irreversibly inhibiting HPK1, or a mutant thereof, activity in a biological sample comprising the step of contacting said biological sample with a compound of this invention, or a composition comprising said compound.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Inhibition of HPK1 (or a mutant thereof) activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of inhibiting protein kinase activity in a patient comprising the step of administering to said patient a compound of the present invention, or a composition comprising said compound.

According to another embodiment, the invention relates to a method of inhibiting activity of HPK1, or a mutant thereof, in a patient comprising the step of administering to said patient a compound of the present invention, or a composition comprising said compound. According to certain embodiments, the invention relates to a method of reversibly or irreversibly inhibiting one or more of HPK1, or a mutant thereof, activity in a patient comprising the step of administering to said patient a compound of the present invention, or a composition comprising said compound. In other embodiments, the present invention provides a method for treating a disorder mediated by HPK1, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™ Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, the present invention provides a method of treating AML associated with an ITD and/or D835Y mutation, comprising administering a compound of the present invention together with a one or more FLT3 inhibitors. In some embodiments, the FLT3 inhibitors are selected from quizartinib (AC220), a staurosporine derivative (e.g. midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID (TM) CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein. Additional compounds of the invention were prepared by methods substantially similar to those described herein in the Examples and methods known to one skilled in the art.

### Example 1: Synthesis of (S)—N-(7-(8-ethyl-2-(piperidin-3-ylamino)quinazolin-6-yl)pyrrolo[2,1-f][1,2,4]triazin-4-yl)-2-fluorobenzenesulfonamide. I-1

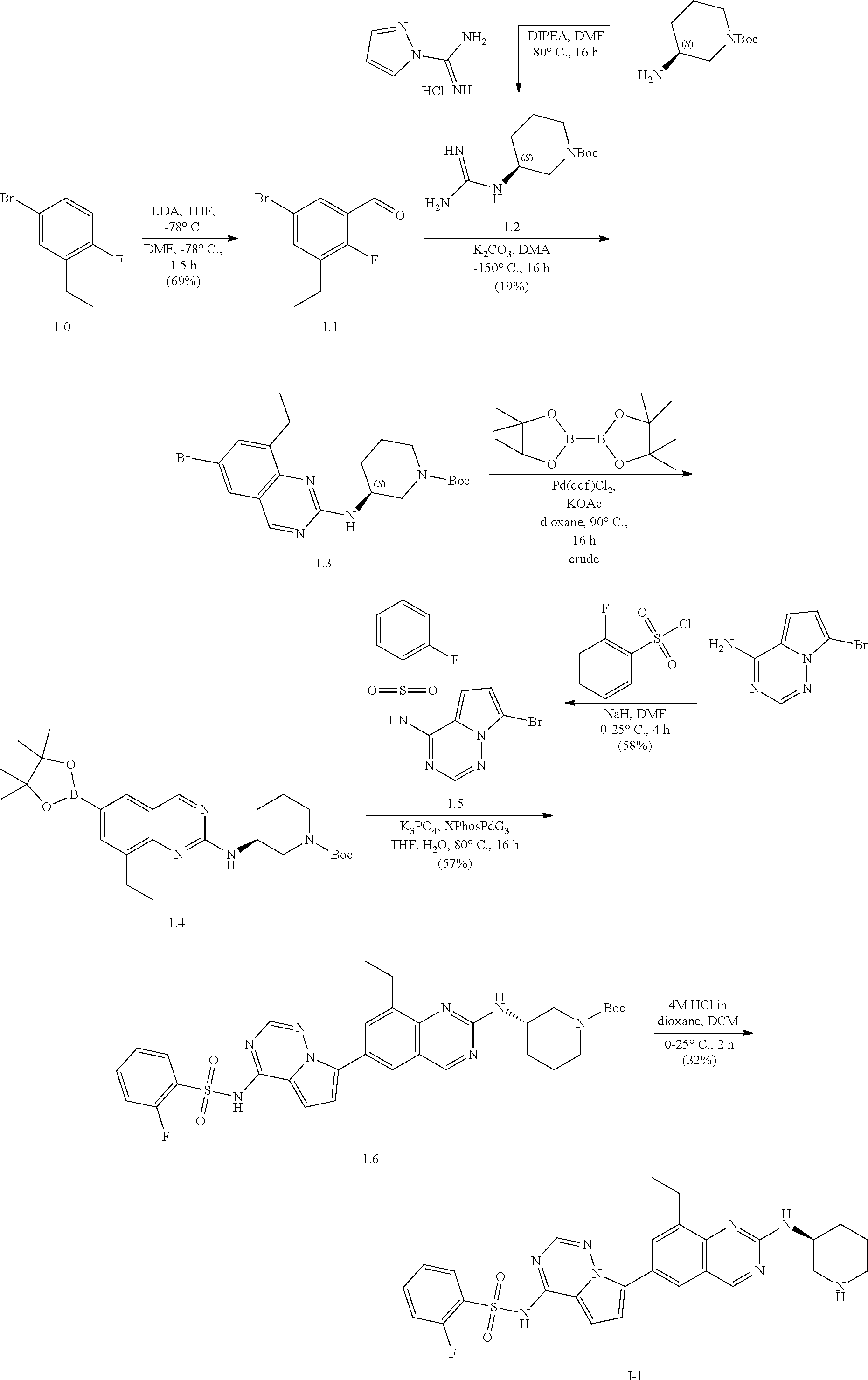

### Synthesis of 1.1.

To a stirred solution of DIPEA (9.46 mL, 73.8 mmol, 1.5 eq) in THF (30 mL) was added n-BuLi (1.9M, 31 mL, 59.0 mmol, 1.2 eq) at −78° C. After stirring for 30 min at −78° C., 4-bromo-2-ethyl-1-fluorobenzene solution (10.0 g, 49.2 mmol, 1.0 eq) in of THF (20.0 mL) was added. After stirring for 1 hr at −78° C., DMF (26.8 mL, 344 mmol, 7 eq) was added dropwise into the reaction mixture. After stirring at −78° C. for 30 min, the mixture was quenched with saturated aqueous NH₄Cl solution and extracted with ethyl acetate. The combined organic layer was washed with ice cold water, brine and dried over anhydrous Na2SO4, filtered and concentrated under reduced pressure. The residue was purified by gravity column chromatography (100-200 mesh silica gel) eluting with 2% ethyl acetate in hexane to afford 1.1 (7.9 g, 69%) as a yellow color liquid.

### Synthesis of 1.2. (General Procedure D):

To a stirred solution of tert-butyl (S)-3-aminopiperidine-1-carboxylate (5 g, 24.9 mmol, 1 eq) in DMF (50 mL) was added DIPEA (13 mL, 74.8 mmol, 3 eq) and 1H-pyrazole-1-carboximidamide hydrochloride (5.48 g, 37.4 mmol, 1.5 eq). Then the reaction mixture was stirred at 80° C. for 16 h. After completion of the reaction, the mixture was diluted with hexane that leads to the formation of a bi-layer. It was then stirred for 10 min at 25° C. Upper hexane layer was decanted. Same operation repeated 3 more times with the crude reaction mixture (i.e. with the lower layer), and upper hexane layer was decanted after each operation. After that, resultant crude reaction mixture was treated with a mixture of hexane and diethyl ether (70:30). Once again formation of bi-layer observed that was allowed to stir for another 10 min at 25° C. Upper layer decanted. Same operation repeated 3 more times with the crude reaction mixture until lower layer became sticky mass. This sticky mass dried under vacuum to form a sticky solid. It was then triturated with pentane several times and subsequently dried under vacuum to afford crude compound 1.2 (7 g) as white floppy solid. GC MS(−) (ES): m/z 243.9.

### Synthesis of 1.3. (General Procedure E):

To a stirred solution of 1.1 (2 g, 8.65 mmol, 1 eq) in DMA (14 mL) were added K₂CO₃ (3.6 g, 25.9 mmol, 3 eq) and 1.2 (3.14 g, 12.9 mmol, 1.5 eq) at 25° C. After stirring at 150° C. for 16, the mixture was diluted with water and extracted with ethyl acetate. The combined organic layer was washed with water, brine, dried over anhydrous Na2SO4, filtered and concentrated under reduced pressure. The residue was purified by combi-flash column chromatography (RedisepRf 40 g flash column) eluting with 22% ethyl acetate in hexane to afford 1.3 (700 mg, 19%) as a brown solid. MS(ES): m/z 434.9 [M+H]⁺, LCMS purity 93.54%.

### Synthesis of 1.4. (General Procedure F):

To a stirred solution of 1.3 (700 mg, 1.6 mmol, 1 eq) in dioxane (7 mL) were added KOAc (955 mg, 9.65 mmol, 6 eq) and 4,4,4′,4′,5,5,5′-heptamethyl-2,2′-bi(1,3,2-dioxaborolane) (888 mg, 3.70 mmol, 2.3 eq) at 25° C. The reaction mixture was degassed using nitrogen balloon for 10 min, treated with Pd(dppf)Cl₂ (235 mg, 0.32 mmol, 0.2 eq) and degassed again with nitrogen. After stirring at 90° C. for 16 h, the mixture was diluted with ethyl acetate. The organic layer was washed with water, brine and dried over anhydrous Na₂SO₄, filtered and concentrated. The residue was purified by combi-flash column chromatography (RedisepRf 40 g flash column) eluting with 28% ethyl acetate in hexane to afford 1.4 (900 mg) as a brown solid MS (ES): m/z 482.1 [M+H]⁺, LCMS purity 94.89%.

### Synthesis of 1.5. (General Procedure A):

To a stirred solution of 7-bromopyrrolo[2,1-f][1,2,4]triazin-4-amine (300 mg, 1.48 mmol, 1 eq) in DMF (2 mL) were added NaH (69.1 mg, 4.22 mmol, 3 eq) in portions at 0° C. followed by 2-fluorobenzenesulfonyl chloride (409 mg, 2.11 mmol, 1.5 eq). After stirring at 25° C. for 4 h, the reaction mixture was quenched with aqueous NH₄Cl and extracted with ethyl acetate. The combined organic layer was washed with washed with brine, dried over anhydrous Na₂SO₄, filtered and concentrated under reduced pressure. The residue was purified by combi-flash chromatography (RediSepRf 12 g flash column) eluting with 15% ethyl acetate in hexane to afford 1.5 (300 mg, 58%) as a white solid. MS(ES): m/z 370.8 [M+H]⁺, LCMS purity 94.67%.

### Synthesis of 1.6. (General Procedure B):

To a stirred solution of 1.5 (130 mg, 0.35 mmol, 1 eq) and 1.4 (202 mg, 0.42 mmol, 1.2 eq) in THF (4 mL) and H₂O (2 mL) was added K₃PO₄ (222 mg, 1.05 mmol, 3 eq). After degassing for 10 min under nitrogen atmosphere, XPhosPdG-3 (59.0 mg, 0.07 mmol, 0.2 eq) was added to the reaction mixture and degassed for another 5 min. After stirring at 80° C. for 16 h, the excess solvent was removed under reduced pressure, quenched with water and extracted with ethyl acetate. The combined organic layer was washed with brine and dried over anhydrous Na₂SO₄, filtered and concentrated under reduced pressure. The residue was purified by combi-flash chromatography (RediSepRf 12 g flash column) eluting with 80% ethyl acetate in hexane to afford 1.6 (140 mg). To 1.6 dissolved in dioxane (5 mL) was added silyl DMT-Pd-scavenger (200 mg). After refluxing for 4 h, the mixture was filtered through celite bed and the filtrate was concentrated under reduced pressure to afford 1.6 (130 mg, 57%) as a light yellow solid. MS(ES): m/z 647.2 [M+H]⁺, LCMS purity 96.33%.

### Synthesis of I-1. (General Procedure C):

To a stirred solution of 1.6 (130 mg, 0.20 mmol, 1 eq) in DCM (5 mL) was added 4M-HCl in dioxane (2 mL) at 0° C. After stirring at 25° C. for 2 h, the mixture was concentrated and the residue was purified by prep-HPLC using ACN/NH₄HCO₃ to afford I-1 (35.1 mg, 32%) as a solid. MS(ES): m/z 547.2 [M+H]⁺, LCMS purity 94.06%, HPLC purity 93.35%, 1H NMR (DMSO-d6, 400 MHz): 9.16 (s, 1H), 8.48 (bs, 2H), 8.18 (s, 1H), 7.91-7.87 (m, 1H), 7.69 (s, 1H), 7.47-7.42 (m, 2H), 7.25-7.21 (m, 1H), 7.17-7.13 (m, 1H), 7.01 (d, J=4.3 Hz, 1H), 6.70 (d, J=4.2 Hz, 1H), 4.22 (bs, 1H), 3.48-3.45 (m, 1H), 3.21-3.18 (m, 1H), 3.05 (q, J=7.4 Hz, 2H), 2.92-2.90 (m, 2H), 2.06-2.03 (m, 1H), 1.97-1.92 (m, 1H), 1.70-1.68 (m, 2H), 1.31 (t, J=7.4 Hz, 3H).

### Example 2: Synthesis of (S)—N-(7-(8-ethyl-2-(piperidin-3-ylamino)quinazolin-6-yl)pyrrolo[2,1-f][1,2,4]triazin-4-yl)-2-methylbenzenesulfonamide. I-2

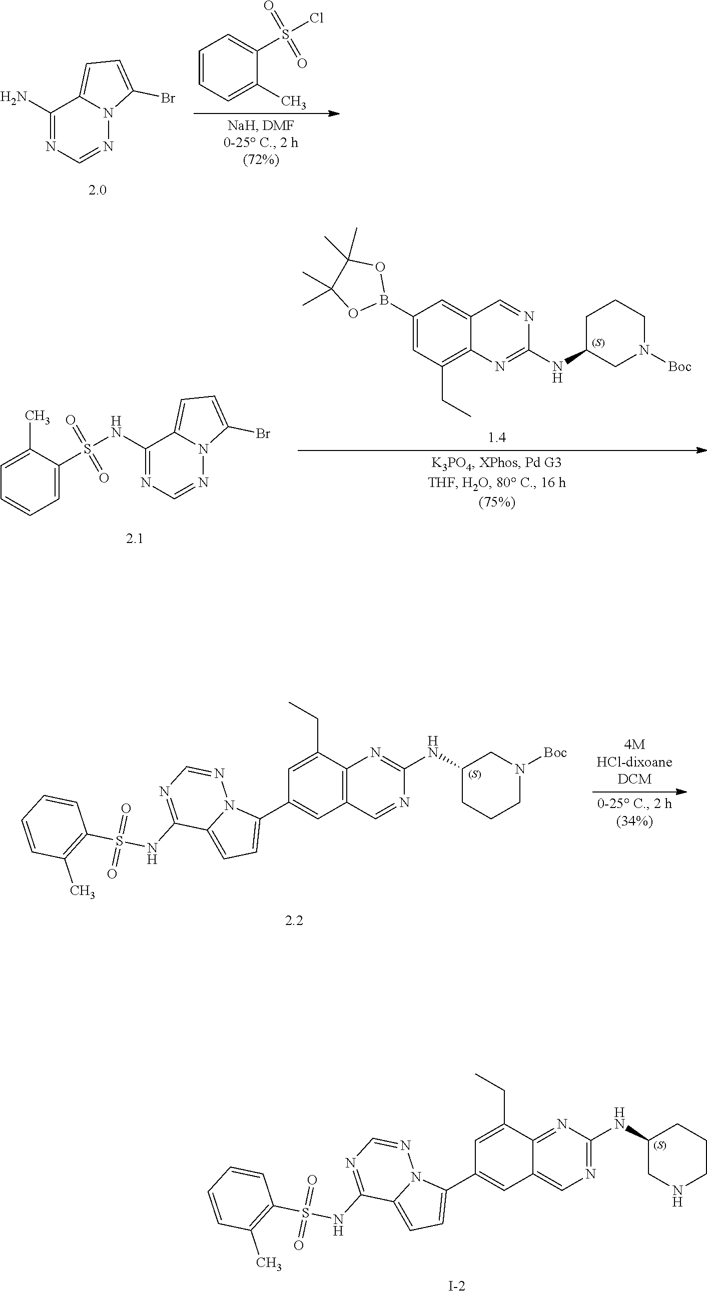

### Synthesis of 2.1.

2.1 was synthesized from 7-bromopyrrolo[2,1-f][1,2,4]triazin-4-amine (500 mg, 2.35 mmol, 1 eq) using general procedure A to afford 625 mg (Yield: 72%) as a solid. MS(ES): m/z 366.8 [M+H]⁺, LCMS purity 89.79%.

### Synthesis of 2.2.

2.2 was synthesized form 2.1 (70 mg, 0.18 mmol, 1 eq) and 1.4 (252 mg, 0.52 mmol, 3 eq) using general procedure B to afford 85.1 mg (Yield: 75%) as an off-white solid. MS(ES): m/z 643.2 [M+H]⁺, LCMS purity 94.71%.

### Synthesis of I-2.

I-2 was synthesized from 2.2 (85 mg, 0.13 mmol, 1 eq) using general procedure C (purified by prep-HPLC using ACN/NH₄HCO₃) to afford 25.1 mg (Yield: 34%) as a solid. MS(ES): m/z 543.2 [M+H]⁺, LCMS purity 96.76%, HPLC purity 98.20%, 1H NMR (DMSO-d6, 400 MHz): 9.15 (s, 1H), 8.47 (bs, 2H), 8.17 (s, 1H), 7.94 (d, J=7.4 Hz, 1H), 7.68 (s, 1H), 7.46 (bs, 1H), 7.29-7.23 (m, 2H), 7.17-7.15 (m, 1H), 6.98 (bs, 1H), 6.68 (bs, 1H), 4.21 (bs, 1H), 3.47-3.44 (m, 1H), 3.19-3.16 (m, 1H), 3.05-3.03 (m, 2H), 2.91-2.88 (m, 2H), 2.57 (s, 3H), 2.10-2.01 (m, 1H), 1.99-1.90 (m, 1H), 1.69-1.67 (m, 2H), 1.30 (t, J=7.1 Hz, 3H).

### Example 3: Synthesis of (S)—N-(7-(8-ethyl-2-(piperidin-3-ylamino)quinazolin-6-yl)pyrrolo[2,1-f][1,2,4]triazin-4-yl)cyclohexanesulfonamide. I-3

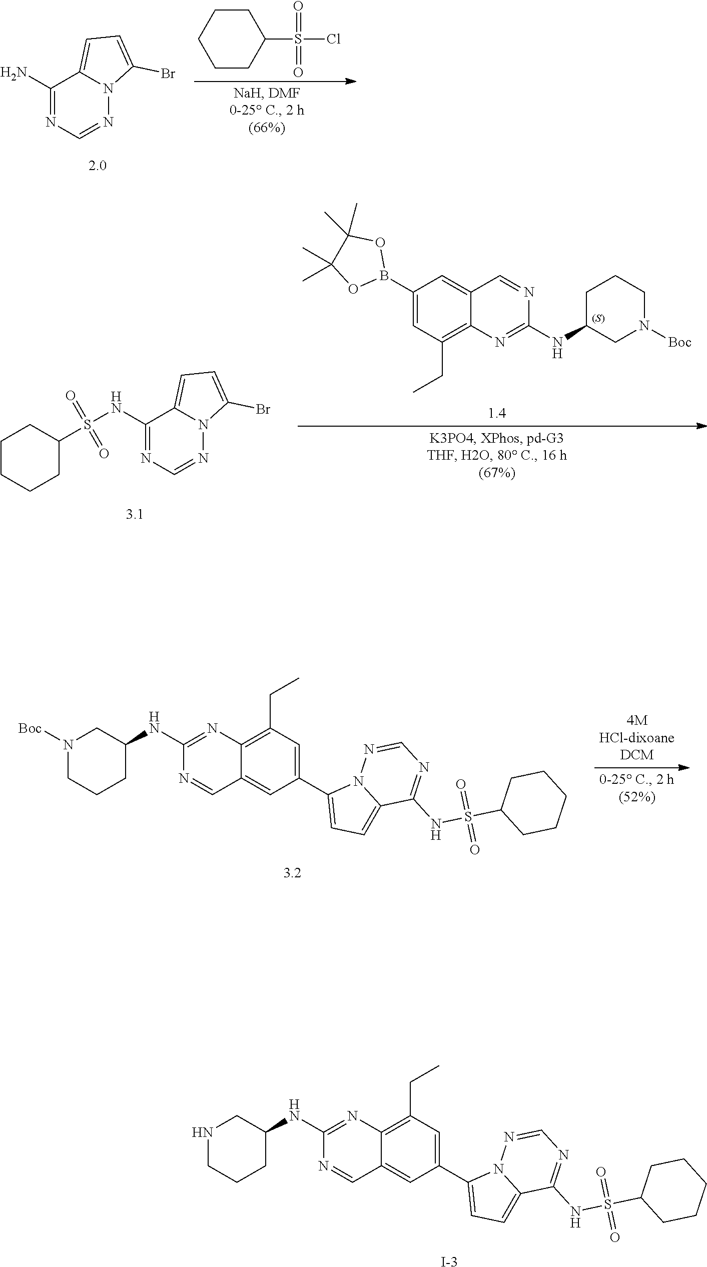

### Synthesis of 3.1

3.1 was synthesized from 7-bromopyrrolo[2,1-f][1,2,4]triazin-4-amine (100 mg, 0.47 mmol, 1 eq) and cyclohexanesulfonyl chloride (128 mg, 0.70 mmol, 1.5 eq) using general procedure A to obtain 80.0 mg (Yield: 66%) as an off-white solid. MS(ES): m/z 361.3 [M+H]⁺, LCMS purity 94.7%.

### Synthesis of 3.2.

3.2 was synthesized from 3.1 (80 mg, 0.22 mmol, 1 eq) and 1.4 (161 mg, 0.33 mmol, 1.5 eq) using general procedure B to obtain 100 mg (Yield: 67%) as an off-white solid. MS(ES): m/z 635.3 [M+H]⁺, LCMS purity 99%.

### Synthesis of I-3.

I-3 was synthesized from 3.2 (80 mg, 0.12 mmol, 1 eq) using general procedure C (purified by prep-HPLC using ACN/NH₄HCO₃) to obtain 34.8 mg (Yield: 52%) as a solid. MS(ES): m/z 534.2 [M+H]⁺, LCMS purity 97.91%, HPLC purity 97.21%, 1H NMR (DMSO-d6, 400 MHz): 9.18 (s, 1H), 8.70-8.58 (m, 1H), 8.50 (s, 1H), 8.19 (s, 1H), 7.88 (s, 1H), 7.49 (bs, 1H), 6.97 (d, J=4.3 Hz, 1H), 6.67 (d, J=3.7 Hz, 1H), 4.22 (bs, 1H), 3.55-3.41 (m, 3H), 3.20-3.17 (m, 1H), 3.03 (q, J=7.3 Hz, 2H), 2.94-2.87 (m, 2H), 2.00-1.91 (m, 4H), 1.77-1.60 (m, 5H), 1.38 (d, J=12.3 Hz, 2H), 1.31 (t, J=7.4 Hz, 3H), 1.23-1.18 (m, 3H).

### Example 4: Synthesis of 2-chloro-N-(7-(8-ethyl-2-(((1S,2S)-2-hydroxycyclohexyl)amino)quinazolin-6-yl)pyrrolo[2,1-f][1,2,4]triazin-4-yl)benzenesulfonamide. I-4

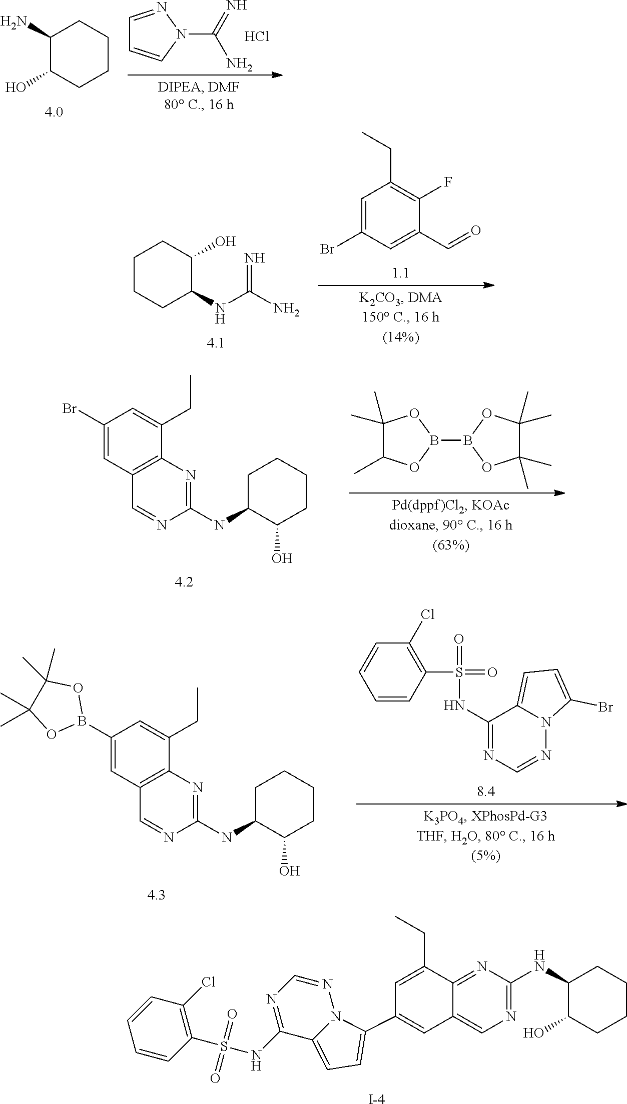

### Synthesis of 4.1.

4.1 was synthesized from tert(1S,2S)-2-aminocyclohexan-1-ol (500 mg, 4.34 mmol, 1 eq) using general procedure D to afford 500 mg as white solid. MS(ES): m/z 157.9 [M+H]⁺, LCMS purity 61.91%.

### Synthesis of 4.2.

4.2 was synthesized from 1.1 (500 mg, 2.30 mmol, 1 eq) and 4.1 (543 mg, 3.46 mmol, 1.5 eq) using general procedure E to obtain 110 mg (Yield: 14%) as a gum. MS(ES): m/z 350.0 [M+H]⁺, LCMS purity 98.98%.

### Synthesis of 4.3.

4.3 was synthesized from 4.2 (70 mg, 0.2 mmol, 1 eq) using general procedure F to obtain 50 mg (Yield: 63%) as a brown gum. MS (ES): m/z 397.6 [M+H]⁺, LCMS purity 77.38%.

### Synthesis of I-4.

I-4 was synthesized from 8.4 (62 mg, 0.16 mmol, 1 eq) prepared by General Procedure A and 4.3 (80 mg, 0.2 mmol, 1.3 eq) using general procedure B (purified by prep-HPLC using ACN/NH₄HCO₃) to obtain 5.5 mg (Yield: 5%) as a solid. MS(ES): m/z 578.4 [M+H]⁺, LCMS purity 96.68%, HPLC purity 96.13%, 1H NMR (DMSO-d6, 400 MHz): 12.04 (s, 1H), 9.12 (s, 1H), 8.32 (s, 1H), 8.16 (d, J=7.8 Hz, 1H), 8.09-8.08 (m, 2H), 7.65-7.59 (m, 2H), 7.57 (t, J=5.7 Hz, 1H), 7.27 (brs, 1H), 7.19-7.18 (m, 2H), 4.74 (brs, 1H), 3.70 (brs, 1H), 3.50 (brs, 1H), 3.07-2.95 (m, 2H), 2.14 (brs, 1H), 1.94-1.92 (m, 1H), 1.66 (brs, 1H), 1.30 (t, J=7.2 Hz, 3H), 1.27-1.23 (m, 4H).

### Example 5: Synthesis of (S)-4-(difluoromethoxy)-N-(7-(8-ethyl-2-(piperidin-3-ylamino)quinazolin-6-yl)pyrrolo[2,1-f][1,2,4]triazin-4-yl)benzenesulfonamide. I-5

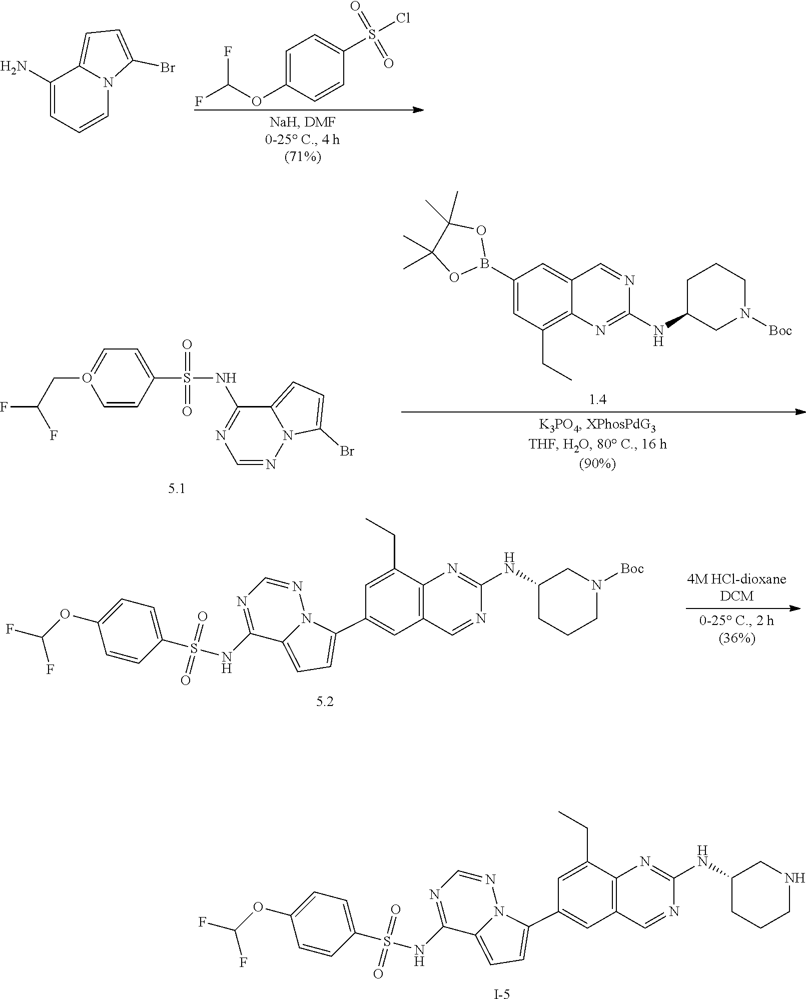

### Synthesis of 5.1.

5.1 was synthesized from 7-bromopyrrolo[2,1-f][1,2,4]triazin-4-amine (100 mg, 0.469 mmol, 1 eq) and 4-(difluoromethoxy)benzenesulfonyl chloride (123 mg, 0.704 mmol, 1.5 eq) using general procedure A to afford 140 mg (Yield: 71%) as a white solid. MS(ES): m/z 418.8 [M+H]⁺, LCMS purity 96.69%.

### Synthesis of 5.2.

5.2 was synthesized from 5.1 (80 mg, 0.191 mmol, 1 eq) and 1.4 (138 mg, 0.286 mmol, 1.5 eq) using general procedure B to afford 120 mg (Yield: 90%) as a light yellow solid. MS(ES): m/z 694.9 [M+H]⁺, LCMS purity 92.14%.

### Synthesis of I-5.

I-5 was synthesized from 5.2 (115 mg, 0.179 mmol, 1 eq) using general procedure C (purified by prep-HPLC using ACN/NH₄HCO₃) to afford 38 mg (Yield: 36%) as a solid. MS(ES): m/z 595.2 [M+H]⁺. LCMS purity 98.63%, HPLC purity 98.16%, 1H NMR (DMSO-d6, 400 MHz): 9.16 (s, 1H), 8.47 (s, 1H), 8.17-8.14 (m, 1H), 7.93 (d, J=8.6 Hz, 2H), 7.78 (s, 1H), 7.47-7.10 (m, 4H), 6.98 (d, J=4.3 Hz, 1H), 6.67 (d, J=4.3 Hz, 1H), 4.22 (bs, 1H), 3.48-3.45 (m, 1H), 3.21-3.18 (m, 1H), 3.04 (q, J=7.2 Hz, 2H), 2.92-2.89 (m, 2H), 2.04-2.02 (m, 1H), 1.93-1.91 (m, 1H), 1.74-1.65 (m, 2H), 1.30 (t, J=7.4 Hz, 3H).

### Example 6: Synthesis of (S)-2-chloro-N-(3-(8-ethyl-2-(piperidin-3-ylamino)quinazolin-6-yl)imidazo[1,2-a]pyrazin-8-yl)benzenesulfonamide. I-6

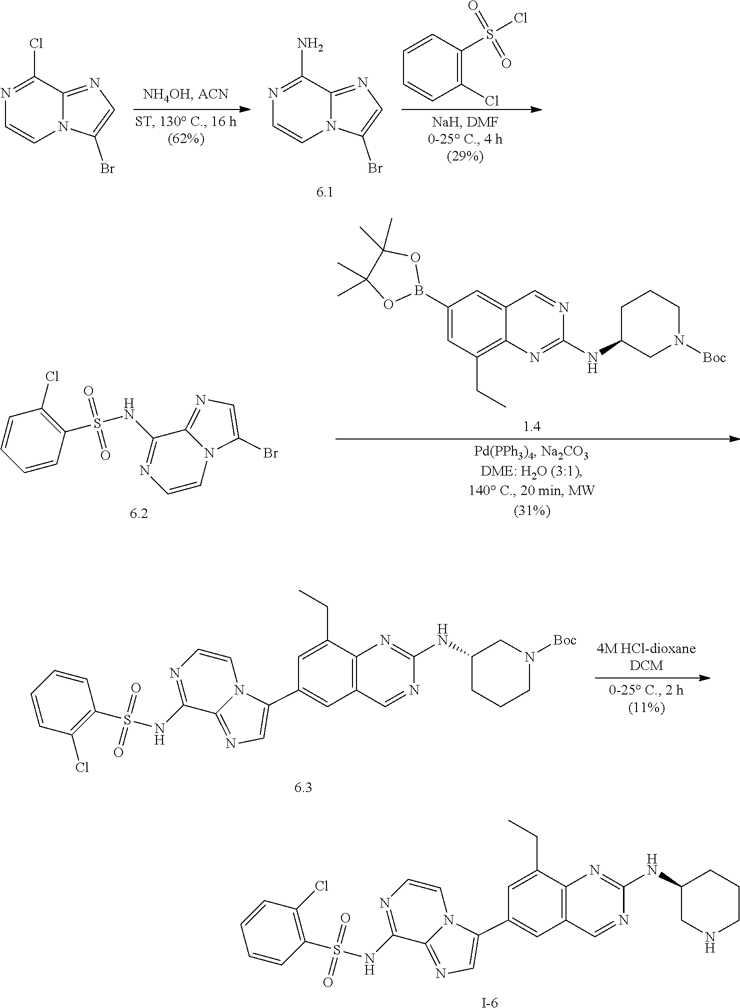

### Synthesis of 6.1.

To a stirred solution of 3-bromo-8-chloroimidazo[1,2-a]pyrazine (300 mg, 1.294 mmol, 1 eq) in CH₃CN (3 mL) was added NH₄OH (4 mL) in a sealed tube. After stirring at 130° C. for 16 h, the excess solvent was removed under reduced pressure. The material was filtered and washed with 5% MeOH in DCM. The filtrate was concentrated, and the residue washed with n-pentane to afford 6.1 (170 mg, 62%) as off-white solid. MS(ES): m/z 212.8 [M+H]⁺, LCMS purity 91.19%.

### Synthesis of 6.2.

6.2 was synthesized from 6.1 (170 mg, 0.79 mmol, 1 eq) and 2-chlorobenzenesulfonyl chloride (252 mg, 1.19 mmol, 1.5 eq) using general procedure A to afford 90 mg (Yield: 29%) as white solid. MS(ES): m/z 388.7 [M+H]⁺, LCMS purity 94.74%.

### Synthesis of 6.3.

To a stirred solution of 6.2 (140 mg, 0.36 mmol, 1 eq) in DME:H₂O (3:1) (4 mL) were added 1.4 (261 mg, 0.54 mmol, 1.5 eq) and Na₂CO₃ (76 mg, 0.72 mmol, 2 eq) in a microwave vial. After degassing for 10 min under nitrogen atmosphere, Pd(PPh₃)₄ (41 mg, 0.03 mol, 0.1 eq) was added and degassed for another 5 min. After the reaction mixture was stirred in microwave at 140° C. for 20 min, the mixture was diluted with water and extracted with ethyl acetate. The combined organic layer was washed with brine and concentrated under reduced pressure. The residue was purified by combi flash chromatography (RediSepRf 12 g flash column) eluting with 50% ethyl acetate in hexane to afford 6.3 (75.1 mg, 31%) as a light yellow solid. MS(ES): m/z 663.1 [M+H]⁺, LCMS purity 77.79%.

### Synthesis of I-6.

I-6 was synthesized from 6.3 (75 mg, 0.12 mmol, 1 eq) using general procedure C (purified by prep-HPLC using ACN/NH₄HCO₃) to afford 7.6 mg (Yield: 11%). MS(ES): m/z 561.1 [M−H]⁻, LCMS purity 95.21%, HPLC purity 90.45%. 1H NMR (DMSO-d6 at 100° C., 400 MHz): 9.13 (s, 1H), 8.17 (bs, 1H), 7.84 (s, 1H), 7.73 (s, 1H), 7.68-7.61 (m, 2H), 7.44-7.42 (m, 3H), 7.11 (bs, 1H), 6.87 (d, J=8.0 Hz, 1H), 4.08 (bs, 1H), 3.35-3.32 (m, 1H), 3.11-3.02 (m, 2H), 2.78-2.67 (m, 3H), 2.03-2.01 (m, 1H), 1.78-1.57 (m, 3H), 1.33 (t, J=8.0 Hz, 3H).

### Example 7: Synthesis of (S)—N-(7-(8-ethyl-2-(piperidin-3-ylamino)quinazolin-6-yl)pyrrolo[2,1-f][1,2,4]triazin-4-yl)-1-phenylmethanesulfonamide. I-7

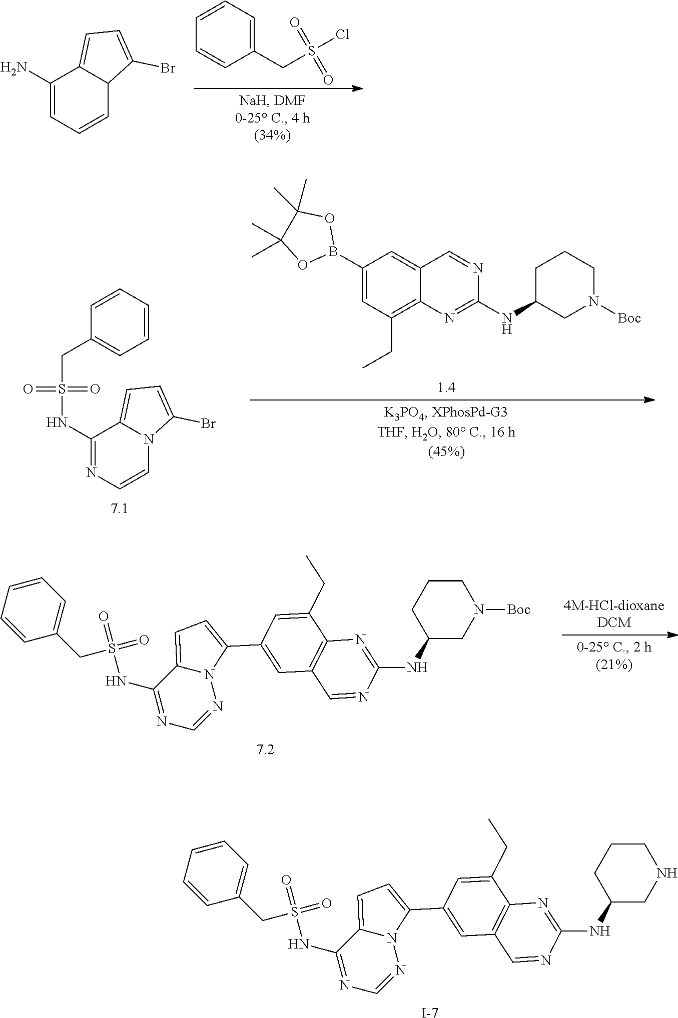

### Synthesis of 7.1.

7.1 was synthesized from 7-bromopyrrolo[2,1-f][1,2,4]triazin-4-amine (150 mg, 0.70 mmol, 1 eq) and phenylmethanesulfonyl chloride (186 mg, 1.06 mmol, 1.5 eq) to afford 90 mg (Yield: 34%) as white solid. MS(ES): m/z 366.9 [M+H]⁺, LCMS purity 85.18%.

### Synthesis of 7.2.

7.2 was synthesized from 7.1 (70 mg, 0.19 mmol, 1 eq) and 1.4 (137 mg, 0.28 mmol, 1.5 eq) using general procedure B to obtain 55 mg (Yield: 45%) as a light yellow solid. MS(ES): m/z 643.1 [M+H]⁺, LCMS purity 93.03%.

### Synthesis of I-7.

I-7 was synthesized from 7.2 (55 mg, 0.09 mmol, 1 eq) using general procedure C (purified by prep-HPLC using ACN/NH₄HCO₃) to afford 10 mg (Yield: 21%) as a solid. MS(ES): m/z 543.6 [M+H]⁺, LCMS purity 97.77%, HPLC purity 93.63%, 1H NMR (DMSO-d6, 400 MHz): 9.19 (s, 1H), 8.53 (s, 1H), 8.21-8.13 (m, 2H), 7.99 (s, 1H), 7.50 (bs, 1H), 7.28-7.24 (m, 5H), 6.98 (d, J=4.2 Hz, 1H), 6.62 (bs, 1H), 4.82 (s, 2H), 4.23 (bs, 1H), 3.49-3.46 (m, 1H), 3.21-3.18 (m, 1H), 3.08-3.06 (m, 2H), 2.93-2.91 (m, 2H), 2.09-1.99 (m, 1H), 1.98-1.90 (m, 1H), 1.71-1.69 (m, 2H), 1.32 (t, J=7.3 Hz, 3H).

### Example 8: Synthesis of (S)-2-chloro-N-(7-(8-ethyl-2-((piperidin-3-ylmethyl)amino)quinazolin-6-yl)pyrrolo[2,1-f][1,2,4]triazin-4-yl)benzenesulfonamide, I-8

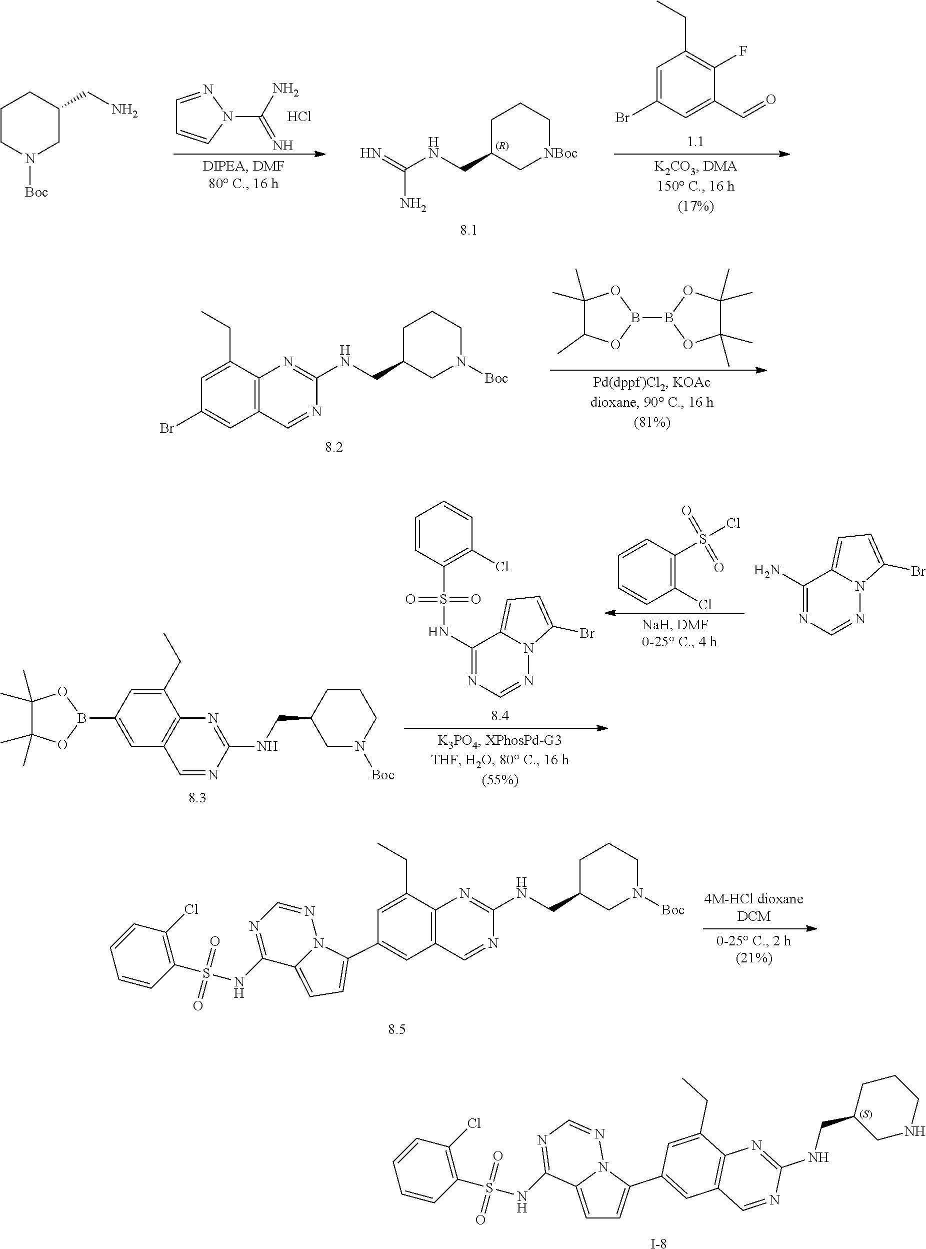

### Synthesis of 8.1.

8.1 was synthesized from tert-butyl (R)-3-(aminomethyl)piperidine-1-carboxylate (300 mg, 1.4 mmol, 1 eq) and 1H-pyrazole-1-carboximidamide hydrochloride (205 mg, 1.4 mmol, 1 eq) using general procedure D to afford 350 mg. MS(ES): m/z 2.150 [M+H]⁺, LCMS purity 79%.

### Synthesis of 8.2.

8.2 was synthesized from 8.1 (320 mg, 1.25 mmol, 1.7 eq) and 1.1 (170 mg, 0.74 mmol, 1 eq) using general procedure E to afford 55.1 mg (Yield: 17%) as light yellow solid. MS (ES): m/z 448.9 [M+H]⁺, LCMS purity 97.50%.

### Synthesis of 8.3.

8.3 was synthesized from 8.2 (50 mg, 0.11 mmol, 1 eq), using general procedure F to afford 45 mg (Yield: 81%) as off-white solid. MS(ES): m/z 497.0 [M+H]⁺, LCMS purity 89.99%.

### Synthesis of 8.4.

Using general procedure A, 8.4 was synthesized from 7-bromopyrrolo[2,1-f][1,2,4]triazin-4-amine (2.5 g, 11.7 mmol, 1 eq) and 2-chlorobenzenesulfonyl chloride (3.71 g, 17.6 mmol, 1.5 eq) to afford 1.2 g (Yield: 64%) as a light brown solid. MS(ES): m/z 377.6 [M+H]⁺, LCMS purity 74%).

### Synthesis of 8.5

8.5 was synthesized from 8.4 (25 mg, 0.06 mmol, 1 eq) and 8.3 (40 mg, 0.08 mmol, 1.3 eq) using general procedure B to afford 30 mg product (Yield: 55%) as yellow solid. MS(ES): m/z 677.1 [M+H]⁺, LCMS purity 65.09%.

### Synthesis of I-8.

I-8 was synthesized from 8.5 (45 mg, 0.07 mmol, 1 eq) using general procedure C (purified by prep-HPLC using ACN/ammonium bicarbonate) to obtain 8 mg (Yield: 21%) as a solid. MS(ES): m/z 577.2 [M+H]⁺, LCMS purity 97.78%, HPLC purity 97.17%, 1H NMR (DMSO-d6, 400 MHz): 9.11 (s, 1H), 8.45 (s, 1H), 8.15-8.14 (m, 1H), 8.07-8.06 (m, 1H), 7.67 (s, 1H), 7.56 (brs, 1H), 7.40-7.39 (m, 3H), 7.00 (d, J=4.32 Hz, 1H), 6.71 (d, J=4.1 Hz, 1H), 3.43 (m, 1H), 3.26-3.18 (m, 3H), 3.07-2.97 (m, 2H), 2.79 (t, J=11.5 Hz, 1H), 2.66 (t, J=12.0 Hz, 1H), 2.13 (brs, 1H), 1.82 (d, J=11.6 Hz, 2H), 1.60-1.56 (m, 1H), 1.31 (t, J=7.2 Hz, 3H), 1.26 (s, 1H).

### Example 9: Synthesis of (S)—N-(7-(8-ethyl-2-(piperidin-3-ylamino)quinazolin-6-yl)pyrrolo[2,1-f][1,2,4]triazin-4-yl)-3,3,3-trifluoropropane-1-sulfonamide. I-9

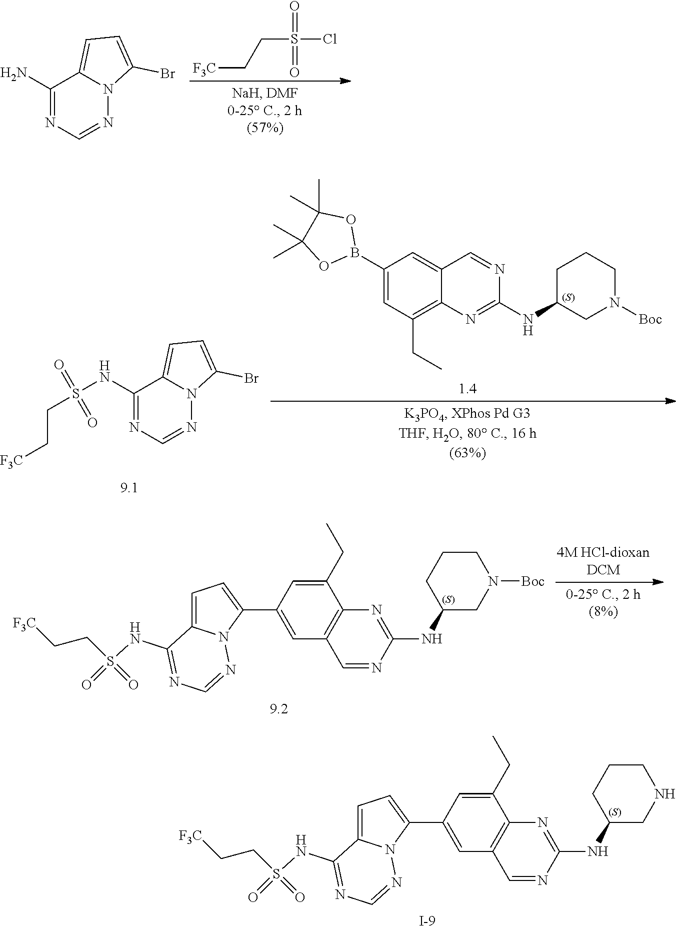

### Synthesis of 9.1.

9.1 was synthesized from 7-bromopyrrolo[2,1-f][1,2,4]triazin-4-amine (100 mg, 0.47 mmol, 1.0 eq) and 3,3,3-trifluoropropane-1-sulfonyl chloride (138 mg, 0.70 mmol, 1.5 eq) using general procedure A to obtain 100 mg (Yield: 57%) as yellow solid. MS(ES): m/z 372.9 [M+H]⁺, LCMS purity 96.23%.

### Synthesis of 9.2.

9.2 was synthesized from 9.1 (50 mg, 0.13 mmol, 1 eq), and 1.4 (97 mg, 0.2 mmol, 1.5 eq) using general procedure B to afford 55 mg (Yield: 63%) as brown solid. MS(ES): m/z 649.25 [M+H]⁺, LCMS purity 60.28%.

### Synthesis of I-9.

I-9 was synthesized from 9.2 (55 mg, 0.08 mmol, 1.0 eq) using general procedure C (purified by prep-HPLC using ACN/NH₄HCO₃) to afford 3.4 mg (Yield: 8%) as a solid. MS(ES): m/z 549.2 [M+H]⁺, LCMS purity 96.93%, HPLC purity 93.89%, 1H NMR (DMSO-d6, 400 MHz): 9.19 (s, 1H), 8.52 (s, 1H), 8.21-8.13 (m, 1H), 7.94 (s, 1H), 7.51 (bs, 1H), 7.01 (d, J=4.4 Hz, 1H), 6.66 (d, J=4.0 Hz, 1H), 4.25 (bs, 1H), 3.53-3.48 (m, 3H), 3.22-3.19 (m, 1H), 3.07-3.04 (m, 2H), 2.95-2.92 (m, 2H), 2.61-2.54 (m, 2H), 2.06-2.05 (m, 1H), 1.95-1.92 (m, 1H), 1.75-1.66 (m, 2H), 1.32 (t, J=7.48 Hz, 3H).

### Example 10: Synthesis of 2-chloro-N-(7-(8-ethyl-2-(((1S,2R)-2-hydroxycyclohexyl)amino)quinazolin-6-yl)pyrrolo[2,1-f][1,2,4]triazin-4-yl)benzenesulfonamide. I-10

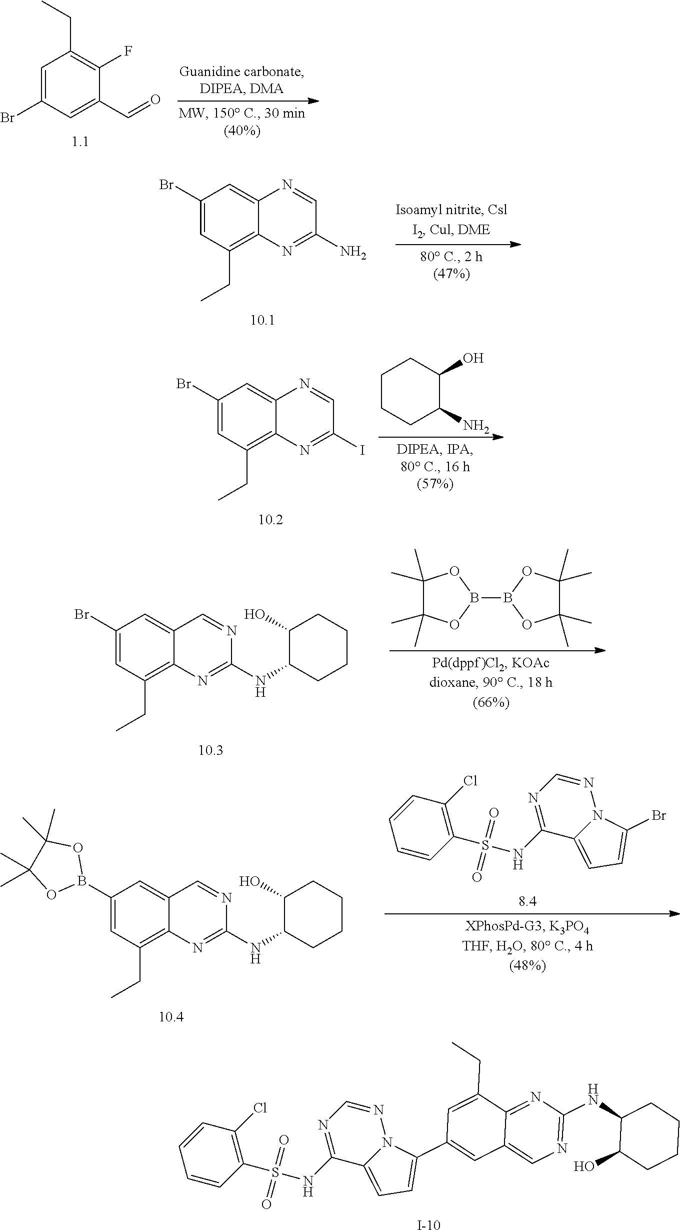

### Synthesis of 10.1.

To a stirred solution of guanidine·carbonate (389 mg, 2.16 mmol, 1 eq) in DMA (3 mL) were added DIPEA (0.34 mL, 2.16 mmol, 1 eq) and 1.1 (250 mg, 1.08 mmol, 0.5 eq) in a microwave vial. After heating in microwave at 150° C. for 30 minutes, the reaction mixture was quenched with ice cold water and extracted with ethyl acetate. The organic fraction was washed with brine, dried over anhydrous Na₂SO₄, filtered and concentrated under reduced pressure. The residue was purified by combi-flash column chromatography (RediSepRf 4 g flash column) using 15-17% ethyl acetate in hexane to afford 10.1 (110 mg, 40%) as a yellow solid. MS(ES): m/z 252.2 [M+H]⁺, LCMS purity 81%.

### Synthesis of 10.2.

To a stirred solution of 10.1 (150 mg, 0.59 mmol, 1 eq) in DME (3 mL) were added CsI (154 mg, 0.59 mmol, 1 eq), CuI (56.6 mg, 0.29 mmol, 0.5 eq), isoamyl nitrite (0.47 mL, 3.57 mmol, 6 eq) and Iodine (75.5 mg, 0.29 mmol, 0.5 eq). After stirring at 80° C. for 2 h, the reaction mixture was quenched with 25% aqueous NH₄OH solution followed by addition of aqueous saturated Na₂S₂O₃ solution, and extracted with ethyl acetate. The combined organic layer was washed with brine, dried over anhydrous Na₂SO₄, filtered and concentrated under reduced pressure. The residue was purified by combi-flash column chromatography (RediSepRf 4 g flash column) eluting with 5-7% ethyl acetate in hexane to afford 10.2 (100 mg, 47%) as brown solid. MS(ES): m/z 361.1 [M+H]⁺, LCMS purity 97.1%.

### Synthesis of 10.3. (General Procedure H):

To a stirred solution of 10.2 (200 mg, 0.55 mmol, 1 eq) in isopropanol (10 mL) were added (1R,2S)-2-aminocyclohexan-1-ol (250 mg, 1.65 mmol, 3 eq) and DIPEA (0.48 mL, 2.75 mmol, 5 eq). After stirring at 90° C. for 16 h, the volatiles were removed under vacuum. The residue was purified by combiflash (RediSepRf 4 g flash column) eluting with 5-15% ethyl acetate in hexane to afford 10.3 (100 mg, 57%) as off-white solid. MS(ES): m/z 349.7 [M+H]⁺, LCMS purity 93.1%.

### Synthesis of 10.4.

10.4 was synthesized from 10.3 (120 mg, 0.34 mmol, 1 eq) using general procedure F to afford 90 mg product (Yield: 66%) as yellow solid. MS(ES): m/z 397.8 [M+H]⁺, LCMS purity 90%.

### Synthesis of I-10.

I-10 was synthesized from 10.4 (90 mg, 0.23 mmol, 1 eq) and 8.4 (107 mg, 0.24 mmol, 1 eq) using general procedure B to obtain 20 mg (Yield: 52%) as a solid. MS(ES): m/z 577.8[M+H]⁺, LCMS purity 97%, HPLC purity 99.28%, 1H NMR (DMSO-d6, 400 MHz): 12.04 (bs, 1H), 9.13 (s, 1H), 8.34 (s, 1H), 8.16 (d, J=7.4 Hz, 1H), 8.09 (d, J=11.4 Hz, 2H), 7.67-7.62 (m, 2H), 7.59-7.54 (m, 1H), 7.27 (s, 1H), 7.19 (d, J=4.5 Hz, 1H), 6.80 (bs, 1H), 4.70 (s, 1H), 3.97 (bs, 2H), 2.98 (q, J=7.3 Hz, 2H), 1.78-1.48 (m, 6H), 1.34-1.28 (m, 5H).

### Example 11: Synthesis of (S)-2-chloro-N-(7-(8-ethyl-2-(pyrrolidin-3-ylamino)quinazolin-6-yl)pyrrolo[2,1-f][1,2,4]triazin-4-yl)benzenesulfonamide. I-11

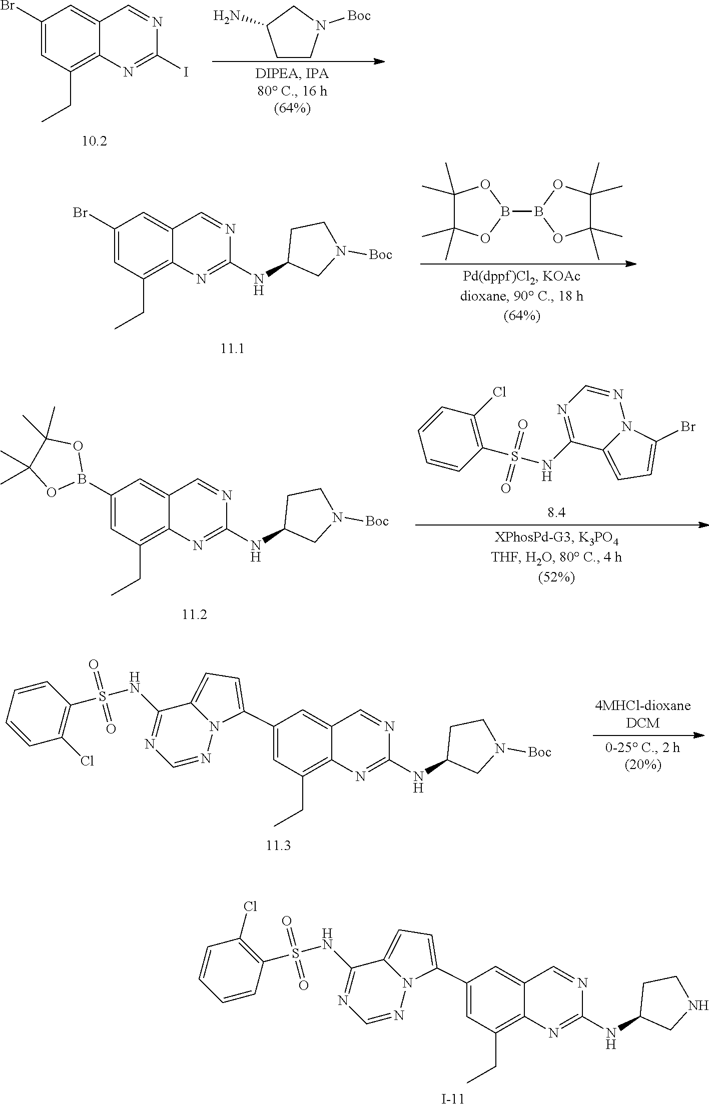

### Synthesis of 11.1.

11.1 was synthesized from 10.2 (200 mg, 0.55 mmol, 1 eq) and (S)-3-amino-1-(tert-butoxy(oxo)-λ⁵-methyl)pyrrolidine (307 mg, 1.65 mmol, 3 eq) using general procedure H to afford 150 mg (Yield: 64%) as yellow solid. MS(ES): m/z 420.7 [M+H]⁺, LCMS purity 90.2%.

### Synthesis of 11.2.

11.2 was synthesized from 11.1 (170 mg, 0.40 mmol, 1 eq) using general procedure F to obtain 120 mg (Yield: 64%) as brown solid. MS(ES): m/z 468.9 [M+H]⁺, LCMS purity 92.23%.

### Synthesis of 11.3.

11.3 was synthesized from 11.2 (160 mg, 0.34 mmol, 1 eq) and 8.4 (131 mg, 0.34 mmol, 1 eq) using general procedure B to obtain 120 mg (Yield: 54%) as yellow solid. MS(ES): m/z 649.2 [M+H]⁺, LCMS purity 72.13%.

### Synthesis of I-11.

I-11 was synthesized from 11.3 (100 mg, 0.15 mmol, 1 eq) and 8.4 (158 mg, 0.41 mmol, 1.5 eq) using general procedure B to obtain 30.1 mg (Yield: 54%) as a solid. MS(ES): m/z 549.2[M+H]⁺, LCMS purity 99%, HPLC purity 99.3%, 1H NMR (DMSO-d6, 400 MHz): 12.92 (bs, 1H), 9.26 (s, 1H), 8.25-8.21 (m, 2H), 8.01 (d, J=7.5 Hz, 1H), 7.42-7.31 (m, 4H), 6.83-6.80 (m, 2H), 4.18 (bs, 1H), 3.49-3.40 (m, 2H), 3.14-3.06 (m, 2H), 2.88-2.80 (m, 2H), 2.03 (bs, 1H), 1.90-1.88 (m, 1H), 1.66-1.67 (m, 2H), 1.33 (t, J=7.4 Hz, 3H).

### Example 12: Synthesis of (S)—N-(7-(2-(azepan-3-ylamino)-8-ethylquinazolin-6-yl)pyrrolo[2,1-f][1,2,4]triazin-4-yl)-2-chlorobenzenesulfonamide. I-12

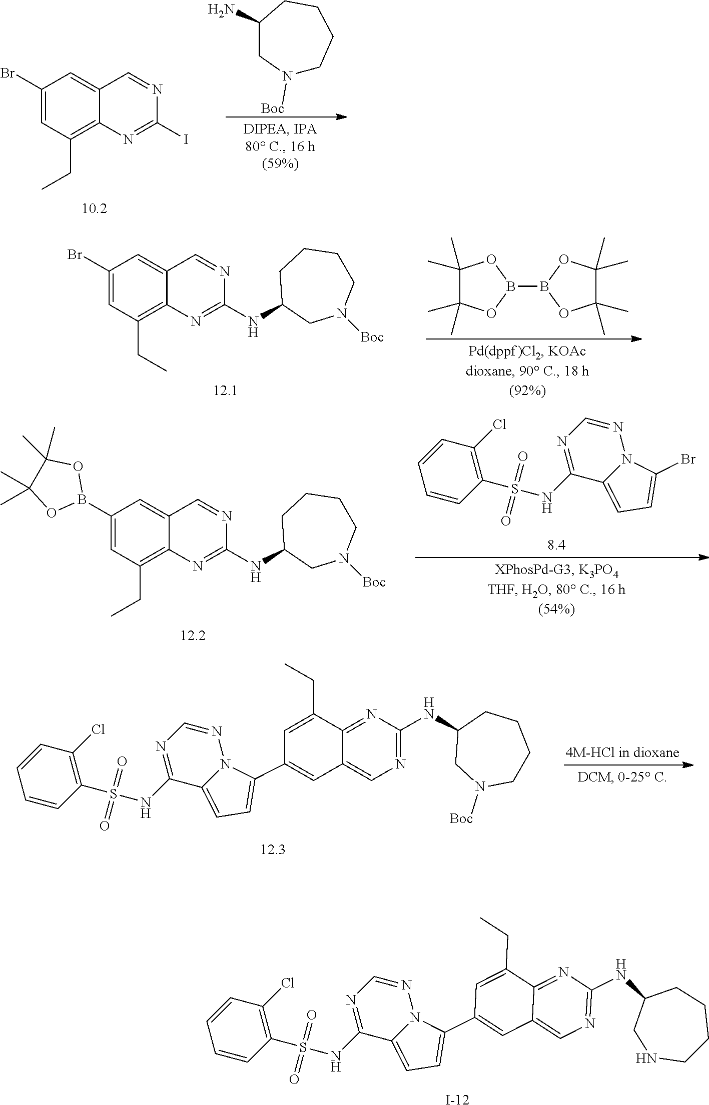

### Synthesis of 12.1.

12.1 was synthesized from 10.2 (200 mg, 0.55 mmol, 1 eq) and tert-butyl (S)-3-aminoazepane-1-carboxylate (250 mg, 1.65 mmol, 3 eq) using general procedure H to afford 100 mg (Yield: 58%) as yellow solid. MS(ES): m/z 349.7 [M+H]⁺, LCMS purity 96.1%.

### Synthesis of 12.2.

12.2 was synthesized from 12.1 (110 mg, 0.22 mmol, 1 eq) using general procedure F to afford 100 mg (Yield: 92%) as yellow solid. MS(ES): m/z 397.8 [M+H]⁺, LCMS purity 92.2%.

### Synthesis of 12.3.

12.3 was synthesized from 12.2 (90 mg, 0.23 mmol, 1 eq) and 8.4 (100 mg, 0.24 mmol, 1 eq) using general procedure B to obtain 80.1 mg (Yield: 54%) as white solid. MS(ES): m/z 677.8 [M+H]⁺, LCMS purity 95.6%.

### Synthesis of I-12.

I-12 was synthesized from 12.3 (110 mg, 0.22 mmol, 1 eq) using general procedure C to afford 2.3 mg (Yield: 2.0%) as yellow solid. MS(ES): m/z 576.9 [M+H]⁺, LCMS purity 88.98%, HPLC purity 92.93%, 1H NMR (CD3OD, 400 MHz): 9.03 (s, 1H), 8.33 (s, 1H), 8.19 (d, J=7.6 Hz, 1H), 8.10 (s, 1H), 7.73 (s, 1H), 7.55-7.35 (m, 3H), 7.08 (d, J=4.4 Hz, 1H), 6.96 (d, J=4.4 Hz, 1H), 4.37 (bs, 1H), 3.64-3.61 (m, 1H), 3.55-3.51 (m, 1H), 3.37-3.35 (m, 1H), 3.02 (q, J=7.2 Hz, 2H), 2.20-2.18 (m, 1H), 2.01-1.92 (m, 4H), 1.82-1.79 (m, 1H), 1.32 (t, J=7.4 Hz, 3H).

### Example 13: Synthesis of N-(7-(2-(((1S,2S)-2-aminocyclohexyl)amino)-8-ethylquinazolin-6-yl)pyrrolo[2,1-f][1,2,4]triazin-4-yl)-2-chlorobenzenesulfonamide. I-13

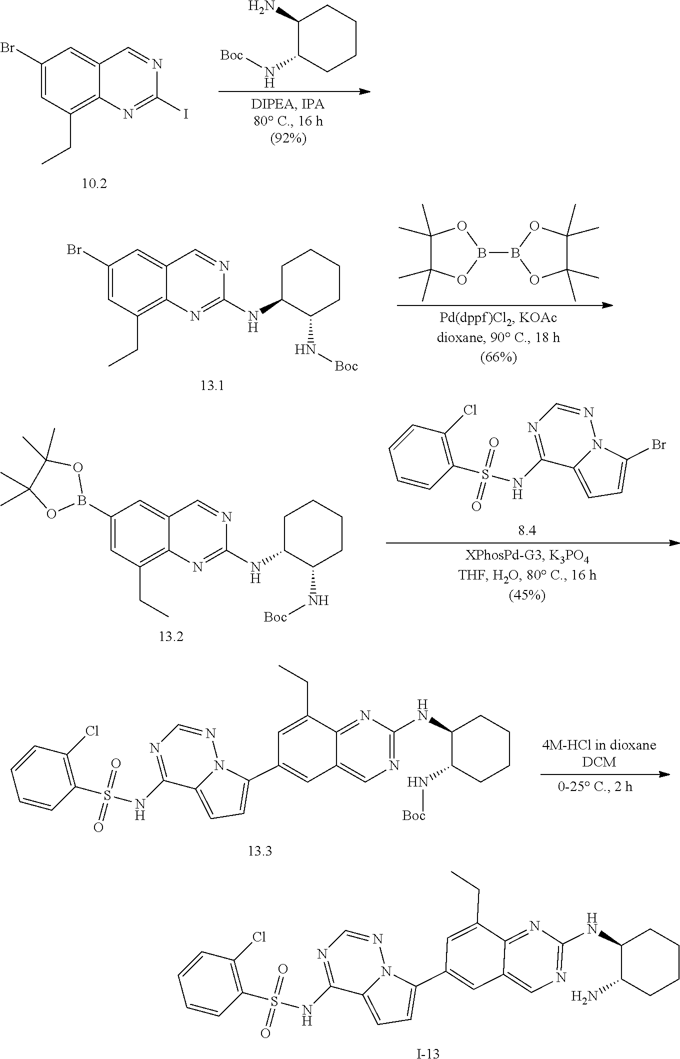

### Synthesis of 13.1.

13.1 was synthesized from 10.2 (150 mg, 0.41 mmol, 1 eq) and tert-butyl ((1S,2S)-2-aminocyclohexyl)carbamate (189 mg, 1.2 mmol, 3 eq) using general procedure H to obtain 140 mg (Yield: 92%) as yellow solid. MS(ES): m/z 393.7 [M+H]⁺, LCMS purity 97%.

### Synthesis of 13.2.

13.2 was synthesized from 13.1 (140 mg, 0.23 mmol, 1 eq) using general procedure F to obtain 100 mg (Yield: 75%) as white solid. MS(ES): m/z 497 [M+H]⁺, LCMS purity 90.1%.

### Synthesis of 13.3.

13.3 was synthesized from 13.2 (90 mg, 0.21 mmol, 1 eq) and 8.4 (107 mg, 0.24 mmol, 1 eq) using general procedure B to obtain 60.1 mg (Yield: 45%) as white solid. MS(ES): m/z 677.8[M+H]⁺, LCMS purity 97.2%.

### Synthesis of I-13.

I-13 was synthesized from 13.3 (50 mg, 0.19 mmol, 1 eq) using general procedure C to obtain 7.8 mg (Yield: 18%) as a solid. MS(ES): m/z 577.6 [M+H]⁺, LCMS purity 99.08%, HPLC purity 97.25%, 1H NMR (DMSO-d6, 400 MHz): 9.15 (bs, 1H), 8.47 (bs, 1H), 8.16 (s, 1H), 8.05-8.03 (m, 1H), 8.00-7.75 (m, 3H), 7.64 (s, 1H), 7.37 (d, J=2.8 Hz, 3H), 7.26 (bs, 1H), 6.98 (d, J=4.2 Hz, 1H), 6.67 (d, J=4.3 Hz, 1H), 3.93 (bs, 2H), 3.07-2.94 (m, 4H), 2.04-2.01 (m, 2H), 1.74-1.71 (m, 2H), 1.44-1.30 (m, 2H), 1.29 (t, J=7.0 Hz, 3H).

### Example 14: Synthesis of 2-chloro-N-(7-(8-ethyl-2-(((1S,3S)-3-hydroxycyclopentyl)amino)quinazolin-6-yl)pyrrolo[2,1-f][1,2,4]triazin-4-yl)benzenesulfonamide. I-14

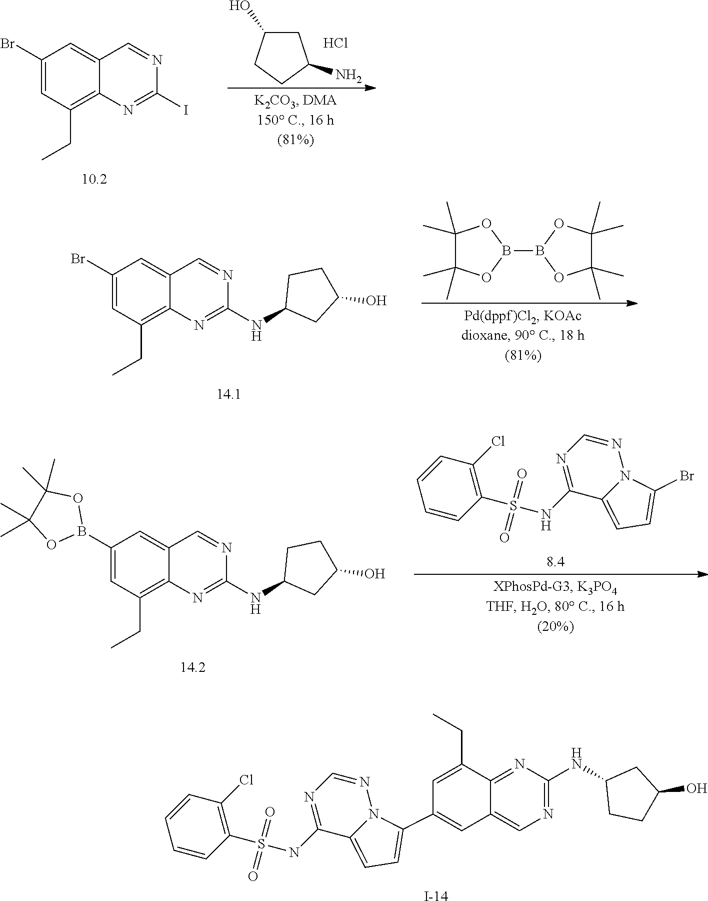

### Synthesis of 14.1.

14.1 was synthesized from 10.2 (100 mg, 0.28 mmol, 1 eq) and (1S,3S)-3-aminocyclopentan-1-ol hydrochloride (114 mg, 0.83 mmol, 3 eq) using general procedure H to obtain 75.2 mg (Yield: 81%) as off-white solid. MS(ES): m/z 336.1 [M+H]⁺, LCMS purity 92.94%.

### Synthesis of 14.2.

14.2 was synthesized from 14.1 (70 mg, 0.21 mmol, 1 eq) using general procedure F to afford 65.1 mg (Yield: 81%) as brown gum. MS(ES): m/z 383.8 [M+H]⁺, LCMS purity 80.37%.

### Synthesis of I-14.

I-14 was synthesized from 14.2 (65 mg, 0.17 mmol, 1 eq) and 8.4 (53 mg, 0.14 mmol, 0.8 eq) using general procedure C to obtain 16.1 mg (Yield: 20%) as a solid. MS(ES): m/z 564.3 [M+H]⁺, LCMS purity 99.14%, HPLC purity 98.68%. 1H NMR (DMSO-d6, 400 MHz): 12.04 (s, 1H), 9.10 (s, 1H), 8.32 (s, 1H), 8.16 (d, J=7.8 Hz, 1H), 8.09 (d, J=8.5, 2H), 7.69-7.62 (m, 2H), 7.58-7.55 (m, 2H), 7.26 (brs, 1H), 7.18 (d, J=4.6, 1H,), 4.51 (brs, 2H), 4.25 (brs, 1H), 3.06-3.00 (m, 2H), 2.18 (m, 1H), 1.98-1.91 (m, 2H), 1.80-1.75 (m, 1H), 1.57-1.51 (m, 2H), 1.31 (t, J=7.4, 3H).

### Example 15: Synthesis of (S)—N-(7-(8-ethyl-2-(piperidin-3-ylamino)quinazolin-6-yl)pyrrolo[2,1-f][1,2,4]triazin-4-yl)cyclopentanesulfonamide. I-15

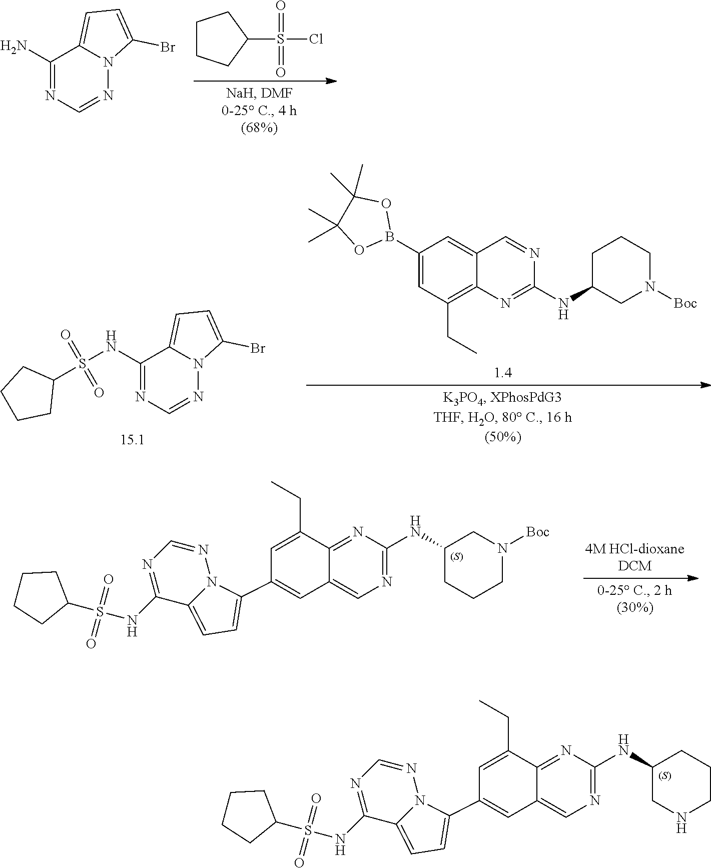

### Synthesis of 15.1.

15.1 was synthesized from 7-bromopyrrolo[2,1-f][1,2,4]triazin-4-amine (100 mg, 0.47 mmol, 1 eq) and cyclopentanesulfonyl chloride (119 mg, 0.70 mmol, 1.5 eq) using general procedure A to afford to afford 15.1 (110 mg, 68%) as a yellowish gummy. MS (ES): m/z 345 [M+H]⁺, LCMS purity 85.57%.

### Synthesis of 15.2.

15.2 was synthesized from 15.1 (100 mg, 0.28 mmol, 1 eq) and 1.4 (168 mg, 0.34 mmol, 1.2 eq) using general procedure B to obtain 90 mg (Yield: 50%) as yellowish gum. MS (ES): m/z 621.2 [M+H]⁺, LCMS purity 96.01%.

### Synthesis of I-15.

I-15 was synthesized from 15.2 (90 mg, 0.14 mmol, 1 eq) using general procedure C to afford 23.5 mg (Yield: 30%) as a solid. MS (ES): m/z 519.2 [M−H]⁻, LCMS purity 99.33%, HPLC purity 98.54%, 1H NMR (DMSO-d6, 400 MHz): 9.17 (s, 1H), 8.50 (s, 1H), 8.18 (d, J=11.4 Hz, 1H), 7.87 (s, 1H), 7.47 (d, J=4.9, 1H), 6.97 (d, J=4.3 Hz, 1H), 6.65 (d, J=4.2 Hz, 1H), 4.21-4.15 (m, 2H), 3.47-3.44 (m, 1H), 3.20-3.16 (m, 1H), 3.05 (q, J=7.28 Hz, 2H), 2.90-2.88 (m, 2H), 2.05-2.02 (m, 1H), 1.93-1.87 (m, 3H), 1.78-1.65 (m, 6H), 1.52-1.49 (m, 2H), 1.31 (t, J=7.4 Hz, 3H).

### Example 16: Synthesis of (S)-2-chloro-N-(7-(8-ethyl-2-(piperidin-3-ylamino)quinazolin-6-yl)-5-fluoropyrrolo[2,1-f][1,2,4]triazin-4-yl)benzenesulfonamide. I-16

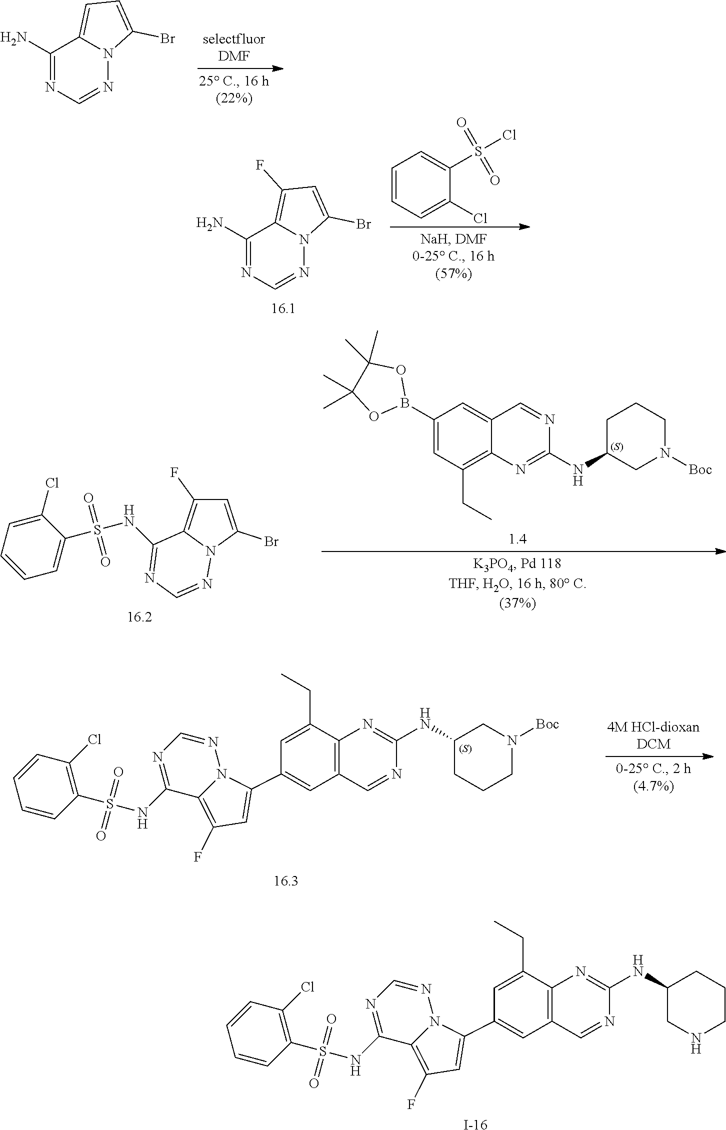

### Synthesis of 16.1.

To a stirred solution of 7-bromopyrrolo[2,1-f][1,2,4]triazin-4-amine (100 mg, 0.47 mmol, 1 eq) in DMF (2 mL) was added selectfluor (166 mg, 0.47 mmol, 1 eq) at 25° C. After stirring at 25° C. for 16 h, the reaction mixture was diluted with water and extracted with ethyl acetate. The combined organic layer was washed with aqueous NaHCO₃ solution, brine, dried over anhydrous Na₂SO₄, filtered and concentrated under reduced pressure. The residue was purified by combi-flash column chromatography (RediSepRf 12 g flash column) eluting with 30% ethyl acetate in hexane to afford 16.1 (24 mg, 22%) as white solid. MS(ES): m/z 231.0 [M+H]⁺, LCMS purity 45.4%.

### Synthesis of 16.2.

16.2 was synthesized from 16.1 (200 mg, 0.86 mmol, 1 eq) and 2-chlorobenzenesulfonyl chloride (274 mg, 1.29 mmol, 1.5 eq) using general procedure A to afford 200 mg (Yield: 57%) as light brown solid. MS(ES): m/z 405.2 [M+H]⁺, LCMS purity 42.8%.

### Synthesis of 16.3.

To a stirred solution of 16.2 (80 mg, 0.19 mmol, 1 eq) and 1.4 (114 mg, 0.23 mmol, 1.2 eq) in dioxane (8 mL) and H₂O (2 mL) (4:1) was added potassium carbonate (125 mg, 0.59 mmol, 3.0 eq). After degassing for 10 min under argon atmosphere, [PdCl2(dtbpf)] (12.8 mg, 0.02 mmol, 0.1 eq) was added to the reaction mixture and degassed for another 10 min. After stirring at 90° C. for 16 h, the reaction mixture was filtered through celite bed and the filtrate was diluted with ethyl acetate. The combined organic layer was washed with water, dried over anhydrous Na₂SO₄, filtered and concentrated. The residue was purified by combi-flash column chromatography (RediSepRf 12 g flash column) eluting with 40% ethyl acetate in hexane to afford 16.3 (50.1 mg, 37%) as light brown solid. MS(ES): m/z 681.4 [M+H]⁺, LCMS purity 64.9%.

### Synthesis of I-16.

I-16 was synthesized from 16.3 (50 mg, 0.07 mmol, 1.0 eq) using general procedure C to obtain 2.02 mg (Yield: 4.7%) as a solid. MS(ES): m/z 581.1 [M+H]⁺, LCMS purity 97.15%, HPLC purity 97.3%, 1H NMR (DMSO-d6, 400 MHz): 9.15 (bs, 1H), 8.45 (bs, 1H), 8.12 (d, J=3.2 Hz, 1H), 8.07-8.07 (m, 1H), 7.53 (s, 1H), 7.41-7.38 (m, 3H), 6.89 (s, 1H), 4.22 (bs, 1H), 3.47 (d, J=11.9 Hz, 1H), 3.19 (d, J=12.6 Hz, 1H), 3.00 (q, J=7.3 Hz, 2H), 2.93-2.90 (m, 2H), 2.07-2.03 (m, 1H), 1.92-1.90 (m, 1H), 1.74-1.64 (m, 2H), 1.30 (t, J=7.4 Hz, 3H).

### Example 17: Synthesis of (R)-2-chloro-N-(7-(8-ethyl-2-((piperidin-3-ylmethyl)amino)quinazolin-6-yl)pyrrolo[2,1-f][1,2,4]triazin-4-yl)benzenesulfonamide. I-17

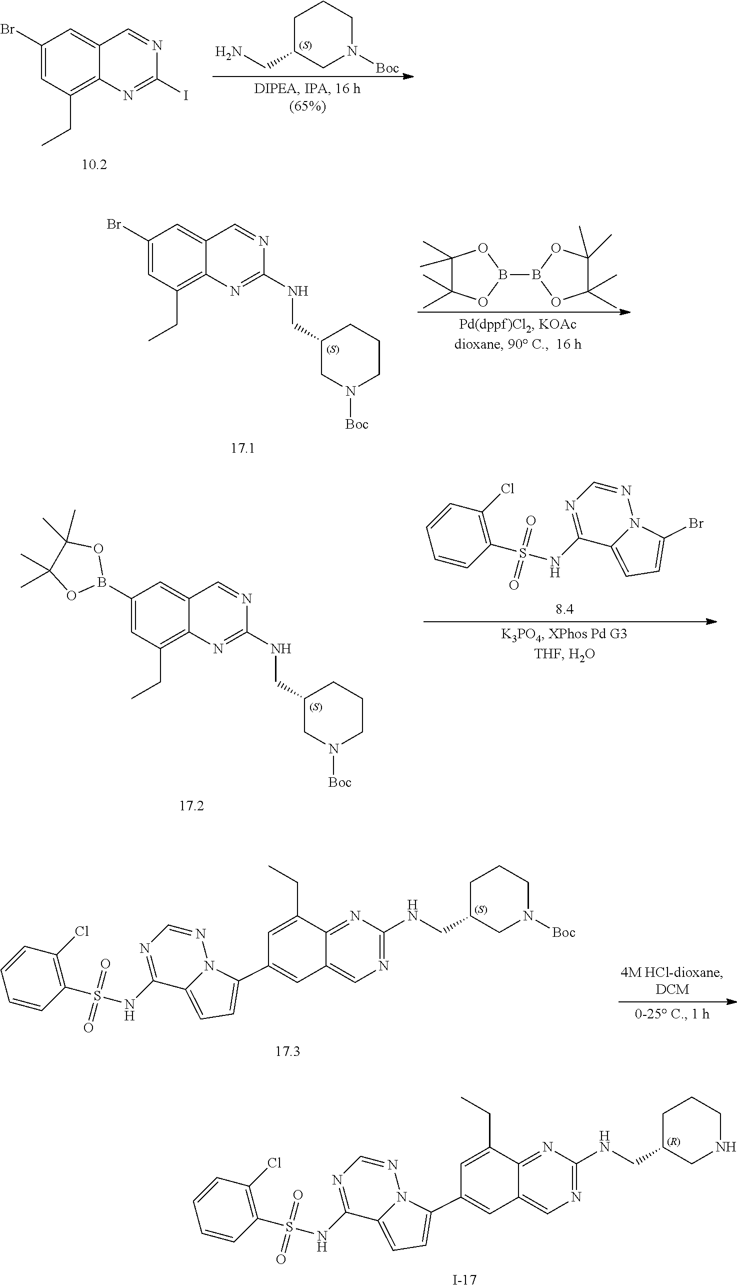

### Synthesis of 17.1.

17.1 was synthesized from 10.2 (200 mg, 0.55 mmol, 1 eq) and tert-butyl (S)-3-(aminomethyl)piperidine-1-carboxylate (236 mg, 1.10 mmol, 2 eq) using general procedure H to afford 17.1 (160 mg, 65%) as a yellowish gum MS (ES): m/z 448.8 [M+H]⁺, LCMS purity 99.8%.

### Synthesis of 17.2.

17.2 was synthesized from 17.1 (160 mg, 0.35 mmol, 1 eq) using general procedure F to afford 17.2 (136 mg, 76%) as yellowish gum. MS (ES): m/z 497.2 [M+H]⁺, LCMS purity 92.11%.

### Synthesis of 17.3.

17.3 was synthesized from 17.2 (130 mg, 0.26 mmol, 1 eq) and 8.4 (122 mg, 0.31 mmol, 1.2 eq) using general procedure B to afford 80 mg (Yield: 45%) as a yellowish gum. MS (ES): m/z 677.1 [M+H]⁺, LCMS purity 99.7%.

### Synthesis of I-17.

I-17 was synthesized 17.3 (76 mg, 0.11 mmol, 1 eq) using general procedure C to afford 9.7 mg (Yield: 15%) as a solid. MS(ES): m/z 577.2 [M+H]⁺, LCMS purity 97.2%, HPLC purity 97.03%, 1H NMR (DMSO-d6, 400 MHz): 9.10 (s, 1H), 8.45 (s, 1H), 8.16 (bs, 1H), 8.08-8.05 (m, 1H), 7.65 (s, 1H), 7.55 (bs, 1H), 7.40-7.39 (m, 3H), 6.99 (d, J=4.3 Hz, 1H), 6.68 (d, J=4.3 Hz, 1H), 3.46-3.40 (m, 2H), 3.19 (d, J=12.7 Hz, 2H), 3.07-2.99 (m, 2H), 2.79-2.75 (m, 1H), 2.68-2.62 (m, 1H), 2.19-2.15 (m, 1H), 1.81 (d, J=11.3 Hz, 2H), 1.61-1.46 (m, 1H), 1.31 (t, J=7.2 Hz, 3H), 1.26-1.23 (m, 1H).

### Example 18: Synthesis of (S)-2-chloro-N-(7-(8-ethyl-2-((piperidin-2-ylmethyl)amino)quinazolin-6-yl)pyrrolo[2,1-f][1,2,4]triazin-4-yl)benzenesulfonamide. I-18

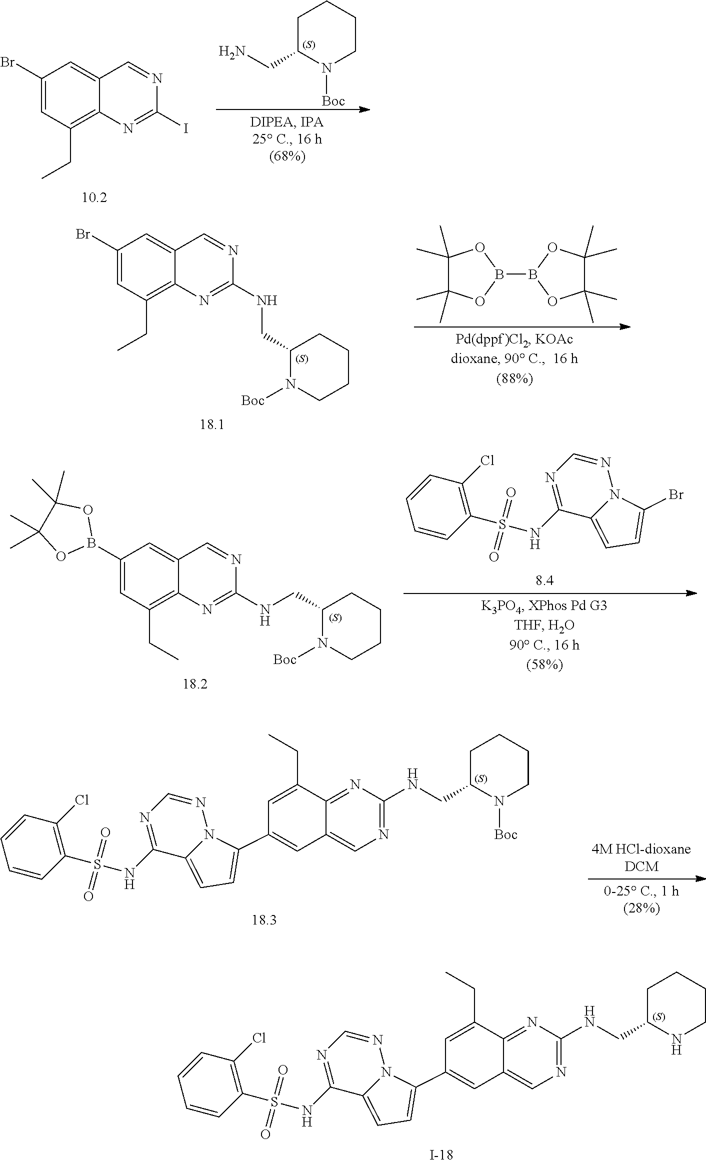

### Synthesis of 18.1.

18.1 was synthesized from 10.2 (200 mg, 0.55 mmol, 1 eq) and tert-butyl (S)-2-(aminomethyl)piperidine-1-carboxylate (236 mg, 1.10 mmol, 2 eq) using general procedure H to afford 170 mg (Yield: 68%) as an yellowish gum MS (ES): m/z 450.8 [M+H]⁺, LCMS purity 98.74%.

### Synthesis of 18.2.

18.2 was synthesized from 18.1 (170 mg, 0.37 mmol, 1 eq) using general procedure F to afford 157 mg (Yield: 88%) as a yellowish gum. MS (ES): m/z 497.0 [M+H]⁺, LCMS purity 93.62%.

### Synthesis of 18.3.

18.3 was synthesized from 18.2 (150 mg, 0.30 mmol, 1 eq) and 8.4 (140 mg, 0.36 mmol, 1.2 eq) using general procedure B to afford 120 mg (Yield: 58%) as a yellowish gum. MS (ES): m/z 676.8 [M+H]⁺, LCMS purity 100%.

### Synthesis of I-18.

I-18 was synthesized from 18.3 (110 mg, 0.16 mmol, 1 eq) using general procedure C to afford 26.8 mg (Yield: 28.6%) as a solid. MS(ES): m/z 577.2 [M+H]⁺, LCMS purity 99.1%, HPLC purity 98.47%, 1H NMR (DMSO-d6, 400 MHz): 9.17 (s, 1H), 8.50 (s, 1H), 8.19 (bs, 1H), 8.08-8.06 (m, 1H), 7.66 (s, 1H), 7.53 (bs, 1H), 7.40-7.39 (m, 3H), 7.00 (d, J=4.3 Hz, 1H), 6.70 (d, J=4.3 Hz, 1H), 3.64-3.56 (m, 2H), 3.48-3.45 (m, 1H), 3.25 (d, J=13.1 Hz, 2H), 3.04 (d, J=7.1 Hz, 2H), 2.84 (t, J=9.9 Hz, 1H), 1.89 (d, J=6.9 Hz, 1H), 1.80-1.70 (m, 2H), 1.59-1.46 (m, 3H), 1.30 (t, J=7.4 Hz, 3H).

### Example 19: Synthesis of (S)-2-chloro-N-(3-(8-ethyl-2-(piperidin-3-ylamino)quinazolin-6-yl)-1H-indazol-7-yl)benzenesulfonamide. I-19

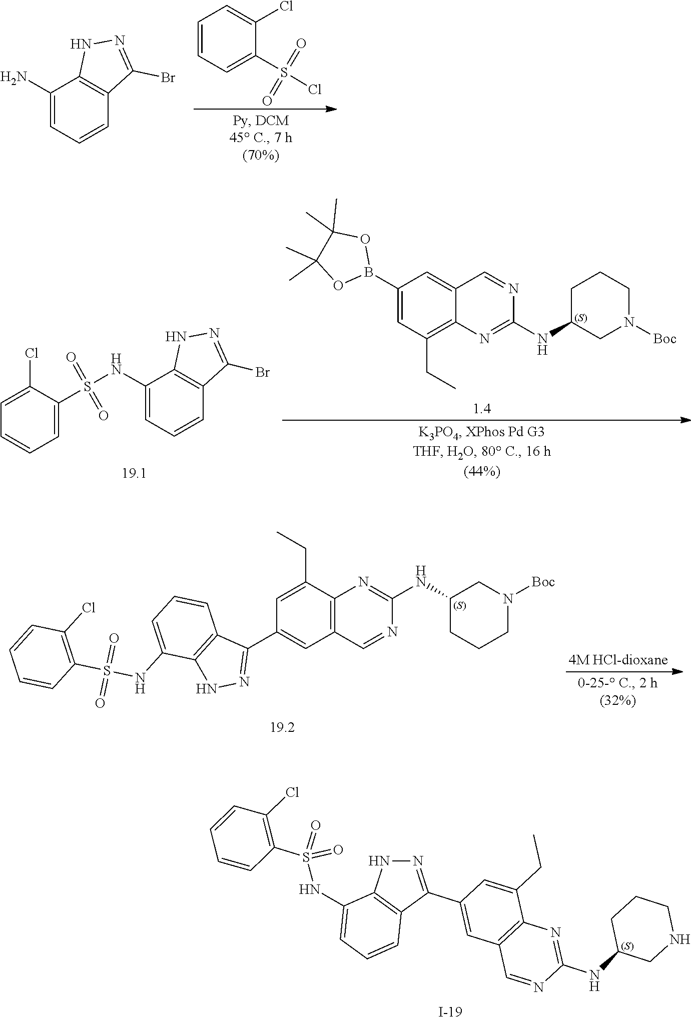

### Synthesis of 19.1.

To a stirred solution of 2-chlorobenzenesulfonyl chloride (29.7 mg, 0.94 mmol, 1 eq) in DCM (2 mL) and pyridine (5 mL, 1.08 mmol, 12 eqv) was added 3-bromo-1H-indazol-7-amine (20 mg, 0.09 mmol, 1.5 eq). After stirring at 115° C. for 7 h, the reaction mixture was quenched with citric acid and extracted with DCM. The combined organic layer was washed with brine, dried over anhydrous Na₂SO₄, filtered and concentrated under reduced pressure. The residue was purified by combi-flash column chromatography (RediSepRf 4 g flash column) eluting with 12-15% ethyl acetate in hexane to afford 19.1 (25.1 mg, Yield: 70%). MS(ES): m/z 388.8 [M+H]⁺, LCMS purity 59.2%.

### Synthesis of 19.2.

19.2 was synthesized from 19.1 (200 mg, 0.51 mmol, 1 eq) and 1.4 (300 mg, 0.62 mmol, 1.2 eq) using general procedure B to afford 19.2. 180 mg (Yield: 44%). MS(ES): m/z 662.1 [M+H]⁺, LCMS purity 97.1%).

### Synthesis of I-19.

I-19 was synthesized from 19.2 (150 mg, 0.22 mmol, 1 eq) using general procedure C to afford 40 mg (Yield: 32%) as a solid. MS(ES): m/z 562.2 [M+H]⁺, LCMS purity 99%, HPLC purity 99.3%, 1H NMR (DMSO-d6, 400 MHz): 12.92 (bs, 1H), 9.26 (s, 1H), 8.25-8.21 (m, 2H), 8.01 (d, J=7.5 Hz, 1H), 7.42-7.31 (m, 4H), 6.83-6.80 (m, 2H), 4.18 (bs, 1H), 3.49-3.40 (m, 2H), 3.14-3.06 (m, 2H), 2.88-2.80 (m, 2H), 2.03 (bs, 1H), 1.90-1.88 (m, 1H), 1.66-1.67 (m, 2H), 1.33 (t, J=7.4 Hz, 3H).

### Example 20: Synthesis of (R)-1-((7-(8-ethyl-2-(((S)-piperidin-3-yl)amino)quinazolin-6-yl)pyrrolo[2,1-f][1,2,4]triazin-4-yl)amino)butan-2-ol. I-20

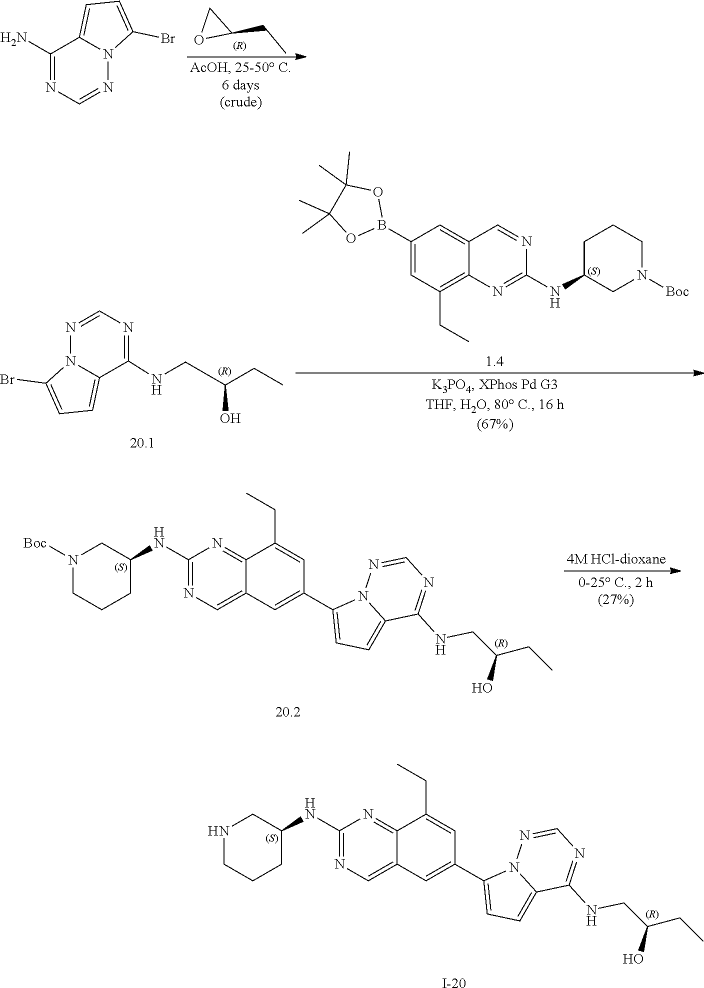

### Synthesis of 20.1.

To a suspension of 7-bromopyrrolo[2,1-f][1,2,4]triazin-4-amine (1.0 g, 4.69 mmol, 1 eq) in AcOH (33 mL) was added (R)-2-ethyloxirane (0.44 mL, 5.16 mmoL, 1.1 eq) at 25° C. under nitrogen. After stirring at 25° C. for 16 h and then at 50° C. for 1 h, additional (R)-2-ethyloxirane (0.44 mL, 5.16 mmoL, 1.1 eq) was added with stirring at 25° C. for 72 h. The reaction was continued for another 3 days heating at 50° C. for 1 h each day. Additional (R)-2-ethyloxirane (0.44 mL, 5.16 mmoL, 1.1 eq) was added and then stirred at 25° C. overnight. The reaction mixture was evaporated under reduced pressure, neutralized with aq NaHCO₃ solution, and extracted with EtOAc. The combined organic solution was dried (anhydrous Na₂SO₄), concentrated. The residue was purified by combiflash column chromatography (RediSepRf 12 g flash column) (eluting with 40-45% ethyl acetate-hexane) to afford a mixture of 20.1 and 7-bromopyrrolo[2,1-f][1,2,4]triazin-4-amine (180 mg, ˜1:1) as a solid which was used for the next step without further purification. MS(ES): m/z 284.7 [M+H]⁺, LCMS purity 46.2%.

### Synthesis of 20.2.

20.2 was synthesized from 20.1 and 7-bromopyrrolo[2,1-f][1,2,4]triazin-4-amine (180 mg, ˜1:1) (180 mg, 0.632 mmol, 1 eq) and 8.4 (458 mg, 0.95 mmol, 1.5 eq) using general procedure B to afford 120 mg (Yield: 67%) as brown solid. MS(ES): m/z 560.1 [M+H]⁺. LCMS purity 81.7%.

### Synthesis of I-20:

I-20 was synthesized from 20.2 (120 mg, 0.21 mmol, 1 eq) using general procedure C to afford 27 mg (Yield: 27%) as a solid. MS(ES): m/z 461.3 [M+H]⁺, LCMS purity 97.46%, HPLC purity 96.96%, 1H NMR (DMSO-d6, 400 MHz): 9.07 (s, 1H), 8.16 (s, 1H), 7.97 (bs, 2H), 7.75 (s, 1H), 7.19 (bs, 1H), 7.04 (d, J=3.9 Hz, 1H), 6.76 (d, J=4.32 Hz, 1H), 5.10 (bs, 1H), 4.18-4.14 (m, 1H), 3.89 (bs, 1H), 3.72 (bs, 1H), 3.53-3.48 (m, 1H), 3.14-3.05 (m, 1H), 2.97 (q, J=7.4 Hz, 2H), 2.79-2.77 (m, 1H), 1.91 (bs, 1H), 1.64-1.61 (m, 1H), 1.51-1.31 (m, 4H), 1.27 (t, J=7.4 Hz, 3H), 0.90 (t, J=7.3 Hz. 3H).

### Example 21: Synthesis of (S)-1-(2,3-difluorophenyl)-N-(7-(8-ethyl-2-(piperidin-3-ylamino)quinazolin-6-yl)pyrrolo[2,1-f][1,2,4]triazin-4-yl)methanesulfonamide. I-21

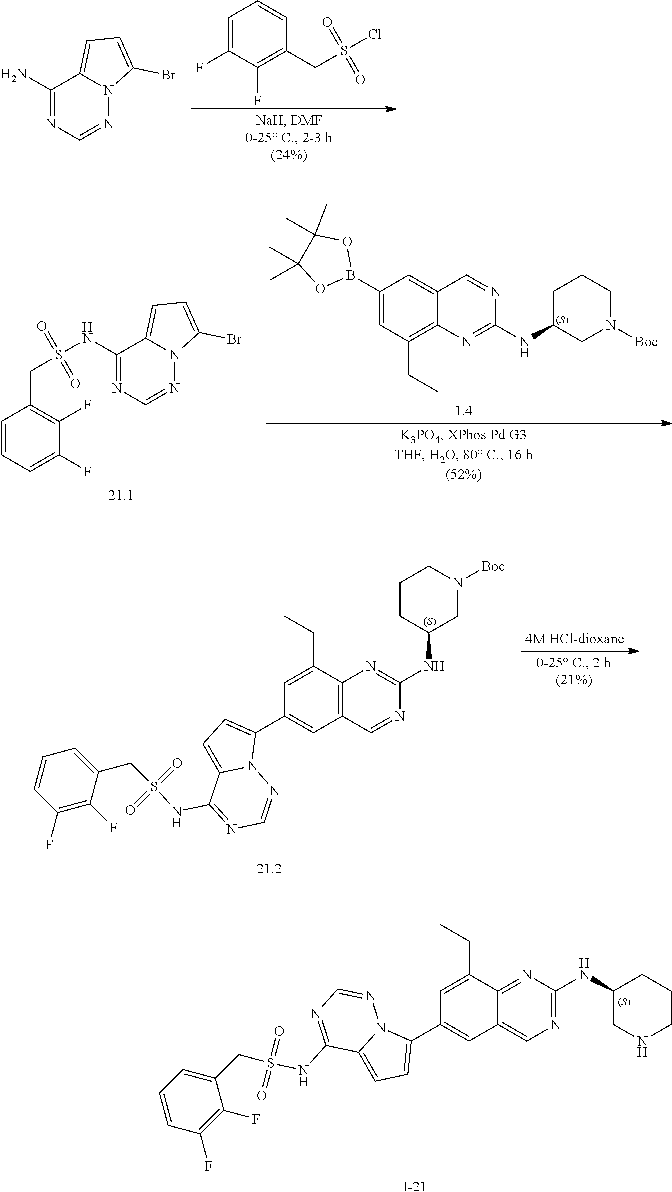

### Synthesis of 21.1.

21.1 was synthesized from 7-bromopyrrolo[2,1-f][1,2,4]triazin-4-amine (100 mg, 0.47 mmol, 1.0 eq) and (2,3-difluorophenyl)methanesulfonyl chloride (158 mg, 0.70 mmol, 1.5 eq) using general procedure A to obtain 45 mg (Yield: 24%) as yellow solid. MS(ES): m/z 402.9 [M+H]⁺, LCMS purity 90.72%.

### Synthesis of 21.2.

21.2 was synthesized from 21.1 (80 mg, 0.2 mmol, 1 eq) and 1.4 (143 mg, 0.3 mmol, 1.5 eq) using general procedure B to obtain 70 mg (Yield: 52%) as yellow solid. MS(ES): m/z 679.2 [M+H]⁺, LCMS purity 90.06%.

### Synthesis of I-21.

I-21 was synthesized from 21.2 (65 mg, 0.10 mmol, 1.0 eq) using general procedure C to afford 12 mg (Yield: 21%) as a solid. MS(ES): m/z 579.6 [M+H]⁺, LCMS purity 95.93%, HPLC purity 95.22%, 1H NMR (DMSO-d6, 400 MHz): 9.19 (s, 1H), 8.54 (s, 1H), 8.21-8.13 (m, 2H), 8.00 (s, 1H), 7.50 (bs, 1H), 7.32-7.29 (m, 1H), 7.19-7.10 (m, 2H), 7.01 (d, J=4.3 Hz, 1H), 6.63 (bs, 1H), 4.71 (s, 2H), 4.25 (bs, 1H), 3.50-3.47 (m, 1H), 3.22-3.19 (m, 1H), 3.1 (q, J=7.4 Hz, 2H), 2.93-2.91 (m, 2H), 2.06-2.04 (m, 1H), 1.99-1.93 (m, 1H), 1.75-1.66 (m, 2H), 1.32 (t, J=7.4 Hz, 3H).

### Example 22: Synthesis of (S)-2-chloro-N-(7-(8-ethyl-2-(piperidin-3-ylamino)quinazolin-6-yl)pyrrolo[2,1-f][1,2,4]triazin-4-yl)benzenesulfonamide. I-22

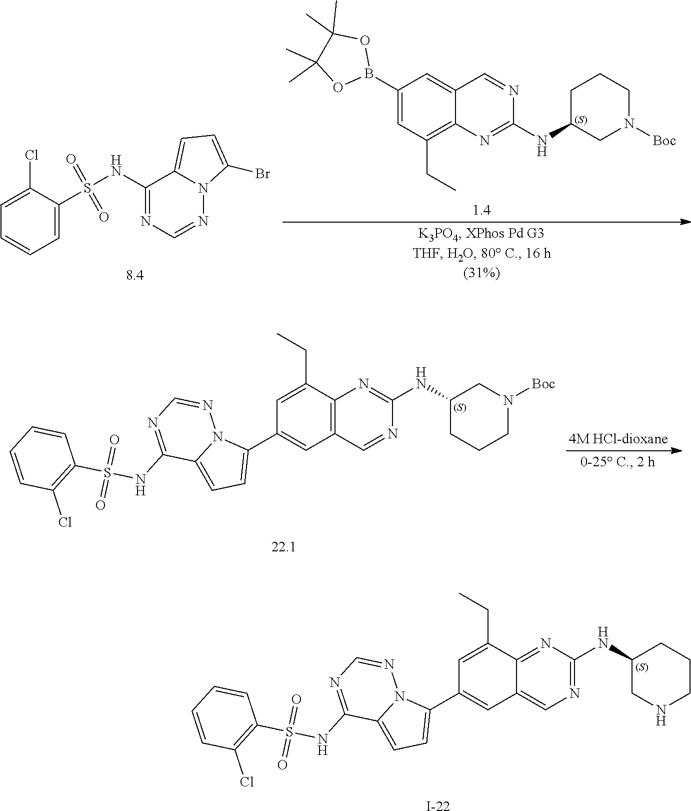

### Synthesis of 22.1.

22.1 was synthesized from 8.4 (1.5 g, 3.87 mmol, 1 eq) and 1.4 (2.05 g, 4.2 mmol, 1.1 eq) using general procedure B to afford 1.1 g (Yield: 43%). MS(ES): m/z 663.2 [M+H]⁺, LCMS purity 88.97%.

### Synthesis of I-22.

I-22 was synthesized from 22.1 (130 mg, 0.19 mmol) using general procedure C to obtain 42.13 mg (Yield: 40%) as a solid. MS(ES): m/z 563.2 [M+H]⁺, LCMS purity 99%, HPLC purity 98.4%, 1H NMR (DMSO-d6, 400 MHz): 9.16 (s, 1H), 8.47 (bs, 2H), 8.17 (s, 1H), 8.08-8.05 (m, 1H), 7.66 (s, 1H), 7.49-7.47 (m, 1H), 7.39 (d, J=2.7 Hz, 3H), 7.01 (d, J=4.4 Hz, 1H), 6.70 (d, J=4.1 Hz, 1H), 4.22 (bs, 1H), 3.48-3.45 (m, 1H), 3.21-3.16 (m, 1H), 3.07-3.02 (m, 2H), 2.92-2.90 (m, 2H), 2.05-2.02 (m, 1H), 1.91-1.90 (m, 1H), 1.74-1.68 (m, 2H), 1.30 (t, d=7.4 Hz, 3H).

### Example 23: Synthesis of (S)-2-chloro-N-(7-(8-ethyl-2-(piperidin-3-ylamino)quinazolin-6-yl)thieno[3,2-d]pyrimidin-4-yl)benzenesulfonamide. I-23

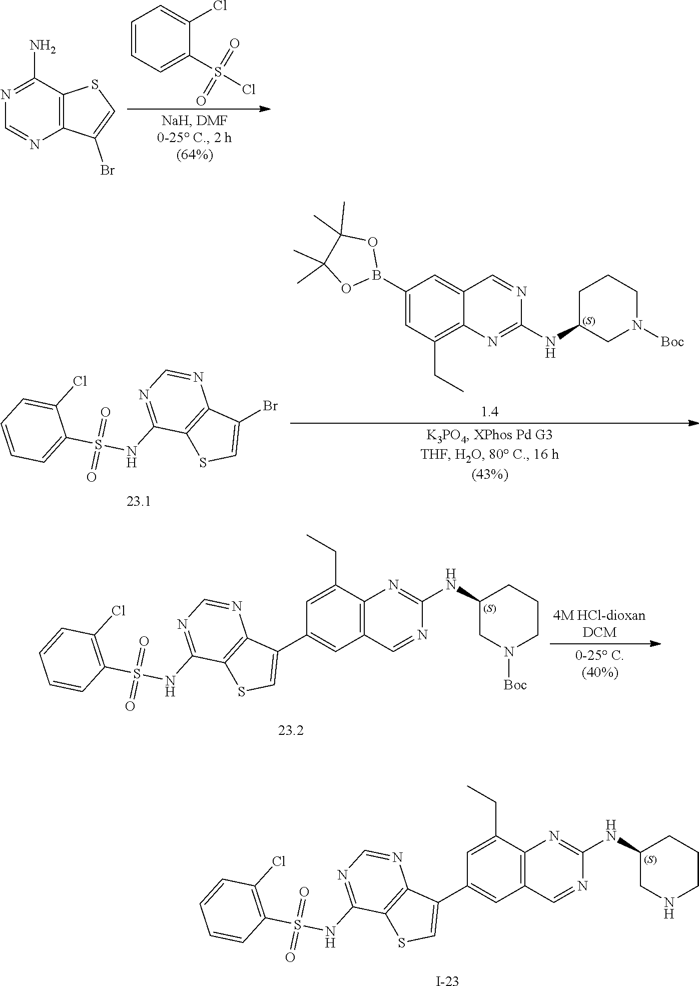

### Synthesis of 23.1.

23.1 was synthesized from 7-bromothieno[3,2-d]pyrimidin-4-amine (200 mg, 0.87 mmol, 1 eq) and 2-chlorobenzenesulfonyl chloride (276.54 mg, 1.31 mmol, 1.5 eq) using general procedure A to obtain 190 mg 23.1 (Yield: 54%) as light brown solid. MS(ES): m/z 405.7[M+H]⁺, LCMS purity 91.35%.

### Synthesis of 23.2.

23.2 was synthesized from 23.1 (100 mg, 0.24 mmol, 1 eq) and 1.4 (178 mg, 0.37 mmol, 1.5 eq) using general procedure B to obtain 110 mg (Yield: 65%) as an off-white solid. MS(ES): m/z 668.1[M+H]⁺, LCMS purity 94.63%.

### Synthesis of I-23.

I-23 was synthesized from 23.2 (100 mg, 0.15 mmol, 1 eq) using general procedure C to obtain 9.07 mg (Yield: 11%) as a solid. MS(ES): m/z 580.12 [M+H]⁺, LCMS purity 98.22%, HPLC purity 97.90%, 1H NMR (DMSO-d6 at 20° C., 400 MHz): 9.18 (s, 1H), 8.54 (s, 1H), 8.22 (d, J=12 Hz, 1H), 8.09 (bs, 1H), 7.49 (bs, 1H), 7.37 (bs, 3H), 4.22 (bs, 1H), 3.48-3.45 (m, 1H), 3.22-3.20 (m, 2H), 3.05 (d, J=6.6 Hz, 2H), 2.92-2.90 (m, 2H), 2.06-2.04 (m, 1H), 1.93-1.91 (m, 1H), 1.74-1.68 (m, 2H), 1.30 (t, d=7.4 Hz, 3H).

### Example 24: Synthesis of 3-cyclopropyl-1-(7-{8-ethyl-2-[(3R)-piperidin-3-ylamino]quinazolin-6-yl}pyrrolo[2,1-f][1,2,4]triazin-4-yl)urea. I-24

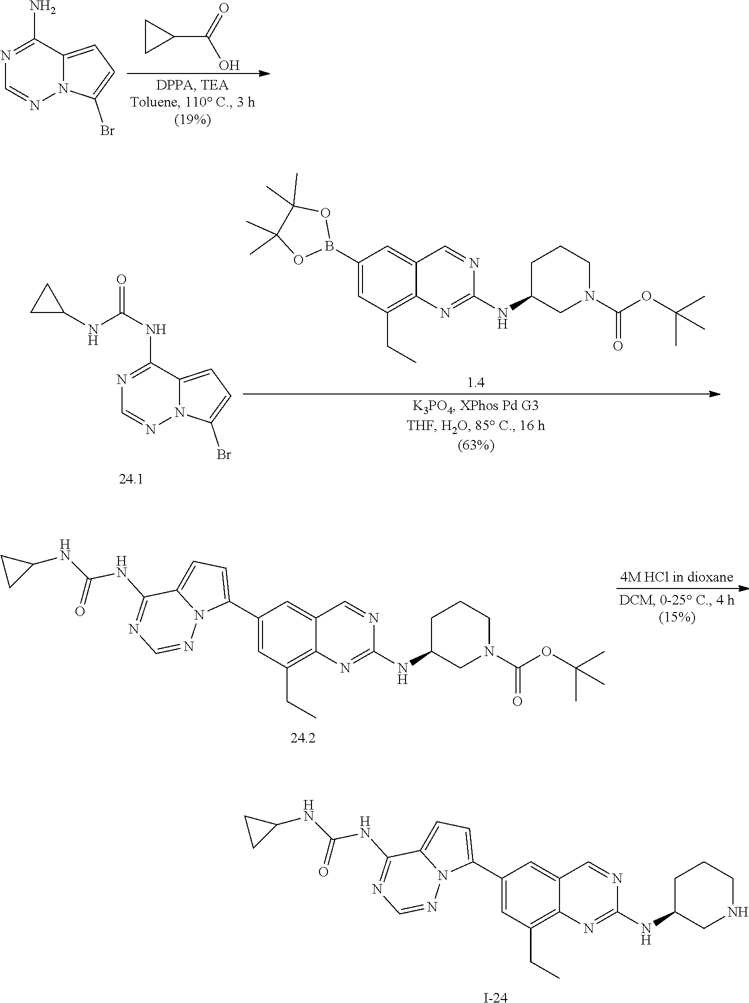

### Synthesis of 24.1.

To a stirred solution of cyclopropionic acid (0.16 mL, 0.002 mmol, 1 eq) and triethylamine (0.58 mL, 0.004 mmol, 2 eq) in toluene (5 mL) was added DPPA (0.67 mL, 0.003 mmol, 1.5 eq) at 25° C. After stirring at 110° C. for 1 h, 7-bromopyrrolo[2,1-f][1,2,4]triazin-4-amine (0.22 mL, 0.001 mmol, 0.5 eq) was added. After stirring at 110° C. for another 2 h, the reaction mixture was concentrated under reduced pressure, quenched with water and extracted with ethyl acetate. The combined organic layer was washed with brine, dried over anhydrous sodium sulfate and concentrated under reduced pressure. The residue was purified by combi-flash column chromatography (RediSepRf 12 g flash column) eluting with 40% ethyl acetate in hexane to afford 24.1 white solid (120 mg, 19%), MS(ES): m/z 296.7 [M+H]⁺, LCMS purity 48%.

### Synthesis of 24.2.

24.2 was synthesized from 24.1 (90 mg, 0.3 mmol, 1 eq) and 1.4 (220 mg, 0.46 mmol, 1.5 eq) using general procedure B to afford 110 mg (Yield: 63%) as white solid. MS(ES): m/z 572.1 [M+H]⁺, LCMS purity 41.5%.

### Synthesis of I-24.

I-24 was synthesized from 24.2 (40 mg, 0.07 mmol, 1 eq) using general procedure C to afforded 4.9 mg (Yield: 15%) as a solid. MS(ES): m/z 472.3 [M+H]⁺, LCMS purity 97.4%, HPLC purity 96.02%, 1H NMR (DMSO-d6, 400 MHz): 10.60-10.50 (m, 1H), 9.60 (bs, 1H), 9.16 (bs, 1H), 8.46 (s, 1H), 8.31 (s, 1H), 8.27 (s, 1H), 8.16 (s, 1H), 7.58 (d, J=4.1 Hz, 1H), 7.38-7.35 (m, 1H), 7.30 (d, J=4.6 Hz, 1H), 4.01 (bs, 1H), 3.03 (d, J=7.6 Hz, 2H), 2.91 (d, J=11.8 Hz, 1H), 2.73 (bs, 1H), 2.65-2.60 (m, 1H), 2.48-2.45 (m, 1H), 1.98 (bs, 1H), 1.89 (s, 1H), 1.74 (bs, 1H), 1.54 (bs, 2H), 1.30 (t, J=7.3 Hz, 3H), 0.74 (d, J=5.6 Hz, 2H), 0.58 (bs, 2H).

### Example 25: Synthesis of (S)-1-(2-chlorophenyl)-N-(7-(8-ethyl-2-(piperidin-3-ylamino)quinazolin-6-yl)pyrrolo[2,1-f][1,2,4]triazin-4-yl)methanesulfonamide. I-25

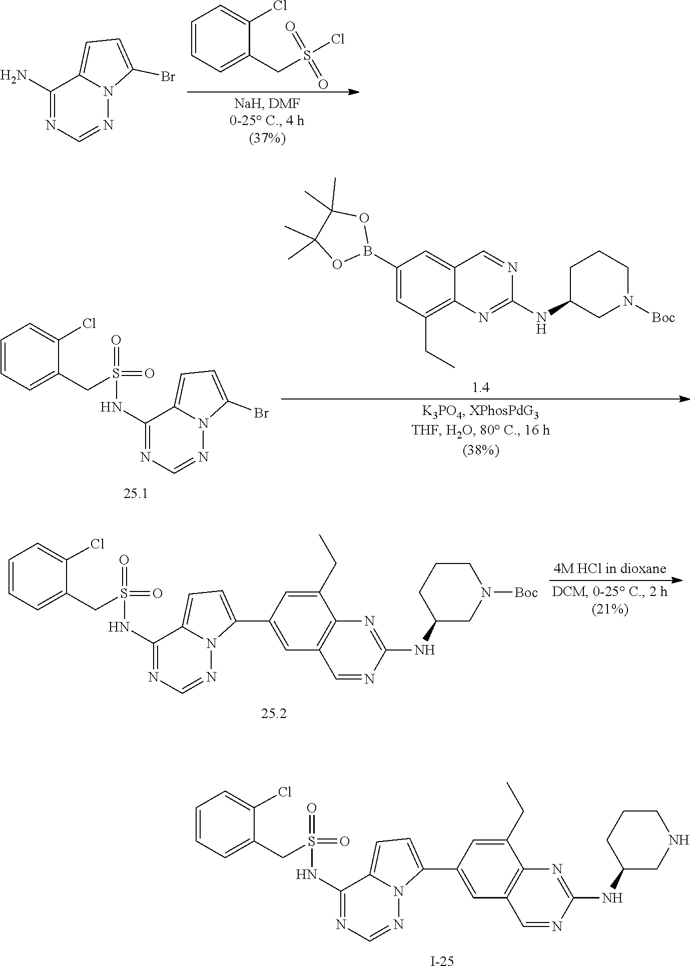

### Synthesis of 25.1.

25.1 was synthesized from 7-bromopyrrolo[2,1-f][1,2,4]triazin-4-amine (100 mg, 0.469 mmol, 1 eq) and (2-chlorophenyl)methanesulfonyl chloride (158 mg, 0.704 mmol, 1.5 eq) using general procedure A to afford 70 mg (Yield: 37%) as white solid. MS(ES): m/z 402.7 [M+H]⁺. LCMS purity 83.13%.

### Synthesis of 25.2.

25.2 was synthesized from 25.1 (70 mg, 0.175 mmol, 1 eq) and 1.4 (100 mg, 0.209 mmol, 1.2 eq) using general procedure B to afford the 45 mg (Yield: 38%) as a light yellow solid. MS(ES): m/z 647.2 [M+H]⁺. LCMS purity 96.33%.

### Synthesis of I-25.

I-25 was synthesized from 25.2 (45 mg, 0.07 mmol, 1 eq) using general procedure C to obtain 8 mg (Yield: 21%) as a solid. MS(ES): m/z 577.2 [M+H]⁺, LCMS purity 98.69%, HPLC purity 95.73%, 1H NMR (DMSO-d6, 400 MHz): 9.19 (s, 1H), 8.52 (bs, 2H), 8.21 (s, 1H), 7.98 (s, 1H), 7.50-7.48 (m, 2H), 7.42-7.40 (m, 1H), 7.29-7.25 (m, 2H), 7.02 (d, J=4.3 Hz, 1H), 6.70 (bs, 1H), 4.77 (s, 2H), 4.24 (bs, 1H), 3.50-3.47 (m, 1H), 3.22-3.19 (m, 1H), 3.07 (q, J=7.4 Hz, 2H), 2.95-2.91 (m, 2H), 2.06-2.03 (m, 1H), 1.95-1.93 (m, 1H), 1.75-1.66 (m, 2H), 1.32 (t, J=7.4 Hz, 3H).

### Example 26: Synthesis of (S)—N-(7-(8-ethyl-2-(piperidin-3-ylamino)quinazolin-6-yl)pyrrolo[2,1-f][1,2,4]triazin-4-yl)benzenesulfonamide. I-26

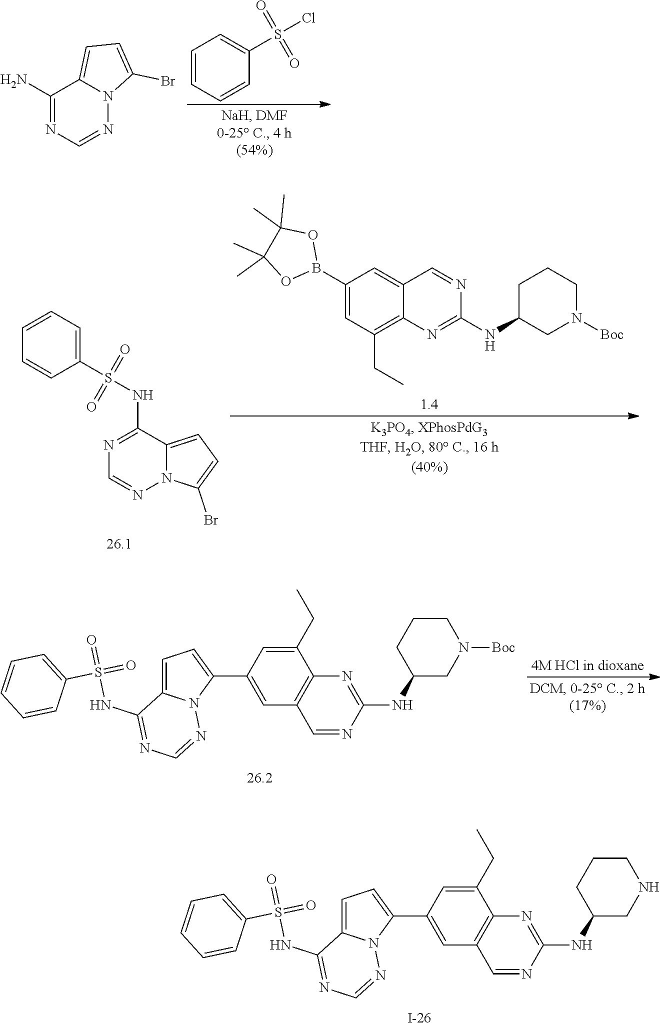

### Synthesis of 26.1.

26.1 was synthesized by using 7-bromopyrrolo[2,1-f][1,2,4]triazin-4-amine (100 mg, 0.469 mmol, 1 eq) and benzenesulfonyl chloride (123 mg, 0.704 mmol, 1.5 eq) using general procedure A to afford 90 mg (Yield: 4%) as white solid. MS(ES): m/z 354.7 [M+H]⁺ LCMS purity 82.13%.

### Synthesis of 26.2.

26.2 was synthesized from 26.1 (70 mg, 0.198 mmol, 1 eq) and 1.4 (114 mg, 0.238 mmol, 1.2 eq) using general procedure B to afford 50 mg (Yield: 40%) as a light yellow solid. MS(ES): m/z 629.3 [M+H]⁺, LCMS purity 95.64%.

### Synthesis of I-26.

I-26 was synthesized using 26.2 (45 mg, 0.072 mmol, 1 eq) using general procedure C to afford 6.67 mg (Yield: 17%) as a solid. LCMS (ES): m/z 529.2 [M+H]⁺, LCMS purity 97.06%, HPLC purity 97.10%, 1H NMR (DMSO-d6, 400 MHz): 9.16 (s, 1H), 8.47 (s, 1H), 8.17-8.13 (m, 2H), 7.89-7.87 (m, 2H), 7.77 (s, 1H), 7.48-7.47 (m, 1H), 7.41-7.40 (m, 3H), 6.98 (d, J=4.2 Hz, 1H), 6.68-6.68 (m, 1H), 4.22 (bs, 1H), 3.49-3.46 (m, 1H), 3.21-3.18 (m, 1H), 3.04 (q, J=7.3 Hz, 2H), 2.92-2.90 (m, 2H), 2.05-2.02 (m, 1H), 1.95-1.92 (m, 1H), 1.74-1.65 (m, 2H), 1.30 (t, J=7.4 Hz, 3H).

### Example 27: Synthesis of (S)—N-(7-(8-ethyl-2-(piperidin-3-ylamino)quinazolin-6-yl)pyrrolo[2,1-f][1,2,4]triazin-4-yl)-4-(trifluoromethoxy)benzenesulfonamide. I-27

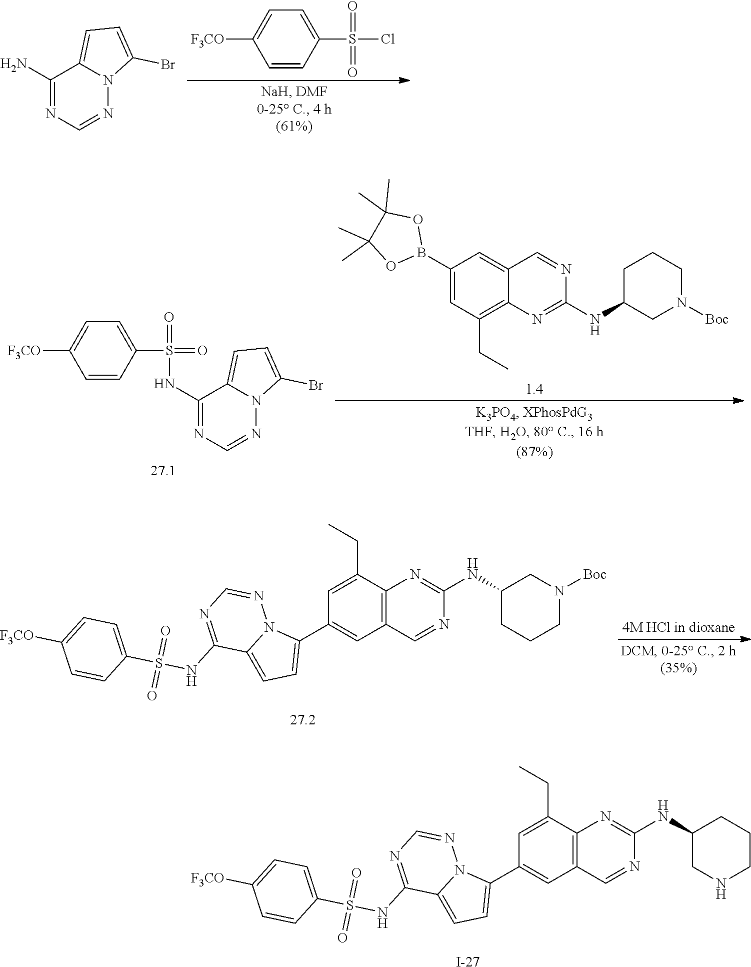

### Synthesis of 27.1.

27.1 was synthesized from 7-bromopyrrolo[2,1-f][1,2,4]triazin-4-amine (100 mg, 0.469 mmol, 1 eq) and 4-(trifluoromethoxy)benzenesulfonyl chloride (123 mg, 0.704 mmol, 1.5 eq) using general procedure A to afford 125 mg (Yield: 61%) as white solid. MS(ES): m/z 354.7 [M+H]⁺, LCMS purity 82.13%.

### Synthesis of 27.2.

27.2 was synthesized using of 27.1 (70 mg, 0.16 mmol, 1 eq) and 1.4 (92 mg, 0.192 mmol, 1.2 eq) using general procedure B to 100 mg (Yield: 87%) as a light yellow solid. MS(ES): m/z 713.2 [M+H]⁺, LCMS purity 99.63%.

### Synthesis of I-27.

I-27 was synthesized from 27.2 (100 mg, 0.14 mmol, 1 eq) using general procedure C to afford 30.1 mg Yield: 35%) as a solid. MS(ES): m/z 613.2 [M+H]⁺, LCMS purity 98.53%, HPLC purity 98.66%, 1H NMR (DMSO-d6, 400 MHz): 9.16 (s, 1H), 8.52 (bs, 1H), 8.47 (s, 1H), 8.17 (s, 1H), 8.00 (d, J=8.6 Hz, 2H), 7.78 (s, 1H), 7.49-7.48 (m, 1H), 7.38 (d, J=8.3 Hz, 2H), 6.99 (d, J=4.4 Hz, 1H), 6.68 (d, J=4.4 Hz, 1H), 4.22 (bs, 1H), 3.48-3.45 (m, 1H), 3.21-3.18 (m, 1H), 3.04 (q, J=7.2 Hz, 2H), 2.92-2.89 (m, 2H), 2.04-2.02 (m, 1H), 1.94-1.91 (m, 1H), 1.73-1.65 (m, 2H), 1.30 (t, J=7.4 Hz, 3H).

### Example 28: Synthesis of N-(7-(2-((2-oxaspiro[3.3]heptan-6-yl)amino)-8-ethylquinazolin-6-yl)pyrrolo[2,1-f][1,2,4]triazin-4-yl)-2-chlorobenzenesulfonamide. I-28

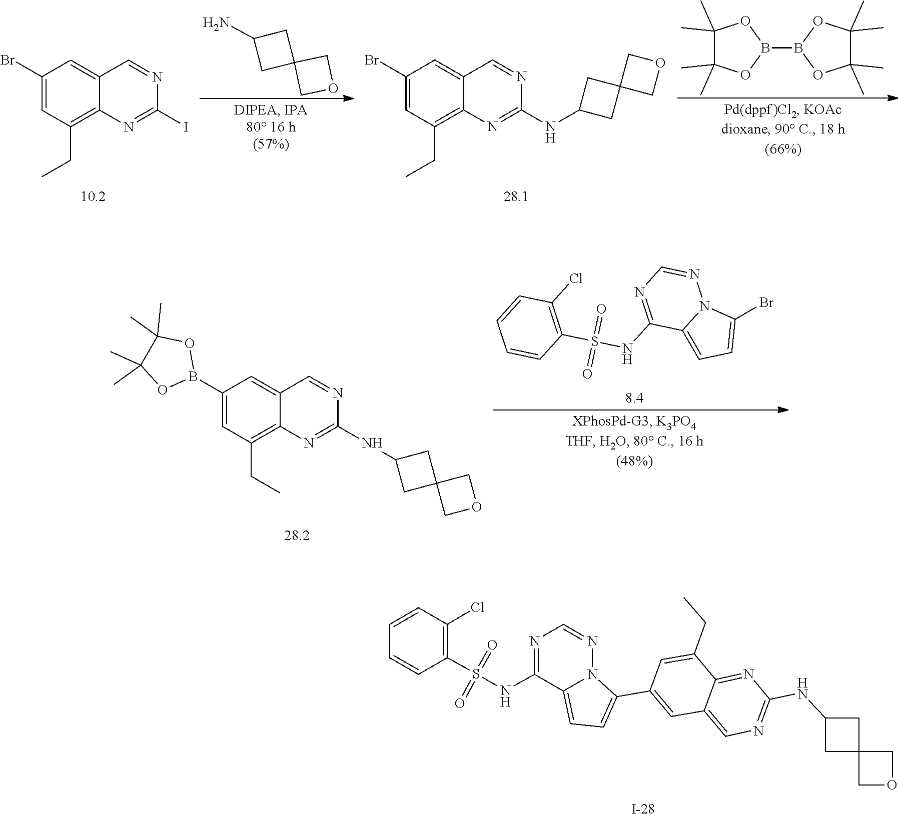

### Synthesis of 28.1.

28.1 was synthesized from 10.2 (200 mg, 0.55 mmol, 1 eq) and 2-oxaspiro[3.3]heptan-6-amine (186 mg, 1.65 mmol, 3 eq) using general procedure H to obtain 100 mg (Yield: 57%) as white solid. MS (ES): m/z 348.0 [M+H]⁺, LCMS purity 99%.

### Synthesis of 28.2.

28.2 was synthesized from 28.1 (100 mg, 0.34 mmol, 1 eq) using general procedure F to obtain 90.2 mg (Yield: 66%) as yellow solid. MS(ES): m/z 396.0 [M+H]⁺, LCMS purity 92%.

### Synthesis of I-28.

I-28 was synthesized from 28.2 (90 mg, 0.21 mmol, 1 eq) and 8.4 (100 mg, 0.25 mmol, 1 eq) using general procedure B to obtain 15.1 mg (Yield: 12.8%) as yellow solid. MS(ES): m/z 576.5 [M+H]⁺, LCMS purity 97%, 1H NMR (DMSO-d6, 400 MHz): 9.12 (bs, 1H), 8.33 (s, 1H), 8.16 (d, J=7.6 Hz, 1H), 8.09 (d, J=12.9 Hz, 2H), 7.78 (bs, 1H), 7.65-7.62 (m, 2H), 7.57 (t, J=6.2 Hz, 1H), 7.26 (s, 1H), 7.18 (d, J=4.5 Hz, 1H), 4.66 (s, 2H), 4.53 (s, 2H), 4.22 (bs, 1H), 2.99 (q, J=7.2 Hz, 2H), 2.67-2.65 (m, 2H), 2.31-2.27 (m, 2H), 1.30 (t, J=7.3 Hz, 3H).

### Example 29: Synthesis of 2-chloro-N-(7-(8-ethyl-2-(((1r,4r)-4-hydroxycyclohexyl)amino)quinazolin-6-yl)pyrrolo[2,1-f][1,2,4]triazin-4-yl)benzenesulfonamide. I-29

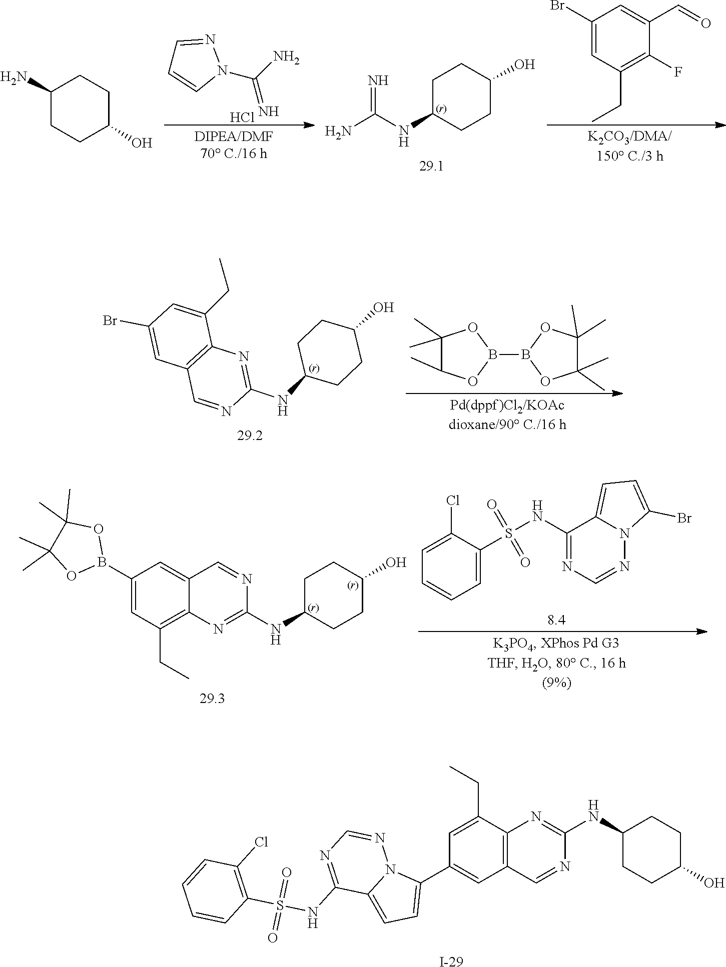

### Synthesis of 29.1.

29.1 was synthesized from (1r,4r)-4-aminocyclohexan-1-ol (500 mg, 4.34 mmol, 1 eq) and 1H-pyrazole-1-carboximidamide hydrochloride (958.69 mg, 6.5 mmol, 1.5 eq) using general procedure D to obtain 550 mg (Yield: 73%) as white solid. GC-MS (ES): m/z 157.2 [M+H]⁻.

### Synthesis of 29.2.

29.2 was synthesized from 1.1 and (200 mg, 0.86 mmol, 1 eq) and 29.1 (270 mg, 1.72 mmol, 2 eq) using general procedure E to afford 120 mg (Yield: 39%) as an brown solid. MS(ES): m/z 350.2[M+H]⁺, LCMS purity 92.54%.

### Synthesis of 29.3.

29.3 was synthesized from 29.2 (120 mg, 0.34 mmol, 1 eq) using general procedure F to obtain 105 mg (Yield: 77%) as a brown solid MS(ES): m/z 398.5 [M+H]⁺, LCMS purity 92.01%.

### Synthesis of I-29.

I-29 was synthesized from 8.4 (100 mg, 0.15 mmol, 1 eq) and 29.3 (123 mg, 0.31 mmol, 1.2 eq) using general procedure B to obtain 5.08 mg (Yield: 9%) as a solid. MS(ES): m/z 578.1 [M+H]⁺, LCMS purity 98.65%, HPLC purity 98.96%, 1H NMR (DMSO-d6, 400 MHz): 12.04 (s, 1H), 9.10 (s, 1H), 8.32 (s, 1H), 8.15 (d, J=7.7 Hz, 1H), 8.07 (d, J=15.68 Hz, 2H), 7.63-7.54 (m, 3H), 7.38 (bs, 1H), 7.24 (bs, 1H), 7.17 (d, J=3.5 Hz, 1H), 4.53 (d, J=3.8 Hz, 1H), 3.79 (bs, 1H), 3.43 (bs, 1H), 3.04-2.98 (m, 2H), 2.01 (bs, 2H), 1.88 (d, J=11.24 Hz, 2H), 1.41-1.23 (m, 6H).

### Example 30: Synthesis of Pyrrolidine-1-sulfonic acid {7-[8-ethyl-2-(piperidin-3-ylamino)-quinazolin-6-yl]-pyrrolo[2,1-f][1,2,4]triazin-4-yl}-amide, I-30

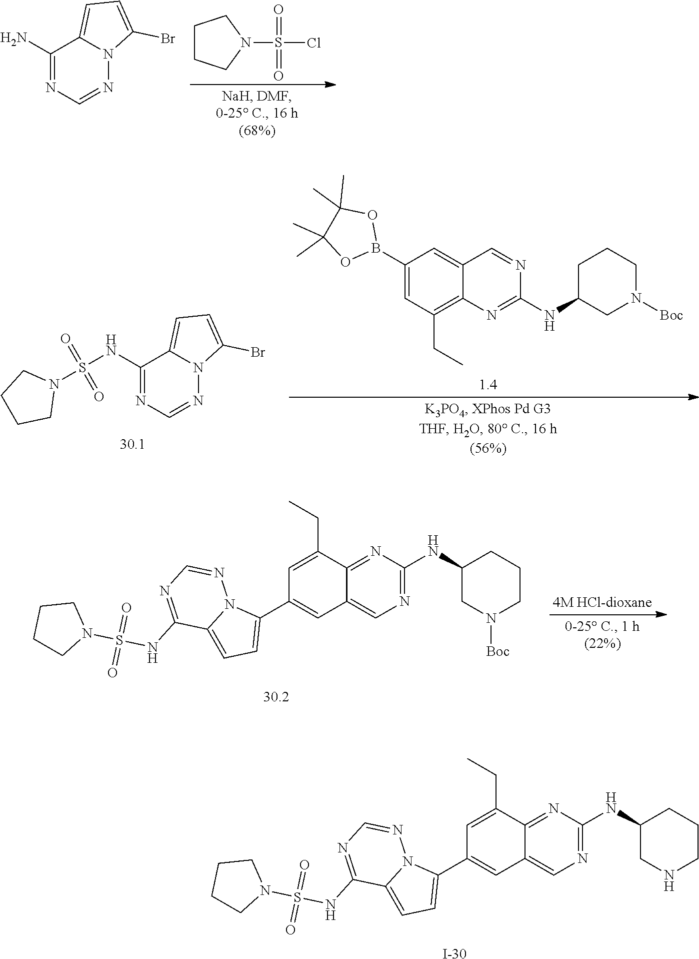

### Synthesis of 30.1.

30.1 was synthesized from of 7-bromopyrrolo[2,1-f][1,2,4]triazin-4-amine (100 mg, 0.47 mmol, 1 eq) and pyrrolidine-1-sulfonyl chloride (119 mg, 0.70 mmol, 1.5 eq) using general procedure A to afford 110 mg (Yield: 68%) as a yellow gum. MS (ES): m/z 345 [M+H]⁺, LCMS purity 85.57%.

### Synthesis of 30.2.

30.2 was synthesized from 30.1 (100 mg, 0.28 mmol, 1 eq) and 1.4 (146 mg, 0.34 mmol, 1.2 eq) using general procedure B to afford 95 mg (Yield: 56%) as a yellow gum. MS (ES): m/z 622.0 [M+H]⁺, LCMS purity 99.35%.

### Synthesis of I-30.

I-30 was synthesized from 30.2 (95 mg, 0.15 mmol, 1 eq) using general procedure C to afford 17.7 mg (Yield: 22%) as a solid. MS (ES): m/z 522.2 [M+H]⁺, LCMS purity 99.53%, HPLC purity 98.59%, 1H NMR (DMSO-d6, 400 MHz): 9.16 (s, 1H), 8.49 (s, 1H), 8.18 (s, 1H), 7.85 (s, 1H), 7.46-7.44 (m, 1H), 6.97 (d, J=4.3 Hz, 1H), 6.68 (d, J=4.1 Hz, 1H), 4.19 (bs, 1H), 3.44-3.41 (m, 2H), 3.24-3.13 (m, 5H), 3.08-3.02 (m, J=9.6 Hz, 2H), 2.87-2.84 (m, 2H), 2.04-1.89 (m, 2H), 1.80-1.77 (m, 3H), 1.71-1.64 (m, 2H), 1.31 (t, J=7.4 Hz, 3H).

### Example 31: Synthesis of 2-chloro-N-(7-(8-ethyl-2-(((3S,5S)-5-fluoropiperidin-3-yl)amino)quinazolin-6-yl)pyrrolo[2,1-f][1,2,4]triazin-4-yl)benzenesulfonamide. I-31

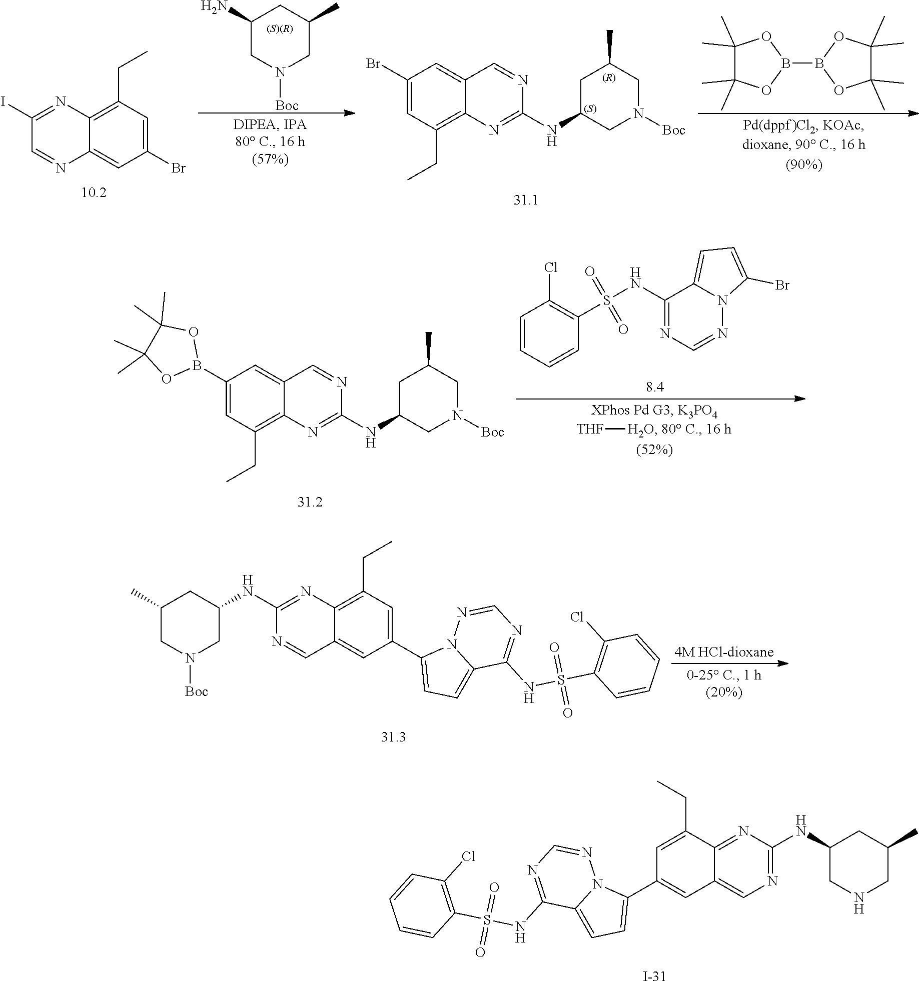

### Synthesis of 31.1.

31.1 was synthesized from 10.2 (200 mg, 0.55 mmol, 1 eq) and tert-butyl (3S,5S)-3-amino-5-fluoropiperidine-1-carboxylate (250 mg, 1.65 mmol, 3 eq) using general procedure H to obtain 100 mg (Yield: 57%) as yellow solid. MS(ES): m/z 452.7 [M+H]⁺, LCMS purity 97.1%.

### Synthesis of 31.2.

31.2 was synthesized from 31.1 (121 mg, 0.34 mmol, 1 eq) using general procedure B obtain 100 mg (Yield: 90.6%) as solid. MS(ES): m/z 449.2 [M+H]⁺, LCMS purity 92%.

### Synthesis of 31.3.

31.3 was synthesized from 31.2 (100 mg, 0.23 mmol, 1 eq) and 8.4 (107 mg, 0.24 mmol, 1 eq) using general procedure C to obtain 80 mg (Yield: 52%) as white solid. MS(ES): m/z 677.5 [M+H]⁺, LCMS purity 93%.

### Synthesis of I-31.

I-31 was synthesized from 31.3 (70 mg, 0.22 mmol, 1 eq) using general procedure C to obtain 12.4 mg (Yield: 20%) as a solid. MS(ES): m/z 575.2 [M−H]⁻, LCMS purity 100%, HPLC purity 99.23%, 1H NMR (DMSO-d6, 400 MHz): 9.15 (s, 1H), 8.73-8.70 (m, 1H), 8.47 (s, 1H), 8.18 (s, 1H), 8.06 (d, J=2.7 Hz, 1H), 7.66 (s, 1H), 7.49 (bs, 1H), 7.40 (s, 3H), 6.99 (d, J=4.1 Hz, 1H), 6.70 (d, J=4.0 Hz, 1H), 4.24 (bs, 1H), 3.56 (bs, 1H), 3.11 (d, J=10.8 Hz, 1H), 3.05 (d, J=7.1 Hz, 2H), 2.65 (t, J=11.8 Hz, 1H), 2.09-1.94 (m, 2H), 1.30 (t, J=7.5 Hz, 3H), 1.28-1.22 (m, 1H), 0.96 (d, J=6.3 Hz, 3H).

### Example 32: Synthesis of (N-(7-(2-(((1r,4r)-4-aminocyclohexyl)amino)-8-ethylquinazolin-6-yl)pyrrolo[2,1-f][1,2,4]triazin-4-yl)-2-chlorobenzenesulfonamide. I-32

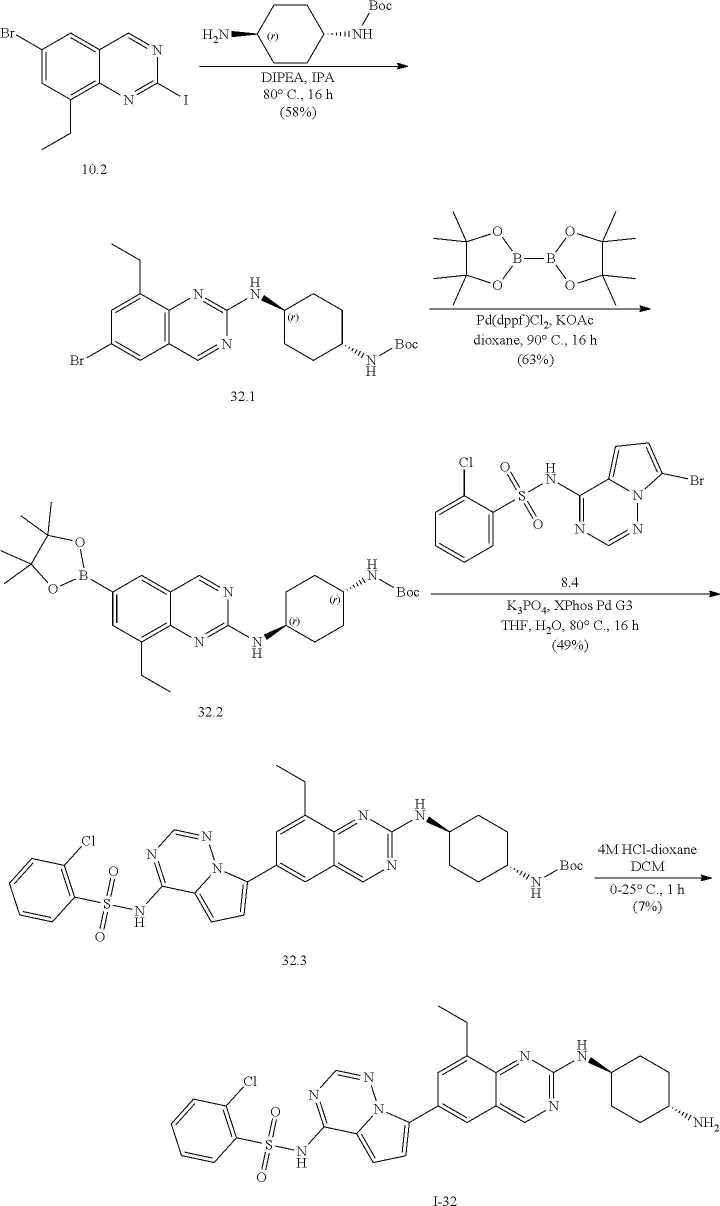

### Synthesis of 32.1.

32.1 was synthesized from 10.2 (150 mg, 0.41 mmol, 1 eq) and tert-butyl 3-aminoazetidine-1-carboxylate (177 mg, 0.82 mmol, 2 eq) using general procedure H to obtain 150 mg (Yield: 81%) as yellow solid. MS(ES): m/z 451.2 [M+H]⁺, LCMS purity 94.37%.

### Synthesis of 32.2.

32.2 was synthesized from 32.1 (150 mg, 0.33 mmol, 1 eq) using general procedure D to obtain 160 mg (Yield: 96%) as a light yellow solid. MS(ES): m/z 497.1[M+H]⁺, LCMS purity 73.63%.

### Synthesis of 32.3.

32.3 was synthesized from 8.4 (200 mg, 0.51 mmol, 1.5 eq) and 32.2 (170 mg, 0.35 mmol, 1 eq) using general procedure B to obtain 90 mg (Yield: 40%) as a light yellow solid. MS(ES): m/z 677.0[M+H]⁺, LCMS purity 77.63%.

### Synthesis of I-32.

I-32 was synthesized from 32.3 (80 mg, 0.11 mmol, 1 eq) using general procedure C to obtain 3.5 mg (Yield: 7%) as a solid. MS(ES): m/z 577.2 [M+H]⁺, LCMS purity 97.96%, HPLC purity 98.54%, 1H NMR (DMSO-d6, 400 MHz): 9.09 (s, 1H), 8.42 (s, 1H), 8.15 (d, J=14.4 Hz, 1H), 8.06 (bs, 1H), 7.70-7.64 (m, 3H), 7.38 (d, J=2.8 Hz, 4H), 6.97 (d, J=4.4 Hz, 1H), 6.68 (d, J=4.3 Hz, 1H), 3.81 (bs, 1H), 3.03-2.99 (m, 3H), 2.10 (bs, 2H), 2.01-1.98 (m, 2H), 1.47-1.37 (m, 4H), 1.30 (t, d=7.0 Hz, 3H).

### Example 33: Synthesis of (S)—N-(7-(8-ethyl-2-(piperidin-3-ylamino)quinazolin-6-yl)pyrrolo[2,1-f][1,2,4]triazin-4-yl)propane-1-sulfonamide. I-33

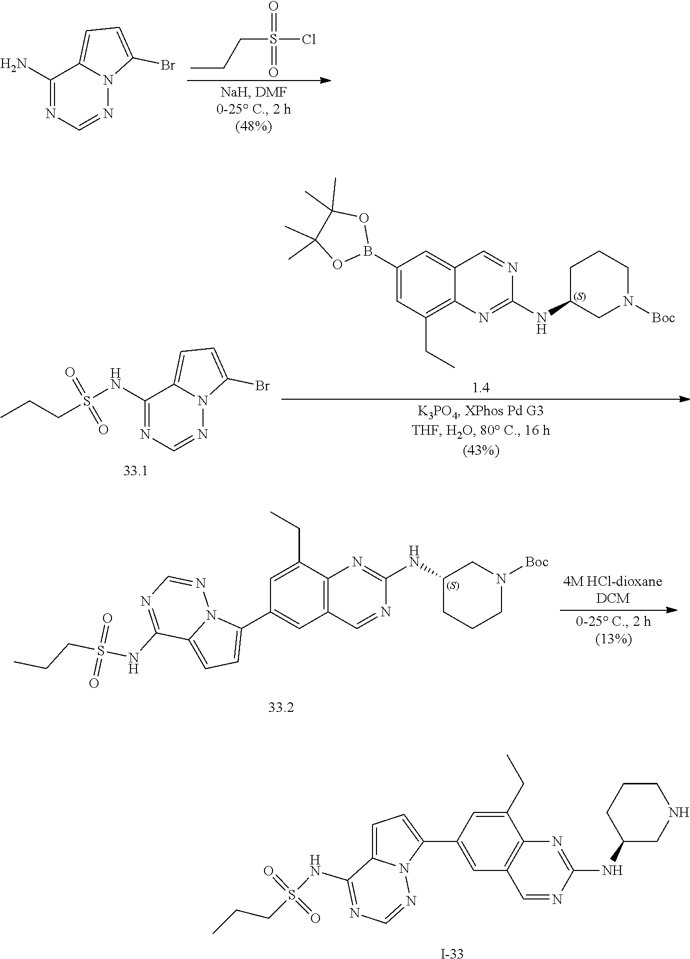

### Synthesis of 33.1.

33.1 was synthesized from 7-bromopyrrolo[2,1-f][1,2,4]triazin-4-amine (150 mg, 0.70 mmol, 1 eq) and propane-1-sulfonyl chloride (0.15 mL, 1.05 mmol, 1.5 eq) using general procedure A to obtain 110 mg (Yield: 48%) as light brown solid. MS(ES): m/z 318.9[M+H]⁺, LCMS purity 91%.

### Synthesis of 33.2.

33.2 was synthesized from 33.1 (100 mg, 0.31 mmol, 1 eq) and 1.4 (302 mg, 0.63 mmol, 2 eq) using general procedure B to obtain 80 mg (Yield: 43%) as an off-white solid. MS(ES): m/z 595.2[M+H]⁺, LCMS purity 87.63%.

### Synthesis of I-33.

I-33 was synthesized from 33.2 (80 mg, 0.13 mmol, 1 eq) using general procedure C to obtain 11.53 mg (Yield: 13%) as a solid. MS(ES): m/z 495.5 [M+H]⁺, LCMS purity 100%, HPLC purity 98.54%, 1H NMR (DMSO-d6 at 20° C., 400 MHz): 9.18 (s, 1H), 8.51 (s, 1H), 8.17 (d, J=20.6 Hz, 1H), 7.89 (s, 1H), 7.49 (bs, 1H), 6.98 (d, J=4.3 Hz, 1H), 6.65 (d, J=4.1 Hz, 1H), 4.23 (bs, 1H), 3.49-3.46 (m, 1H), 3.25-3.21 (m, 3H), 3.08-3.03 (m, 2H), 2.92-2.90 (m, 2H), 2.06-2.04 (m, 1H), 1.93-1.91 (m, 1H), 1.74-1.62 (m, 4H), 1.31 (t, d=7.4 Hz, 3H). 0.93 (t, d=7.4 Hz, 3H).

### Example 34: Synthesis of (S)-2-chloro-N-(7-(8-ethyl-2-(piperidin-3-ylamino)quinazolin-6-yl)pyrrolo[2,1-f][1,2,4]triazin-4-yl)-4-(trifluoromethyl)benzenesulfonamide. I-34

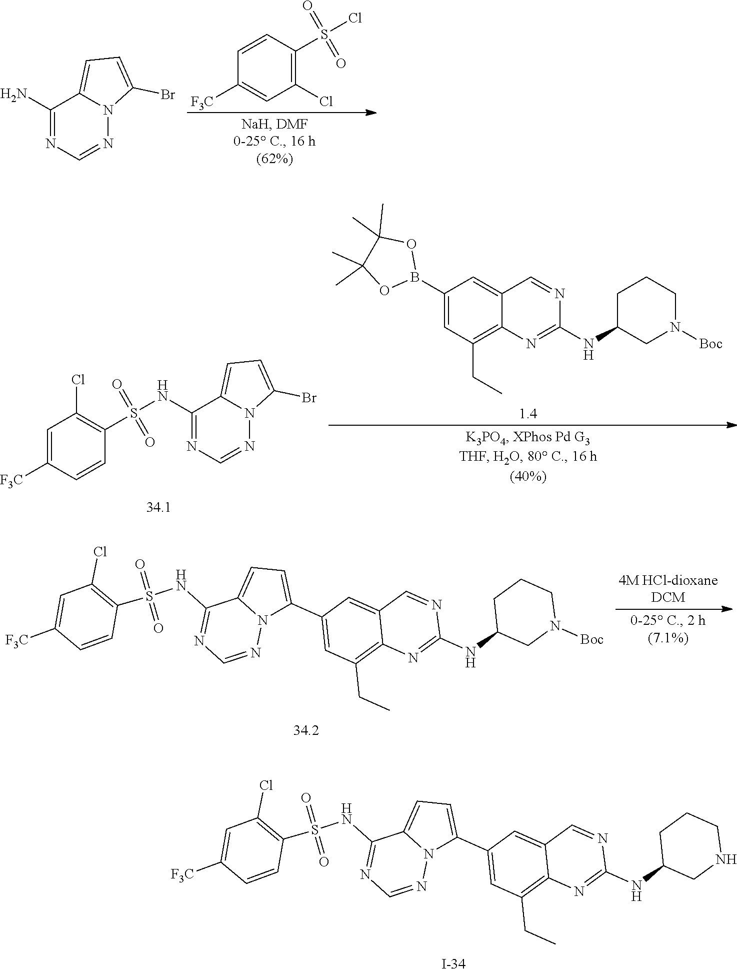

### Synthesis of 34.1.

34.1 was synthesized from 7-bromopyrrolo[2,1-f][1,2,4]triazin-4-amine (150 mg, 0.70 mmol, 1.0 eq) and 2-chloro-4-(trifluoromethyl)benzenesulfonyl chloride (294 mg, 1.05 mmol, 1.5 eq) using general procedure A to obtain 200 mg (Yield: 62%) as light brown solid. MS(ES): m/z 455.1[M+H]⁺, LCMS purity 94%.

### Synthesis of 34.2.

34.2 was synthesized from 34.1 (100 mg, 0.31 mmol, 1 eq) and 1.4 (302.30 mg, 0.63 mmol, 2 eq) using general procedure B to obtain 70 mg (Yield: 40%) as an off-white solid. MS(ES): m/z 731.2 [M+H]⁺, LCMS purity 90.1%.

### Synthesis of I-34.

I-34 was synthesized from 34.2 (70 mg, 0.154 mmol, 1 eq) using general procedure C to obtain 8.1 mg (Yield: 7.1%) as a solid. MS(ES): m/z 631.2 [M+H]⁺, LCMS purity 99.59%, HPLC purity 99.47%, 1H NMR (DMSO-d6, 400 MHz): 9.61 (s, 1H), 9.19 (s, 1H), 8.39 (d, J=8.5 Hz, 1H), 8.33 (s, 1H), 8.15 (d, J=3.8 Hz, 2H), 7.80 (d, J=8.4 Hz, 1H), 7.48-7.43 (bs, 2H), 7.33 (d, J=4.4 Hz, 1H), 7.20 (d, J=4.3 Hz, 1H), 3.90 (bs, 1H), 3.07 (t, J=7.2 Hz, 2H), 1.99-1.64 (m, 3H), 1.43-1.40 (m, 2H) 1.34 (t, J=7.2 Hz, 3H), 1.23-1.14 (m, 3H).

### Example 35: Biological Assays

### IRE1a Biochemical Enzyme Splice Assay:

A truncated human IRE1a WT protein, containing the catalytic part (aa 547-977) was expressed in baculovirus. The purified protein (20 nM) was incubated with a fluorescently quenched IRE1a substrate (500 nM; 5′/6-FAM-CAGUCCGCAGCACUG-BHQ-1/3′) and a titration of inhibitor, starting at 10 μM (final DMSO concentration=1%) for 40 min at room temperature in assay buffer. Reactions were carried out in a final volume of 20 μM in a 384 well assay plate. The reaction was stopped, and the plate was spun for 1 minute at 1000 rpm. The fluorescence intensity of the IRE1a-substrate was measured using an fluorescent plate reader (Ex 485 nm/Em 535 nm, Perkin Elmer Envision).

Results of the IRE1a biochemical enzyme splice assay are listed in Table 2, below. Compounds designated as “A” had an IC₅₀ under 100 nM. Compounds designated as “B” had an IC₅₀ between 100 nM and 1 μM. Compounds designated as “C” had an IC₅₀ greater than 1 μM.

### XBP1-Luc Cell Assay:

HEK293-pLVX-XBP1.fluc-IRES-Puro cells (ChemPartner) were collected into DMEM (Invitrogen, 11995-065) with 10% FBS (BI, 04-002-1A). Cells were seeded at 1.5E4 cells/well in BioCoat Poly-D-Lysine Black/Clear flat bottom 96 well plate (Corning, 356640) and and incubated overnight at 37° C. and 5% CO₂. Cells were treated with 600 nM Thapsigargin (Sigma, T9033) for 1 hour at 37° C. and 5% CO₂ prior to inhibitor addition. A titration of inhibitor, starting at 30 μM was added to wells for an additional 6 hours at 37° C. and 5% CO₂. 100 μl of Steady-Glo Luciferase assay reagent (Promega, E2550) was added to plate and the plate was shaken for 2 minutes. Signal was read using EnSpire (Perkin Elmer).

Results of the XBP-1-Luc cell assay are listed in Table 2, below. Compounds designated as “A” had an IC₅₀ under 1 μM. Compounds designated as “B” had an IC₅₀ between 1 μM and 10 μM. Compounds designated as “C” had an IC₅₀ greater than 10 μM.

TABLE 2
IRE1a biochemical enzyme splice assay and XBP1-Luc cell assay results.

| | EnzymeSplice_hIRE1a_IC₅₀ | fLuc_XBP1_hIRE1a_HEK293_IC₅₀ |
|---|---|---|
| Compound | (IC₅₀) | (IC₅₀) |
| I-1 | A | A |
| I-2 | A | A |
| I-3 | A | A |
| I-4 | B | C |
| I-5 | A | B |
| I-6 | A | A |
| I-7 | A | A |
| I-8 | A | B |
| I-9 | A | B |
| I-10 | C | |
| I-11 | A | B |
| I-12 | A | A |
| I-13 | C | |
| I-14 | A | C |
| I-15 | A | A |
| I-16 | | |
| I-17 | B | C |
| I-18 | B | C |
| I-19 | A | A |
| I-20 | B | B |
| I-21 | A | A |
| I-22 | A | A |
| I-23 | A | B |
| I-24 | B | A |
| I-25 | A | A |
| I-26 | A | B |
| I-27 | B | B |
| I-28 | B | C |
| I-29 | A | B |
| I-30 | A | A |
| I-31 | A | A |
| I-32 | A | A |
| I-33 | A | A |
| I-34 | C | |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of formula I′: 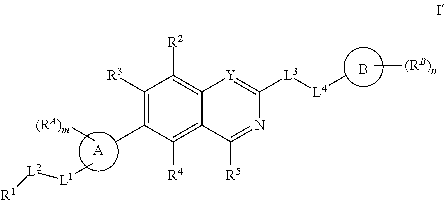
or a pharmaceutically acceptable salt thereof, wherein:
Ring A is a 7-12-membered fused bicyclic saturated, partially unsaturated, or unsaturated, carbocyclic, aryl, heterocyclic, or heteroaryl ring system, having 0-5 heteroatoms independently selected from nitrogen, oxygen, and sulfur;
L¹ is —O—, —S—, —NR—, —S(O)₂—, —S(O)₂NR—, —S(O)—, —S(O)NR—, —C(O)—, —C(O)O—, —C(O)NR—, —N(R)C(O)N(R)—, —C(O)N(R)O—, —OC(O)—, —OC(O)NR—, —N(R)C(O)O—, —N(R)C(O)—, —N(R)S(O)₂—; or L¹ is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
L² is a covalent bond or L² is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
R¹ is selected from C₁₋₆ aliphatic; phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; and a 3-7 membered saturated or partially unsaturated monocyclic heterocyclic ring having 1-2 heteroatoms independently selected from nitrogen, oxygen, and sulfur; each of which is substituted with q instances of R^{C};
each instance of R^{A} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{A} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D};
Y is N or CR;
each of R², R³, R⁴, and R⁵, is independently selected from hydrogen, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, halogen, an optionally substituted C₁₋₆ aliphatic; optionally substituted phenyl; optionally substituted naphthalenyl; an optionally substituted 3-7 membered saturated or partially unsaturated monocyclic carbocyclic ring; an optionally substituted 3-7 membered saturated or partially unsaturated monocyclic heterocyclic ring having 1-2 heteroatoms independently selected from nitrogen, oxygen, phosphorous, silicon and sulfur; and an optionally substituted 5-6 membered monocyclic heteroaryl ring having 1-4 heteroatoms independently selected from nitrogen, oxygen, and sulfur; each of which is substituted with r instances of R and s instances of R^{D};
Ring B is selected from phenyl; a 3-7 membered saturated or partially unsaturated monocyclic carbocyclic ring; a 5-6 membered monocyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; and a 6-10 membered saturated or partially unsaturated spirocyclic, fused, or bridged ring having 0-3 heteroatoms independently selected from nitrogen, oxygen, and sulfur; each of which is substituted with q instances of R^{C};
L³ is —O—, —S—, —NR—, —S(O)₂—, —S(O)₂NR—, —S(O)—, —S(O)NR—, —C(O)—, —C(O)O—, —C(O)NR—, —C(O)N(R)O—, —OC(O)—, —OC(O)NR—, —N(R)C(O)O—, —N(R)C(O)N(R)—, —N(R)C(O)—, —N(R)S(O)₂—; or L³ is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
L⁴ is a covalent bond or L⁴ is a C₁₋₄ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—;
each instance of R^{B} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{B} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D};
each instance of R^{C} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂; or each instance of R^{C} is independently an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; or a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; or a 6-11 membered saturated or partially unsaturated bicyclic heterocyclic ring having 1-2 heteroatoms independently selected from nitrogen, oxygen, and sulfur; each of which is substituted with r instances of R and s instances of R^{D};
each instance of R^{D} is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)NR₂, —N(R)S(O)₂NR₂, —N(R)S(O)₂R, —N═S(O)R₂, —S(NR)(O)R, —N(R)S(O)R, —N(R)CN, —P(O)(R)NR₂, —P(O)(R)OR or —P(O)R₂;
each R is independently hydrogen, —CN, halogen, or an optionally substituted group selected from C₁₋₆ aliphatic; phenyl; naphthalenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same nitrogen are taken together with the nitrogen to form an optionally substituted 4-7 membered monocyclic saturated, partially unsaturated, or heteroaryl ring having, in addition to the nitrogen, 0-3 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur;
m is 0, 1, 2, 3, or 4;
n is 0, 1, 2, 3, or 4;
each q is independently 0, 1, 2, 3, or 4;
each r is independently 0, 1, 2, 3, or 4; and
each s is independently 0, 1, 2, 3, or 4.

2-3. (canceled)

4. The compound of claim 1, or a pharmaceutically acceptable salt thereof, wherein Ring A is

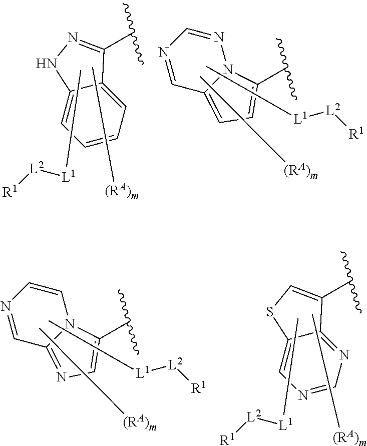

5. (canceled)

6. The compound of claim 1, or a pharmaceutically acceptable salt thereof, wherein L¹ is —NR—, —N(R)C(O)N(R)—, or —N(R)S(O)₂—.

7. The compound of claim 1, or a pharmaceutically acceptable salt thereof, wherein L² is a covalent bond or L² is a C₁₋₄ bivalent saturated straight hydrocarbon chain wherein one or two methylene units of the chain are optionally and independently replaced by —C(R)₂—, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—.

8-9. (canceled)

10. The compound of claim 1, or a pharmaceutically acceptable salt thereof, wherein R¹ is selected from -Et, —Pr, —CH₂CF₃,

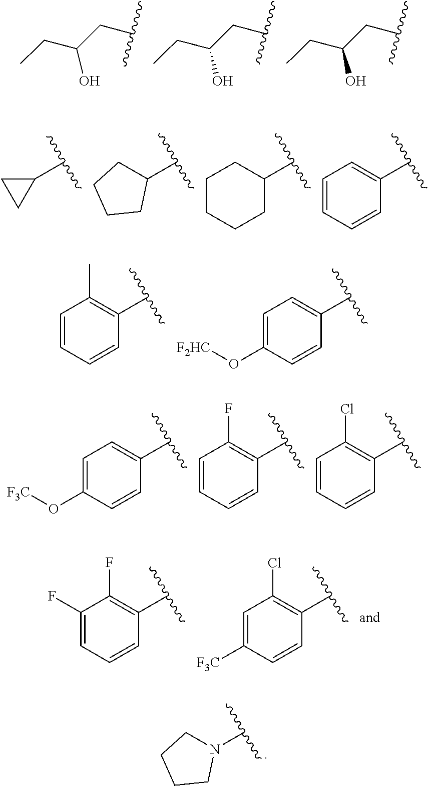

11. The compound of claim 1, or a pharmaceutically acceptable salt thereof, wherein Ring A, L¹, L², R¹, and R^{A}, together, are selected from

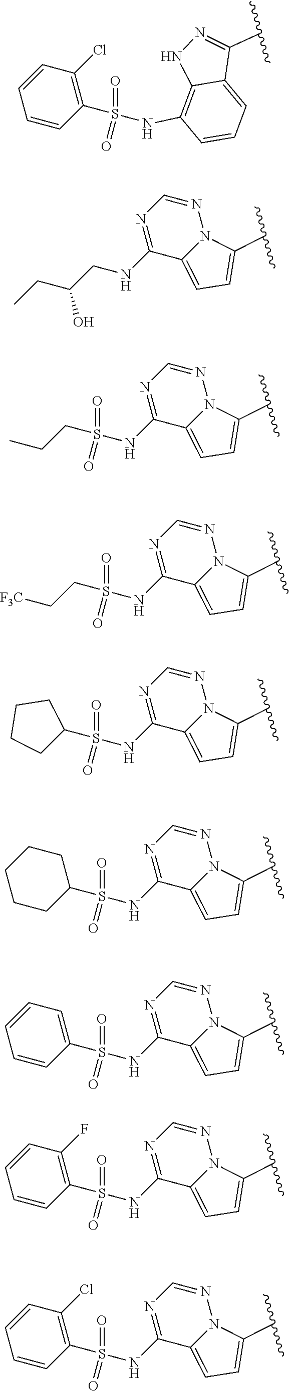

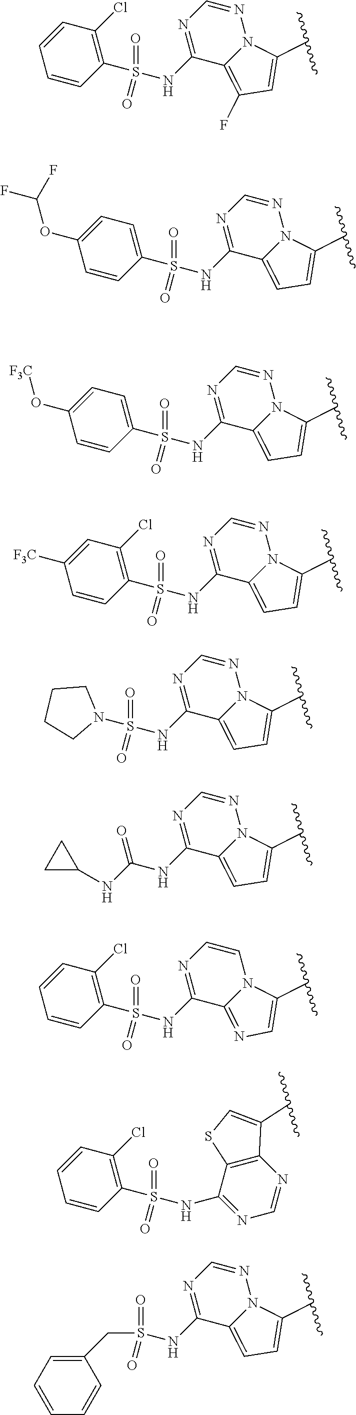

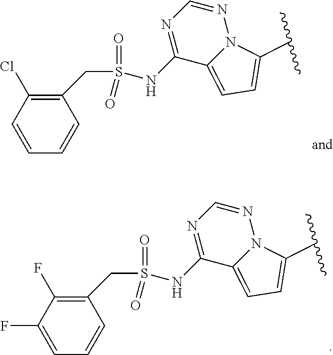 [SMILES: *c1ccc2c(NS(=O)(=O)Cc3ccccc3Cl)ncnn12 ; *c1ccc2c(NS(=O)(=O)Cc3cccc(F)c3F)ncnn12]

12-13. (canceled)

14. The compound of claim 1, or a pharmaceutically acceptable salt thereof, wherein L⁴ is a covalent bond or L⁴ is a C₁₋₂ bivalent saturated or unsaturated, straight or branched hydrocarbon chain wherein one or two methylene units of the chain are optionally and independently replaced by —C(R)₂—, —N(R)—, —N(R)C(O)N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)— or —S(O)₂—.

15. (canceled)

16. The compound of claim 1, or a pharmaceutically acceptable salt thereof, wherein Ring B is selected from

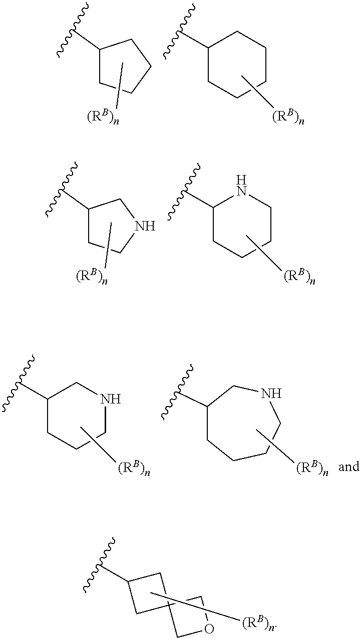

17. The compound of claim 1, or a pharmaceutically acceptable salt thereof, wherein L³, L⁴, Ring B, and R^{B} is

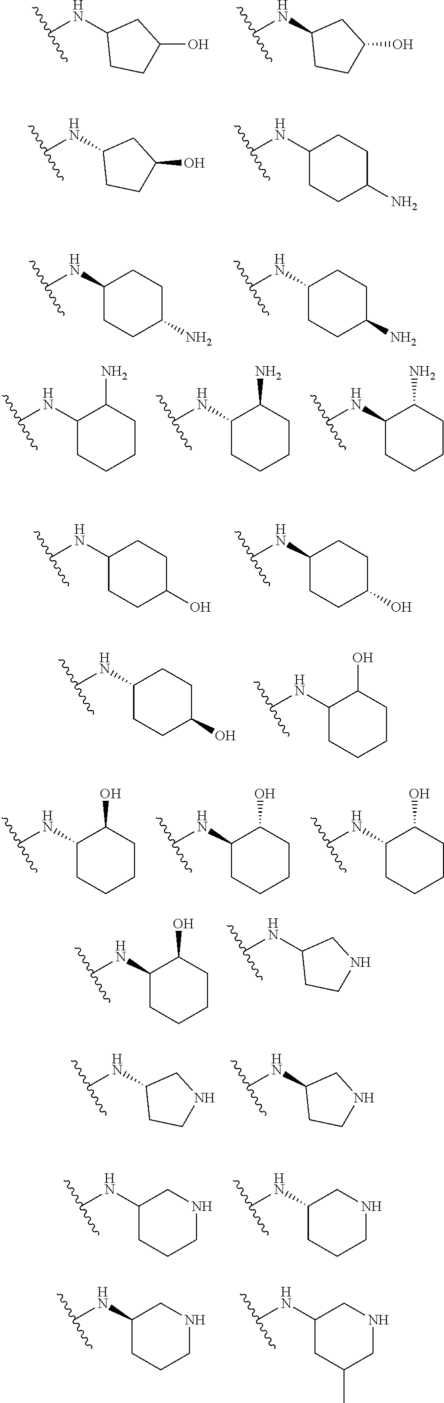

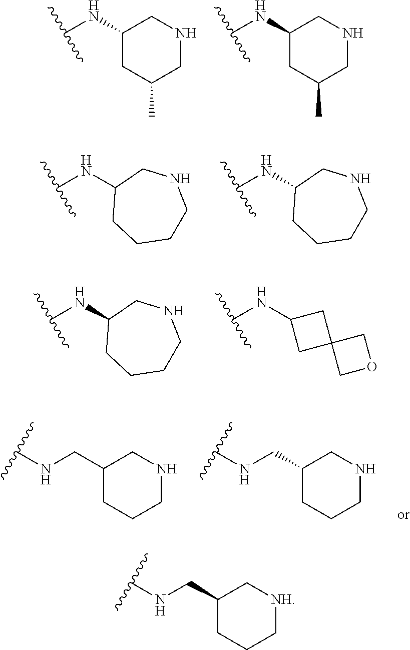

18. The compound of claim 1, wherein the compound is of formula I-a: 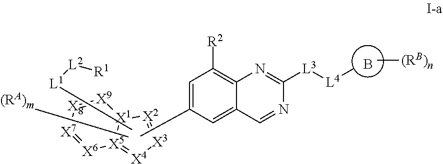
or a pharmaceutically acceptable salt thereof.

19. The compound of claim 1, wherein the compound is selected from

| Compound | Structure |
|---|---|
| I-1 | 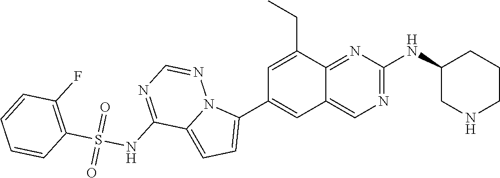 [SMILES: CCc1cc(-c2ccc3c(NS(=O)(=O)c4ccccc4F)ncnn23)cc2cnc(N[C@H]3CCCNC3)nc12] |
| I-2 | 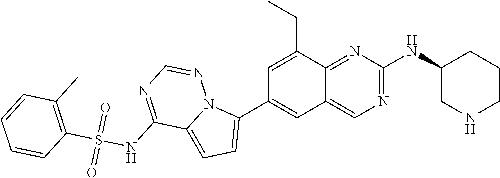 [SMILES: CCc1cc(-c2ccc3c(NS(=O)(=O)c4ccccc4C)ncnn23)cc2cnc(N[C@H]3CCCNC3)nc12] |
| I-3 | 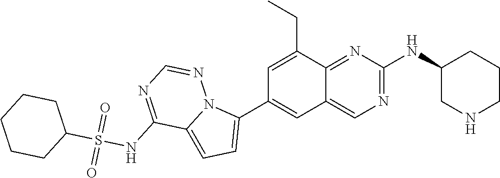 [SMILES: CCc1cc(-c2ccc3c(NS(=O)(=O)C4CCCCC4)ncnn23)cc2cnc(N[C@H]3CCCNC3)nc12] |
| I-4 | 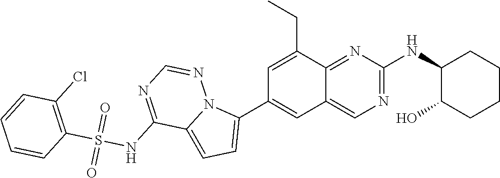 [SMILES: CCc1cc(-c2ccc3c(NS(=O)(=O)c4ccccc4Cl)ncnn23)cc2cnc(N[C@H]3CCCC[C@@H]3O)nc12] |
| I-5 | 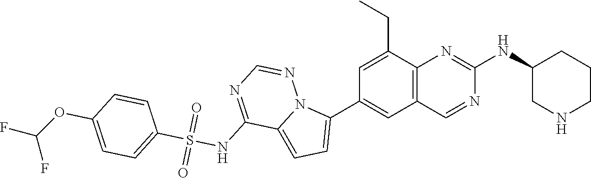 [SMILES: CCc1cc(-c2ccc3c(NS(=O)(=O)c4ccc(OC(F)F)cc4)ncnn23)cc2cnc(N[C@H]3CCCNC3)nc12] |
| I-6 | 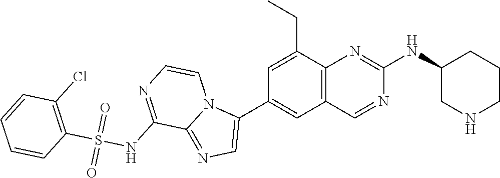 [SMILES: CCc1cc(-c2cnc3c(NS(=O)(=O)c4ccccc4Cl)nccn23)cc2cnc(N[C@H]3CCCNC3)nc12] |
| I-7 | 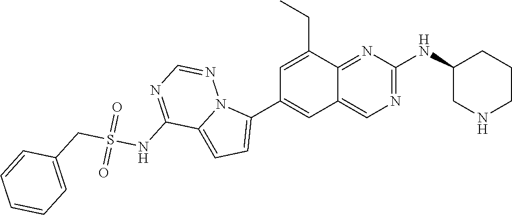 [SMILES: CCc1cc(-c2ccc3c(NS(=O)(=O)Cc4ccccc4)ncnn23)cc2cnc(N[C@H]3CCCNC3)nc12] |
| I-8 | 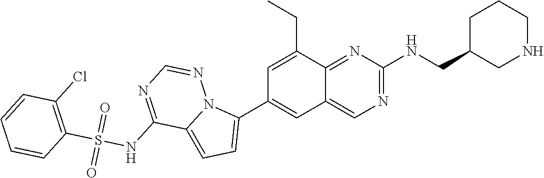 [SMILES: CCc1cc(-c2ccc3c(NS(=O)(=O)c4ccccc4Cl)ncnn23)cc2cnc(NC[C@H]3CCCNC3)nc12] |
| I-9 | 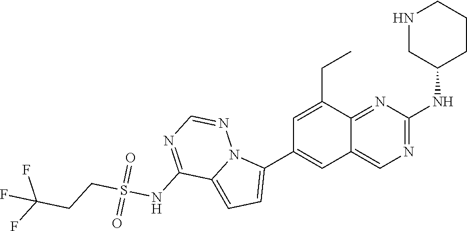 [SMILES: CCc1cc(-c2ccc3c(NS(=O)(=O)CCC(F)(F)F)ncnn23)cc2cnc(N[C@H]3CCCNC3)nc12] |
| I-10 | 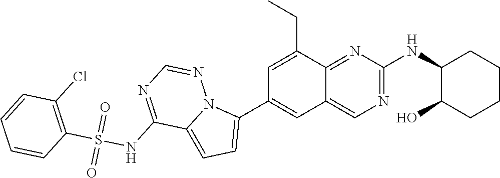 [SMILES: CCc1cc(-c2ccc3c(NS(=O)(=O)c4ccccc4Cl)ncnn23)cc2cnc(N[C@H]3CCCC[C@H]3O)nc12] |
| I-11 | 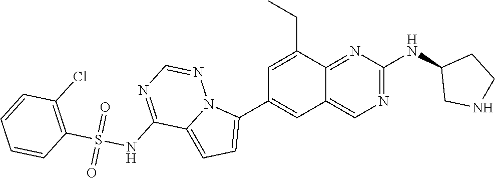 [SMILES: CCc1cc(-c2ccc3c(NS(=O)(=O)c4ccccc4Cl)ncnn23)cc2cnc(N[C@H]3CCNC3)nc12] |
| I-12 | 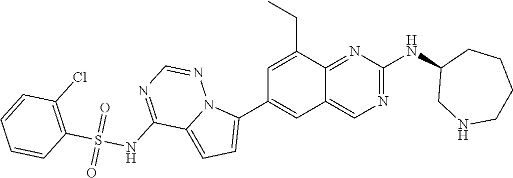 [SMILES: CCc1cc(-c2ccc3c(NS(=O)(=O)c4ccccc4Cl)ncnn23)cc2cnc(N[C@H]3CCCCNC3)nc12] |
| I-13 | 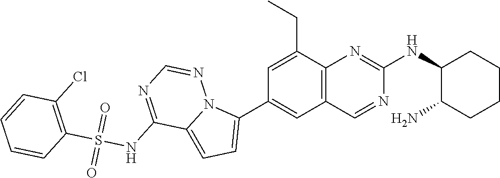 [SMILES: CCc1cc(-c2ccc3c(NS(=O)(=O)c4ccccc4Cl)ncnn23)cc2cnc(N[C@H]3CCCC[C@@H]3N)nc12] |
| I-14 | 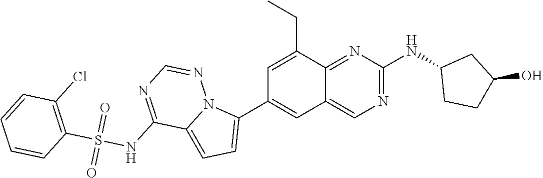 [SMILES: CCc1cc(-c2ccc3c(NS(=O)(=O)c4ccccc4Cl)ncnn23)cc2cnc(N[C@H]3CC[C@H](O)C3)nc12] |
| I-15 | 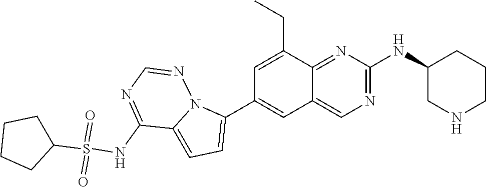 |
| I-16 | 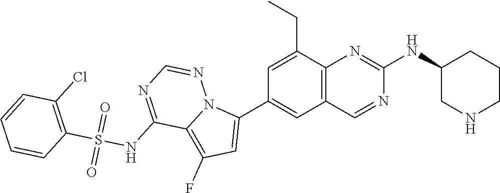 [SMILES: CCc1cc(-c2cc(F)c3c(NS(=O)(=O)c4ccccc4Cl)ncnn23)cc2cnc(N[C@H]3CCCNC3)nc12] |
| I-17 | 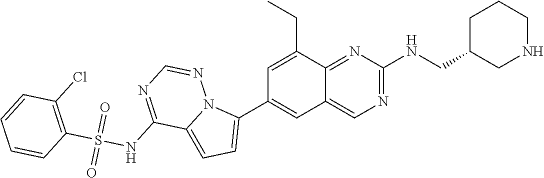 [SMILES: CCc1cc(-c2ccc3c(NS(=O)(=O)c4ccccc4Cl)ncnn23)cc2cnc(NC[C@@H]3CCCNC3)nc12] |
| I-18 | 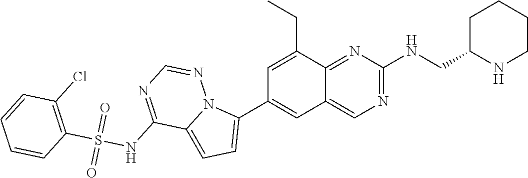 [SMILES: CCc1cc(-c2ccc3c(NS(=O)(=O)c4ccccc4Cl)ncnn23)cc2cnc(NC[C@@H]3CCCCN3)nc12] |
| I-19 | 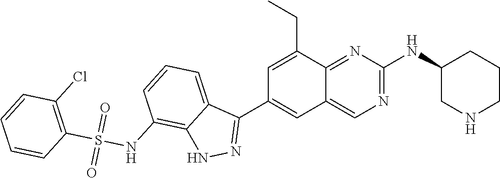 [SMILES: CCc1cc(-c2n[nH]c3c(NS(=O)(=O)c4ccccc4Cl)cccc23)cc2cnc(N[C@H]3CCCNC3)nc12] |
| I-20 | 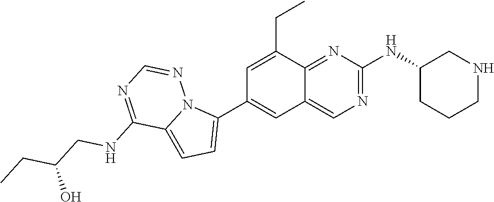 [SMILES: CCc1cc(-c2ccc3c(NC[C@H](O)CC)ncnn23)cc2cnc(N[C@H]3CCCNC3)nc12] |
| I-21 | 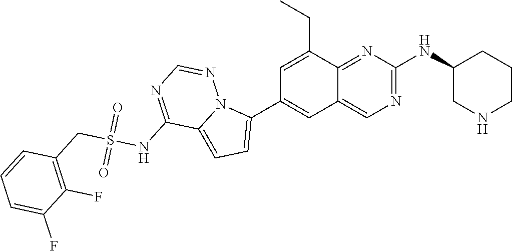 [SMILES: CCc1cc(-c2ccc3c(NS(=O)(=O)Cc4cccc(F)c4F)ncnn23)cc2cnc(N[C@H]3CCCNC3)nc12] |
| I-22 | 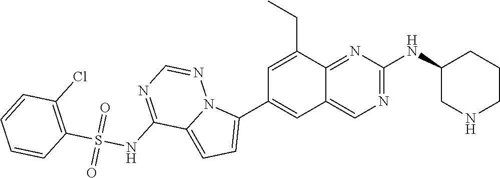 [SMILES: CCc1cc(-c2ccc3c(NS(=O)(=O)c4ccccc4Cl)ncnn23)cc2cnc(N[C@H]3CCCNC3)nc12] |
| I-23 | 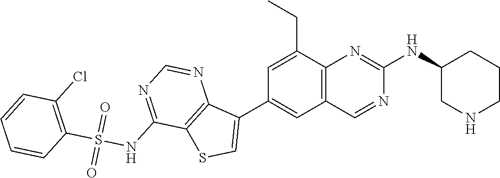 [SMILES: CCc1cc(-c2csc3c(NS(=O)(=O)c4ccccc4Cl)ncnc23)cc2cnc(N[C@H]3CCCNC3)nc12] |
| I-24 | 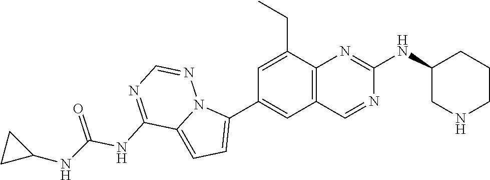 [SMILES: CCc1cc(-c2ccc3c(NC(=O)NC4CC4)ncnn23)cc2cnc(N[C@H]3CCCNC3)nc12] |
| I-25 | 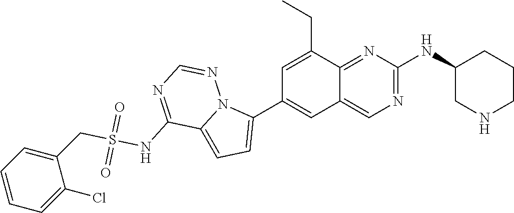 [SMILES: CCc1cc(-c2ccc3c(NS(=O)(=O)Cc4ccccc4Cl)ncnn23)cc2cnc(N[C@H]3CCCNC3)nc12] |
| I-26 | 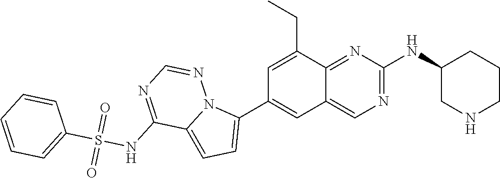 [SMILES: CCc1cc(-c2ccc3c(NS(=O)(=O)c4ccccc4)ncnn23)cc2cnc(N[C@H]3CCCNC3)nc12] |
| I-27 | 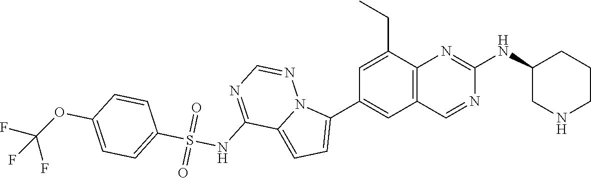 [SMILES: CCc1cc(-c2ccc3c(NS(=O)(=O)c4ccc(OC(F)(F)F)cc4)ncnn23)cc2cnc(N[C@H]3CCCNC3)nc12] |
| I-28 | 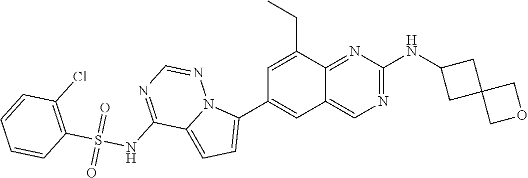 [SMILES: CCc1cc(-c2ccc3c(NS(=O)(=O)c4ccccc4Cl)ncnn23)cc2cnc(NC3CC4(COC4)C3)nc12] |
| I-29 | 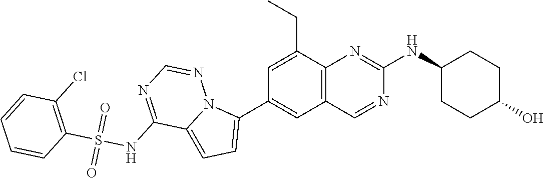 [SMILES: CCc1cc(-c2ccc3c(NS(=O)(=O)c4ccccc4Cl)ncnn23)cc2cnc(N[C@H]3CC[C@H](O)CC3)nc12] |
| I-30 | 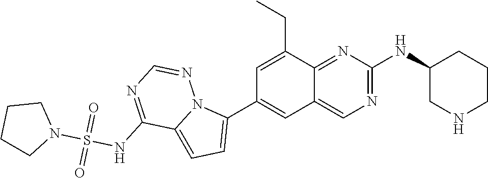 [SMILES: CCc1cc(-c2ccc3c(NS(=O)(=O)N4CCCC4)ncnn23)cc2cnc(N[C@H]3CCCNC3)nc12] |
| I-31 | 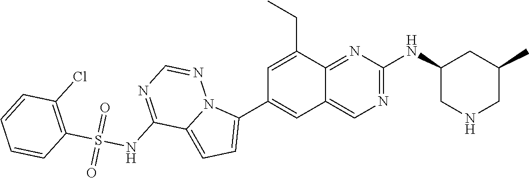 [SMILES: CCc1cc(-c2ccc3c(NS(=O)(=O)c4ccccc4Cl)ncnn23)cc2cnc(N[C@@H]3CNC[C@H](C)C3)nc12] |
| I-32 | 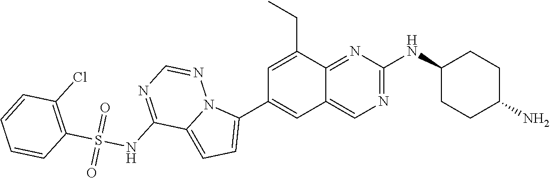 [SMILES: CCc1cc(-c2ccc3c(NS(=O)(=O)c4ccccc4Cl)ncnn23)cc2cnc(N[C@H]3CC[C@H](N)CC3)nc12] |
| I-33 | 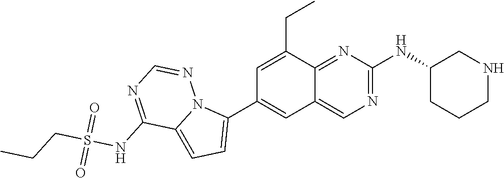 [SMILES: CCCS(=O)(=O)Nc1ncnn2c(-c3cc(CC)c4nc(N[C@H]5CCCNC5)ncc4c3)ccc12] |
| I-34 | 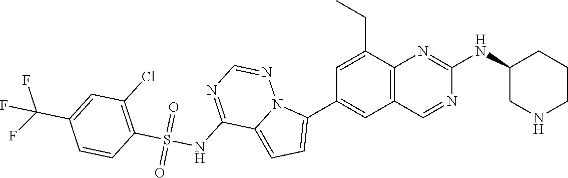 [SMILES: CCc1cc(-c2ccc3c(NS(=O)(=O)c4ccc(C(F)(F)F)cc4Cl)ncnn23)cc2cnc(N[C@H]3CCCNC3)nc12] |

or a pharmaceutically acceptable salt thereof.

20. A pharmaceutical composition comprising a compound according to claim 1, or a pharmaceutically acceptable salt thereof, and a pharmaceutically acceptable carrier, adjuvant, or vehicle.

21. (canceled)

22. A method of modulating IRE1α in a biological sample comprising contacting the sample with the compound of claim 1, or a pharmaceutically acceptable salt thereof.

23. A method of treating a IRE1α-mediated disorder, disease, or condition in a patient, comprising administering to said patient the compound of claim 1, or a pharmaceutically acceptable salt thereof.

24. The method of claim 23 wherein the disorder is a cell proliferation disorder, cancer, an autoimmune disorder, cardiovascular disease, viral infection, inflammation, a metabolism/endocrine disorder, a neurological disorder, neurodegenerative diseases, demyelinating disease, eye disease, fibrotic disease, diabetes, liver dysfunction, brain ischemia, or heart ischemia.

25. The method of claim 24 wherein the neurodegenerative disease is retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru.

26. The method of claim 24 wherein the demyelinating disease is Wolfram Syndrome, Pelizaeus-Merzbacher Disease, Transverse Myelitis, Charcot-Marie-Tooth Disease, or Multiple Sclerosis.

27. The method of claim 24 wherein the fibrotic disease is idiopathic pulmonary fibrosis (IPF), myocardial infarction, cardiac hypertrophy, heart failure, cirrhosis, acetominophen liver toxicity, hepatitis C liver disease, hepatosteatosis (fatty liver disease), or hepatic fibrosis.

28. The method of claim 24 wherein the cancer is selected from the group consisting of squamous cell cancer, small-cell lung cancer, non-small cell lung cancer (“NSCLC”), adenocarcinoma of the lung and squamous carcinoma of the lung, cancer of the peritoneum, hepatocellular cancer, stomach cancer, gastrointestinal cancer, esophageal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

29. The method of claim 24 wherein the cancer is multiple myeloma, ovarian cancer, breast cancer, or triple negative breast cancer (TNBC).
